(19) United States
(12) Patent Application Publication
(10) Pub. No.: US 2026/0344627 A1
(43) Pub. Date: Nov. 5, 2026

(54) TRANS-SPLICING METHODS AND COMPOSITIONS FOR GENERATION OF SINGLE SEX OFFSPRING

(71) Applicant: Joseph Fenton LAWLER, Lakeway, TX (US)

(72) Inventor: Joseph Fenton LAWLER, Lakeway, TX (US)

(21) Appl. No.: 19/662,256

(22) Filed: Apr. 29, 2026

Related U.S. Application Data
(60) Provisional application No. 63/798,280, filed on May 1, 2025.

(51) Int. Cl. A01K 67/0278; C07K 14/34; C07K 14/415; C12N 9/22; C12N 9/64; C12N 9/76; C12N 15/11; C12N 15/85; C12N 15/90

(52) U.S. Cl. CPC A01K 67/0278; C07K 14/34; C07K 14/415; C12N 9/224; C12N 9/226; C12N 9/641; C12N 9/6427; C12N 15/111; C12N 15/8509; C12N 15/907; A01K 2217/072; A01K 2227/30; C07K 2319/85; C12N 2310/20; C12N 2830/42; C12Y 304/21001; C12Y 304/21004; C12Y 304/22002

(57) ABSTRACT

Described herein are methods and compositions for generating single sex offspring using a trans-splicing approach, which can be enhanced by the use of one or more of a protein intermediary such as an RNA binding framework. Provided herein are methods and compositions to generate single sex offspring that are not genetically modified. These techniques can be applied to compassionate animal breeding.

### CROSS REFERENCE

This application claims priority to U.S. Provisional Application Ser. No. 63/798,280, filed May 1, 2025, which is incorporated herein by reference in its entirety.

### BACKGROUND

In many agricultural applications it is desirable to generate single sex offspring. For example, the products of a mating between two chicken lines that are optimized for certain egg laying characteristics are only useful when the offspring are female because males cannot lay eggs and breeding lines used for egg laying are generally not optimized for meat production. As a result, male chicks are separated from females as soon after hatching as possible and culled. Because male and female chicks do not look appreciably different for several days to weeks after hatching, specialized methods must be employed to distinguish them. One of the oldest methods involves the use of chicken sexers. These are skilled individuals who are able to determine the sex of a chick before it is obvious to an untrained person. Still, despite their acumen, chicken sexers are not perfect. Moreover, even if chicken sexers were perfectly accurate, they still must wait for the egg to hatch which means eggs harboring males must be carried through the entire incubation process before being identified and culled. This wastes resources on eggs that are not suitable for producing egg laying hens. Thus, finding new methods to generate single sex offspring of animals such as chickens is needed.

### SUMMARY OF THE DISCLOSURE

To meet the need to generate single sex offspring, disclosed herein, in certain aspects, are methods and compositions using trans-splicing process. In one aspect, the present disclosure provides a non-human vertebrate animal having a modified genotype comprising: one or more first sequence variants of a gene, wherein the one or more first sequence variants of the gene comprise a modified intron that is not capable of base pairing with a trans-splicing accepting gene; and one or more expression cassettes comprising: a nucleic acid sequence encoding a replicon RNA (repRNA), comprising a nucleic acid sequence encoding two or more transgenic proteins, two or more splice sites, and one or more introns, wherein the two or more transgenic proteins comprise a first transgenic protein and a second transgenic protein; wherein a 5′ end of the nucleic acid sequence encoding the first transgenic protein comprises a first splice site and a 3′ end of the nucleic acid sequence encoding the first transgenic protein comprises a second splice site; wherein at least one of the one or more introns does not comprise a stop codon; and wherein at least one of the one or more introns comprises a sequence that is complementary to one or more second sequence variants of the gene.

In some embodiments, translation of the first transgenic protein, but not the second transgenic protein, is initiated when RNA trans-splicing occurs at both the first splice site and the second splice site. In some embodiments, translation of each of the first transgenic protein and the second transgenic protein is initiated when RNA trans-splicing occurs only at the first splice site. In some embodiments, the first transgenic protein is located on a 5′ end of the second transgenic protein. In some embodiments, the first splice site is an acceptor splice site. In some embodiments, the second splice site is a donor splice site.

In some embodiments, the first transgenic protein is different than the second transgenic protein. In some embodiments, the first transgenic protein is a reporter gene. In some embodiments, the first transgenic protein is a toxin. In some embodiments, the toxin comprises a nuclease or a protease. In some embodiments, the nuclease comprises a Barnase or an RNase. In some embodiments, the protease comprises a caspase, trypsin, chymotrypsin, or papain. In some embodiments, the second transgenic protein is a cognate toxin inhibitor. In some embodiments, the first transgenic protein is a Barnase or a variant thereof, and the second transgenic protein is a Barstar or a variant thereof. In some embodiments, the first transgenic protein is an RNase, and the second transgenic protein is an RNase inhibitor. In some embodiments, (i) the first transgenic protein is a restriction endonuclease, and the second transgenic protein is a restriction endonuclease inhibitor; (ii) the first transgenic protein is a diphtheria, and the second transgenic protein is a diphtheria antitoxin; (iii) the first transgenic protein is a ricin, and the second transgenic protein is a ricin inhibitor; (iv) the first transgenic protein is an abrin, and the second transgenic protein is an abrin inhibitor; (v) the first transgenic protein is a pokeweed antiviral protein, and the second transgenic protein is a pokeweed antiviral protein inhibitor; (vi) the first transgenic protein is a caspase, and the second transgenic protein is a caspase inhibitor; (vii) the first transgenic protein is a trypsin, and the second transgenic protein is a trypsin inhibitor; (viii) the first transgenic protein is a chymotrypsin, and the second transgenic protein is a chymotrypsin inhibitor; or (ix) the first transgenic protein is a papain, and the second transgenic protein is a papain inhibitor.

In some embodiments, the one or more expression cassettes further comprises a second nucleic acid sequence encoding a Cas polypeptide linked to an RNA Binding Protein (RBP) and a third nucleic acid sequence encoding a guide RNA (gRNA) capable of directing one or more CRISPR RNA-guided complexes encoded by the one or more expression cassettes to one or more second sequence variants of the gene. In some embodiments, the one or more introns comprises a first intron located on the 5′ end of the first transgenic protein, and wherein the first intron comprises a first RBP-binding hairpin. In some embodiments, the one or more introns comprises a second intron located on the 3′ end of the first transgenic protein, and wherein the second intron comprises a second RBP-binding hairpin. In some embodiments, a second RBP-binding hairpin is on a nucleic acid sequence located on a 3′ end of the second transgenic protein. In some embodiments, a second RBP-binding hairpin is on a nucleic acid sequence located on the 5′ end of the second transgenic protein. In some embodiments, the one or more introns comprises a first intron located on the 3′ end of the first transgenic protein, and wherein the first intron comprises a first RBP-binding hairpin.

In some embodiments, the one or more expression cassettes comprise a promoter that is expressed during embryogenesis. In some embodiments, the one or more expression cassettes comprises an inducible promoter.

In some embodiments, the gene is a non-essential gene. In some embodiments, the gene is an essential gene. In some embodiments, the gene is expressed in an embryo. In some embodiments, the gene is a housekeeping gene that is constitutively expressed.

In some embodiments, the non-human vertebrate animal is selected from the group consisting of cow, mouse, rat, rabbit, guinea pig, chicken, fish, bird, reptile, camelid, bovine, chimpanzee, sheep, goat, and non-human primate.

In some embodiments, the one or more second sequence variants of the gene do not have sequence identity to the one or more first sequence variant of the gene. In some embodiments, the one or more second sequence variants of the gene have sequence identity to a wildtype sequence of the gene.

In some embodiments, the RNA trans-splicing process is spliceosome mediated. In some embodiments, the RNA trans-splicing process is ribozyme mediated. In some embodiments, the Cas polypeptide is a Cas endonuclease. In some embodiments, the Cas endonuclease is a class II Cas endonuclease. In some embodiments, the Cas endonuclease is a type II, type III, or type VI Cas endonuclease. In some embodiments, the Cas endonuclease is an RNA-guided RNA endonuclease. In some embodiments, the Cas endonuclease is selected from the group consisting of Cas9, Cas13, Csm/Cmr, Cas7-11, DisCas7-11, and Cas12a. In some embodiments, the Cas7-11 is Cas7-11a, Cas7-11b, Cas7-11c, or Cas7-11d. In some embodiments, the Cas polypeptide is a variant of the Cas endonuclease. In some embodiments, the Cas polypeptide is an inactive form of the Cas endonuclease. In some embodiments, the Cas polypeptide binds to a polynucleotide but does not cleave the polynucleotide. In some embodiments, the Cas polypeptide is a deactivated Cas13 (dCas13) or a deactivated dDisCas7-11 (dDisCas7-11). In some embodiments, the Cas polypeptide is a dCas13a, dCas13b, dCas13c, or dCas13d. In some embodiments, the Cas polypeptide is a variant of a Prevotella sp. Cas13b (PspCas13b).

In some embodiments, the RBP is selected from the group consisting of MS2 coat protein (MCP), PP7 bacteriophage coat protein, small RNA phage PRR1, and RNA bacteriophages QB coat protein. In some embodiments, the number of the RBP-binding hairpins is at least 1, at least 2, at least 3, at least 4, at least 5, at least 6, at least 7, at least 8, at least 9, or at least 10. In some embodiments, the one or more CRISPR RNA-guided complexes comprise the guide RNA, the Cas polypeptide, the repRNA, or a combination thereof.

In some embodiments, the gene is an autosomal gene. In some embodiments, the gene is an allosomal gene. In some embodiments, the one or more expression cassettes is located on an autosomal chromosome. In some embodiments, the one or more expression cassettes is located on an allosomal chromosome.

In another aspect, provided herein is a plurality of non-human vertebrate animals comprising: a first non-human vertebrate animal having a genotype comprising: one or more first sequence variants of a gene, wherein the one or more first sequence variants of the gene comprise a modified intron that is not capable of base pairing with a trans-splicing accepting gene; and (ii) one or more expression cassettes comprising: a nucleic acid sequence encoding a replicon RNA (repRNA), comprising a nucleic acid sequence encoding two or more transgenic proteins, two or more splice sites, and one or more introns, wherein the two or more transgenic proteins comprise a first transgenic protein and a second transgenic protein; wherein a 5′ end of the nucleic acid sequence encoding the first transgenic protein comprises a first splice site and a 3′ end of the nucleic acid sequence encoding the first transgenic protein comprises a second splice site; wherein at least one of the one or more introns does not comprise a stop codon; and wherein at least one of the one or more introns comprises a sequence that is complementary to one or more second sequence variants of the gene; and (b) a second non-human vertebrate animal comprising the one or more second variants of the gene.

In some embodiments, translation of the first transgenic protein, but not the second transgenic protein, is initiated when RNA trans-splicing occurs at both the first splice site and the second splice site. In some embodiments, translation of each of the first transgenic protein and the second transgenic protein is initiated when RNA trans-splicing occurs only at the first splice site. In some embodiments, the first transgenic protein is located on a 5′ end of the second transgenic protein. In some embodiments, the first splice site is an acceptor splice site. In some embodiments, the second splice site is a donor splice site.

In some embodiments, the first transgenic protein is different than the second transgenic protein. In some embodiments, the first transgenic protein is a reporter gene. In some embodiments, the first transgenic protein is a toxin. In some embodiments, the toxin comprises a nuclease or a protease. In some embodiments, the nuclease comprises a Barnase or an RNase. In some embodiments, the protease comprises a caspase, trypsin, chymotrypsin, or papain. In some embodiments, the second transgenic protein is a cognate toxin inhibitor. In some embodiments, the first transgenic protein is a Barnase or a variant thereof, and the second transgenic protein is a Barstar or a variant thereof. In some embodiments, the first transgenic protein is an RNase, and the second transgenic protein is an RNase inhibitor. In some embodiments, (i) the first transgenic protein is a restriction endonuclease, and the second transgenic protein is a restriction endonuclease inhibitor; (ii) the first transgenic protein is a diphtheria, and the second transgenic protein is a diphtheria antitoxin; (iii) the first transgenic protein is a ricin, and the second transgenic protein is a ricin inhibitor; (iv) the first transgenic protein is an abrin, and the second transgenic protein is an abrin inhibitor; (v) the first transgenic protein is a pokeweed antiviral protein, and the second transgenic protein is a pokeweed antiviral protein inhibitor; (vi) the first transgenic protein is a caspase, and the second transgenic protein is a caspase inhibitor; (vii) the first transgenic protein is a trypsin, and the second transgenic protein is a trypsin inhibitor; (viii) the first transgenic protein is a chymotrypsin, and the second transgenic protein is a chymotrypsin inhibitor; or (ix) the first transgenic protein is a papain, and the second transgenic protein is a papain inhibitor.

In some embodiments, the one or more expression cassettes further comprises a second nucleic acid sequence encoding a Cas polypeptide linked to an RNA Binding Protein (RBP) and a third nucleic acid sequence encoding a guide RNA (gRNA) capable of directing one or more CRISPR RNA-guided complexes encoded by the one or more expression cassettes to one or more second sequence variants of the gene. In some embodiments, the one or more introns comprises a first intron located on the 5′ end of the first transgenic protein, and wherein the first intron comprises a first RBP-binding hairpin. In some embodiments, the one or more introns comprises a second intron located on the 3′ end of the first transgenic protein, and wherein the second intron comprises a second RBP-binding hairpin. In some embodiments, a second RBP-binding hairpin is on a nucleic acid sequence located on a 3′ end of the second transgenic protein. In some embodiments, a second RBP-binding hairpin is on a nucleic acid sequence located on the 5′ end of the second transgenic protein. In some embodiments, the one or more introns comprises a first intron located on the 3′ end of the first transgenic protein, and wherein the first intron comprises a first RBP-binding hairpin.

In some embodiments, the one or more expression cassettes comprise a promoter that is expressed during embryogenesis. In some embodiments, the one or more expression cassettes comprises an inducible promoter.

In some embodiments, the gene is a non-essential gene. In some embodiments, the gene is an essential gene. In some embodiments, the gene is expressed in an embryo. In some embodiments, the gene is a housekeeping gene that is constitutively expressed.

In some embodiments, the non-human vertebrate animal is selected from the group consisting of cow, mouse, rat, rabbit, guinea pig, chicken, fish, bird, reptile, camelid, bovine, chimpanzee, sheep, goat, and non-human primate.

In some embodiments, the one or more second sequence variants of the gene do not have sequence identity to the one or more first sequence variant of the gene. In some embodiments, the one or more second sequence variants of the gene have sequence identity to a wildtype sequence of the gene. In some embodiments, the RNA trans-splicing process is spliceosome mediated. In some embodiments, the RNA trans-splicing process is ribozyme mediated.

In some embodiments, the Cas polypeptide is a Cas endonuclease. In some embodiments, the Cas endonuclease is a class II Cas endonuclease. In some embodiments, the Cas endonuclease is a type II, type III, or type VI Cas endonuclease. In some embodiments, the Cas endonuclease is an RNA-guided RNA endonuclease. In some embodiments, the Cas endonuclease is selected from the group consisting of Cas9, Cas13, Csm/Cmr, Cas7-11, DisCas7-11, and Cas12a. In some embodiments, the Cas7-11 is Cas7-11a, Cas7-11b, Cas7-11c, or Cas7-11d. In some embodiments, the Cas polypeptide is a variant of the Cas endonuclease. In some embodiments, the Cas polypeptide is an inactive form of the Cas endonuclease. In some embodiments, the Cas polypeptide binds to a polynucleotide but does not cleave the polynucleotide. In some embodiments, the Cas polypeptide is a deactivated Cas13 (dCas13) or a deactivated dDisCas7-11 (dDisCas7-11). In some embodiments, the Cas polypeptide is a dCas13a, dCas13b, dCas13c, or dCas13d. In some embodiments, the Cas polypeptide is a variant of a Prevotella sp. Cas13b (PspCas13b).

In some embodiments, the RBP is selected from the group consisting of MS2 coat protein (MCP), PP7 bacteriophage coat protein, small RNA phage PRR1, and RNA bacteriophages QB coat protein. In some embodiments, the number of the RBP-binding hairpins is at least 1, at least 2, at least 3, at least 4, at least 5, at least 6, at least 7, at least 8, at least 9, or at least 10. In some embodiments, the one or more CRISPR RNA-guided complexes comprise the guide RNA, the Cas polypeptide, the repRNA, or a combination thereof.

In some embodiments, the gene is an autosomal gene. In some embodiments, the gene is an allosomal gene. In some embodiments, the one or more expression cassettes is located on an autosomal chromosome. In some embodiments, the one or more expression cassettes is located on an allosomal chromosome.

In another aspect, provided herein is a method of producing a single sex population of non-human vertebrate animals, the method comprising: crossing the first non-human vertebrate animal described herein; with the second non-human vertebrate animal described herein; wherein a resulting progeny having a genotype comprising the one or more second sequence variants of the gene and the one or more expression cassettes is not viable; thereby producing the single sex population of non-human vertebrate animals. In some embodiments, each of the non-human vertebrate animals of the single sex population of non-human vertebrate animals is not genetically engineered.

In various aspect, provided herein is a non-human vertebrate animal having a modified genotype comprising: one or more first sequence variants of a gene, wherein the one or more first sequence variants of the gene comprise a modified intron that is not capable of base pairing with a trans-splicing accepting gene; and one or more expression cassettes comprising: a nucleic acid sequence encoding a replicon RNA (repRNA), comprising a nucleic acid sequence encoding two or more transgenic proteins, two or more splice sites, and one or more introns, wherein the two or more transgenic proteins comprise a first transgenic protein and a second transgenic protein; wherein a 5′ end of the nucleic acid sequence encoding the first transgenic protein comprises a first splice site and a 3′ end of the nucleic acid sequence encoding the first transgenic protein comprises a second splice site; wherein the first transgenic protein is located on a 5′ end of the second transgenic protein; wherein at least one of the one or more introns does not comprise a stop codon;

and wherein at least one of the one or more introns comprises a sequence that is complementary to one or more second sequence variants of the gene.

In one aspect, provided herein is a non-human vertebrate animal having a modified genotype comprising: one or more first sequence variants of a gene, wherein the one or more first sequence variants of the gene comprise a modified intron that is not capable of base pairing with a trans-splicing accepting gene; and one or more expression cassettes comprising: a first nucleic acid sequence encoding a Cas polypeptide linked to an RNA Binding Protein (RBP); (ii) a second nucleic acid sequence encoding a replicon RNA (repRNA), comprising a nucleic acid sequence encoding two or more transgenic proteins, two or more splice sites, and one or more introns, wherein the two or more transgenic proteins comprise a first transgenic protein and a second transgenic protein; wherein a 5′ end of the nucleic acid sequence encoding the first transgenic protein comprises a first splice site and a 3′ end of the nucleic acid sequence encoding the first transgenic protein comprises a second splice site; and wherein at least one of the one or more introns (a) is connected to an RBP-binding hairpin, and/or (b) does not comprise a stop codon; and (iii) a third nucleic acid sequence encoding a guide RNA (gRNA) capable of directing one or more CRISPR RNA-guided complexes encoded by the one or more expression cassettes to one or more second sequence variants of the gene.

In another aspect, provided herein is a plurality of non-human vertebrate animals comprising: a first non-human vertebrate animal having a genotype comprising one or more first sequence variants of a gene, wherein the one or more first sequence variants of the gene comprise a modified intron that is not capable of base pairing with a trans-splicing accepting gene; and (ii) one or more expression cassettes comprising: a first nucleic acid sequence encoding a Cas polypeptide linked to an RNA Binding Protein (RBP); a second nucleic acid sequence encoding a replicon RNA (repRNA), comprising a nucleic acid sequence encoding two or more transgenic proteins, two or more splice sites, and one or more introns, wherein the two or more transgenic proteins comprise a first transgenic protein and a second transgenic protein; wherein a 5′ end of the nucleic acid sequence encoding the first transgenic protein comprise a first splice site and a 3′ end of the nucleic acid sequence encoding the first transgenic protein comprises a second splice site; and wherein at least one of the one or more introns (a) is connected to an RBP-binding hairpin, and/or (b) does not comprise a stop codon; and a third nucleic acid sequence encoding a guide RNA (gRNA) capable of directing one or more CRISPR RNA-guided complexes encoded by the one or more expression cassettes to one or more second sequence variants of the gene; and a second non-human vertebrate animal comprising the one or more second variants of the gene.

In various aspects, provided herein is a non-human vertebrate animal having a modified genotype comprising: one or more first sequence variants of a gene, wherein the one or more first sequence variants of the gene comprise a modified intron that is not capable of base pairing with a trans-splicing accepting gene; and one or more expression cassettes comprising: a first nucleic acid sequence encoding a Cas polypeptide linked to an RNA Binding Protein (RBP); (ii) a second encoding two or more transgenic proteins, two or more splice sites, and one or more introns, wherein the two or more transgenic proteins comprise a first transgenic protein and a second transgenic protein; wherein a 5′ end of the nucleic acid sequence encoding the first transgenic protein comprises a first splice site and a 3′ end of the nucleic acid sequence encoding the first transgenic protein comprises a second splice site; and wherein at least one of the one or more introns (a) is connected to an RBP-binding hairpin, and/or (b) does not comprise a stop codon; wherein at least one of the one or more introns comprises a sequence that is complementary to one or more second sequence variants of the gene; and (iii) a third nucleic acid sequence encoding a guide RNA (gRNA) capable of directing one or more CRISPR RNA-guided complexes encoded by the one or more expression cassettes to one or more third sequence variants of the gene, wherein the one or more second sequence variants of the gene and the one or more third sequence variants of the gene do not have sequence identity to each other.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

An understanding of the features and advantages of the present invention will be obtained by reference to the following detailed description that sets forth illustrative embodiments, in which the principles of the invention are utilized, and the accompanying drawings of which:

FIG. 1 shows a diagram of a cross between a genetically modified female animal with allosomes W (the modification is located on the chromosome) and a wildtype male animal with allosomes ZZ. The resulting progeny with allosomes Z are not viable whereas the resulting progeny with allosomes ZW are viable. Thus, a generation of single sex offspring is achieved.

FIG. 2. shows an overview of an enhanced trans-splicing scheme via an RNA binding framework. In this example, CRISPR-mediated trans-splicing is achieved by binding the CRISPR-Cas ribonucleoproteins (RNP) and replicon RNA (repRNA) complex to a target pre-mRNA.

FIGS. 3A-3E show an overview of a trans-splicing construct designed to differentiate between on-target and off-target trans-splicing events. FIG. 3A shows an exemplary diagram of a trans-splicing construct carrying two transgenes. Trans-splicing sites are located on each of the 5′ end of the first transgene (not shown) and on the 3′ end of the first transgene (donor splice site or “dss”). FIG. 3B shows an overview of a pairing of the trans-splicing construct to the targeted pre-mRNA. FIG. 3C shows a diagram of a trans-splicing event when the trans-splicing occurs only at intron n on the first trans-splice site (acceptor splice site or “ass,” on the 5′ end of the first transgene). FIG. 3D shows a diagram of a trans-splicing event when the trans-splicing occurs only at intron n+1 on the dss (on the 3′ end of the first transgene). FIG. 3E shows a diagram of a trans-splicing event when both 5′ trans-splicing (at the ass) and 3′ trans-splicing (at the dss) occur.

FIGS. 4A-4C show exemplary diagrams of a trans-splicing construct with an RNA binding framework designed to improve trans-splicing efficiency and accuracy. FIG. 4A shows a diagram of a trans-splicing construct with a first RBP-binding hairpin located on the 5′ end of the first transgene and a second RBP-binding hairpin located on the 3′ end of the second transgene. FIG. 4B shows a diagram of a trans-splicing construct with a first RBP-binding hairpin located on the 5′ end of the first transgene and a second RBP-binding hairpin located on the 3′ end of the first transgene. FIG. 4C shows a diagram of a trans-splicing construct with an RBP-binding hairpin located on the 5′ end of the first transgene. A sequence that is complementary to an intron of the target pre-mRNA is located on the 3′ end of the first transgene.

### DETAILED DESCRIPTION OF THE DISCLOSURE

In many agricultural applications, generation of single sex offspring, for example, female hens, is desirable. The products of a mating between two chicken lines optimized for certain egg laying characteristics are useful when the offspring are female because males cannot lay eggs and are generally not optimized for meat production. As a result, male chicks are separated from female chicks as soon after hatching as possible and culled.

According to the statistical report from United States Department of Agriculture (USDA), U.S. egg production totaled 8.67 billion eggs during June 2022, and the total layer hens in the U.S. on Jul. 1, 2022, is about 366 million (USDA. Chickens and Eggs. July 2022. ISSN: 1948-9064). Layer hens, however, can only lay eggs from 18-19 weeks to 72-78 weeks of age. The layer hen industry, thus, requires the replacement of layer hens annually. Each year, approximately 221.6-million-layer hens must be replaced. Assuming an equal sex ratio, this means 523-million-layer hen eggs must be hatched, of which approximately half will be male. All male chicks will be culled. Each year, up to 300 million male chicks are culled in the U.S., and as many as 7 billion male chicks are culled globally. This presents both animal welfare and ethical issues. In fact, Germany has recently banned the culling of day-old male chicks and Italy plans to follow suit. Companies have also taken notice and recently announced they oppose the practice.

Because male and female chicks do not look appreciably different for at least several days to weeks after hatching, specialized methods must be employed to distinguish them. One of the oldest methods for doing so involves the use of chicken sexers. These are skilled individuals who are able to determine the sex of a chick before it is obvious to an untrained person. Still, despite their acumen, chicken sexers are not perfect, although some can reach greater than 90% accuracy.

Moreover, even if chicken sexers were perfectly accurate, they still must wait for the egg to hatch and grow for several days, which means eggs harboring males must be carried through the entire incubation process before being identified and culled. This wastes resources on eggs which eventually must be culled because male chicks are not suitable for the intended purpose.

Other methods have been developed to separate male and female chicks that rely on feather color (See Gohler, D. et al. 2017. Poult Sci. 1;96 (1): 1-4). Some methods involve the expression of marker proteins such as green fluorescent protein. Although selectively expressing green fluorescent protein will allow distinguishing between sexes, it will also result in genetically modified birds that may not be suitable to customers and are subject to greater regulation (See Kang, K. et al. “Production of chickens with green fluorescent protein-knockin in the Z chromosome and detection of green fluorescent protein-positive chicks in the embryonic stage.” Animal bioscience vol. 36,6 (2023): 973-979. Doi: 10.5713/ab.22.0405; see also Lee, H. et al. 2019. FASEB J. 33 (7): 8519-8529).

Methods to allow for selection at the egg stage have also been developed. These methods include detecting minute amounts of estrogen, DNA sequences and other analytes that identify the bird's sex (see e.g., M-E Krautwald-Junghanns, M. et al. 2018. Poult Sci.1;97 (3): 749-757). These methods, while effective, often require additional machinery for implementation and do not escape the fact that male eggs must be incubated to some point.

The present disclosure provides methods and compositions for an approach wherein a genetically modified female chicken (parent generation) can be mated with a male from any other chicken line and produce only female offspring. The female offspring from this cross are not genetically modified thereby avoiding potential consumer rejection over concerns about consuming genetically modified food. In one aspect, provided herein is a genetically modified female chicken line that can be used to breed with males from the same or different lines such that only female offspring is viable. In some instances, the methods and compositions described herein to generate a single sex population of animal, e.g., a chicken, can improve efficiency of layer hen production while reducing time, cost, and resources used. In another instance, the methods and compositions described herein provide an alternative approach to the culling of male chickens, which results in the death of billions of male chicks annually.

In one aspect, the present disclosure provides methods and compositions whereby eggs that would otherwise bear male chickens are suppressed by utilizing a trans-splicing process to express a transgene or gene of interest, in which such expressed is lethal to the cell. The methods and compositions described herein can be applied, modified, and utilized in other animals, including, but not limited to, cows, mice, rats, rabbits, guinea pigs, chicken, fish, birds, reptiles, camelids, bovine, chimpanzees, sheep, goats, and non-human primates.

In one aspect, the present disclosure provides methods and compositions wherein eggs that would otherwise bear male chickens fail to develop by utilizing a trans-splicing process. This approach would improve efficiency and require less resources to generate female hens. Eggs that bear male chicks are suppressed during embryogenesis thereby saving costs and resources required for egg hatching.

In another aspect, provided herein are one or more expression cassettes comprising trans-splicing constructs for generating a single sex population. In various aspects, provided herein are methods to generate a single sex population using one or more expression cassettes comprising trans-splicing constructs described herein. Because off-target trans-splicing events occur, in another aspect, provided herein is a trans-splicing construct that can be utilized to differentiate on-target trans-splicing events and off-target trans-splicing events. In some instances, the one or more expression cassettes comprise a nucleic acid sequence encoding two or more transgenic proteins comprising a first transgenic protein and a second transgenic protein. In another aspect, provided herein is a trans-splicing construct that can improve accuracy and efficiency of trans-splicing events.

In one aspect provided herein, is a non-human vertebrate animal having a modified genotype comprising: (1) one or more first sequence variants of a gene, wherein the one or more first sequence variants of the gene comprise a modified intron that is not capable of base pairing with a trans-splicing accepting gene; and (2) one or more expression cassettes comprising: a nucleic acid sequence encoding a replicon RNA (repRNA), comprising a nucleic acid sequence encoding two or more transgenic proteins, two or more splice sites, and one or more introns, wherein the two or more transgenic proteins comprise a first transgenic protein and a second transgenic protein; wherein a 5′ end of the nucleic acid sequence encoding the first transgenic protein comprises a first splice site and a 3′ end of the nucleic acid sequence encoding the first transgenic protein comprises a second splice site; wherein at least one of the one or more introns does not comprise a stop codon; and wherein at least one of the one or more introns comprises a sequence that is complementary to one or more second sequence variants of the gene. In another aspect, provided herein is a plurality of non-human vertebrate animals comprising: (a) a first non-human vertebrate animal having a genotype comprising: (i) one or more first sequence variants of a gene, wherein the one or more first sequence variants of the gene comprise a modified intron that is not capable of base pairing with a trans-splicing accepting gene; and (ii) one or more expression cassettes comprising: a nucleic acid sequence encoding a replicon RNA (repRNA), comprising a nucleic acid sequence encoding two or more transgenic proteins, two or more splice sites, and one or more introns, wherein the two or more transgenic proteins comprise a first transgenic protein and a second transgenic protein; wherein a 5′ end of the nucleic acid sequence encoding the first transgenic protein comprises a first splice site and a 3′ end of the nucleic acid sequence encoding the first transgenic protein comprises a second splice site; wherein at least one of the one or more introns does not comprise a stop codon; and wherein at least one of the one or more introns comprises a sequence that is complementary to one or more second sequence variants of the gene; and (b) a second non-human vertebrate animal comprising the one or more second variants of the gene.

In some instances, the one or more expression cassettes further comprises a second nucleic acid sequence encoding a Cas polypeptide linked to an RNA Binding Protein (RBP) and a third nucleic acid sequence encoding a guide RNA (gRNA) capable of directing one or more CRISPR RNA-guided complexes encoded by the one or more expression cassettes to one or more second sequence variants of the gene.

In various aspects, provided herein is a method of producing a single sex population of non-human vertebrate animals, the method comprising: crossing the first non-human vertebrate animal described herein with the second non-human vertebrate animal described herein, wherein a resulting progeny having a genotype comprising the one or more second sequence variants of the gene and the one or more expression cassettes is not viable, thereby producing the single sex population of non-human vertebrate animals. In various aspects, each of the non-human vertebrate animals of the single sex population of non-human vertebrate animals is not genetically engineered.

### DEFINITIONS

Unless defined otherwise, all technical and scientific terms used herein have the same meaning as commonly understood by one of ordinary skill in the art to which the invention pertains. Although any methods and materials similar or equivalent to those described herein can be used in the practice for testing of the present invention, the preferred materials and methods are described herein. In describing and claiming the present invention, the following terminology will be used.

It is also to be understood that the terminology used herein is for the purpose of describing particular embodiments only and is not intended to be limiting.

The articles “a” and “an” are used herein to refer to one or to more than one (i.e., to at least one) of the grammatical object of the article. By way of example, “an element” means one element or more than one element.

The term “about” and its grammatical equivalents in relation to a reference numerical value and its grammatical equivalents as used herein can include a range of values plus or minus 10% from that value. For example, the amount “about 10” includes amounts from 9 to 11. The term “about” in relation to a reference numerical value can also include a range of values plus or minus 10%, 9%, 8%, 7%, 6%, 5%, 4%, 3%, 2%, or 1% from that value.

As used herein, the terms “allosomes” or “allosome” refer to chromosome that determine sex of an offspring. Allosomes are sometimes referred to as sex chromosomes.

### Animal Genetics and Genotypes

The two categories of chromosomes are autosomes and allosomes (sex chromosomes). Autosomes are other chromosomes that are not allosomes. The allosomes carry the genetic material that determines the sex of an offspring. In mammals, such as humans, cows, or bovines, males are the heterogametic sex which means they have two different sex chromosomes X and Y. The mammalian Y chromosome is a crucial factor for determining sex in mammals. In this case, the female is determined by XX and the male is XY. However, in poultry species and reptiles, such as chickens, females are the heterogametic sex. The allosomes are referred to as Z and W. The female W chromosome in this case is instead an important factor for sex determination. The female chicken has the allosomes ZW while the male chicken has the allosomes ZZ. In male offspring, one of the Z chromosomes is derived from the male parent, while the other Z chromosome is derived from the female parent.

As used herein, strikethrough indication on the chromosomes, e.g., an allosome (or in poultry and reptile; or in mammals), refers to the chromosome, e.g., an allosome, that is integrated with one or more expression cassettes, e.g., an expression cassette carrying RNA trans-splicing construct. Additionally, in some instances, strikethrough allosome (or in poultry and reptile; or in mammals) comprises modification of a target gene so that RNA trans-splicing process cannot occur with the transcribed pre-mRNA.

As used herein, ¹ indication on the chromosomes, e.g., an allosome (Z¹ or W¹ in poultry and reptile; X¹ or Y¹ in mammals), refers to the chromosome, e.g., an allosome, that is integrated with one or more expression cassettes, e.g., an RNA trans-splicing expression cassette.

In some instances, as used herein, * indication on the chromosomes, e.g., an autosome such as A*, refers to the chromosome, e.g., an autosome, that has a mutated sequence that is not capable of base pairing with a trans-splicing accepting gene. In another instances, as used herein, * indication on the chromosomes, e.g., an autosome such as A*, refers to the chromosome, e.g., an autosome or an allosome, that has the mutated sequence so that the RNA trans-splicing cannot occur. In some instances, the mutated sequence is located in intronic regions. In some instances, the mutated sequence is not located in exons.

Chromosomes and genes come in pairs, and each parent contributes one gene in each pair of genes. If two copies of the genes are the same, the genotype or genetic state is referred to as homozygous. However, if two copies of the genes are different, the genotype in this case is referred to as heterozygous.

### Trans-Splicing Process

Trans-splicing is a special molecular process of RNA or protein where exons (in mRNA) or exteins (in protein) from two different primary mRNA transcripts (pre-mRNA) or proteins are cleaved to remove introns (in mRNA) or inteins (in protein) and joined end to end via ligation, resulting in a fusion mRNA or protein. Trans-splicing is less common than cis-splicing, which is a process in which the intronic removal occurs within the same primary mRNA transcript or protein molecule. Examples of applications utilizing trans-splicing include, but are not limited to, gene therapy for genetic diseases or generation of a single sex population of animals.

### RNA Trans-Splicing Process to Generate Single Sex Offspring

According to the central dogma of biology, DNA is transcribed into RNA, which is then translated into protein. However, many details are omitted from this statement because cellular messenger RNA species rarely align perfectly to the DNA from which they are transcribed. This is because the messenger RNA species in eukaryotic cells frequently undergo a post-transcriptional process called splicing.

RNA splicing is a biological process involving alteration of a precursor messenger RNA (pre-mRNA) transcript into a mature messenger RNA (mRNA). Pre-mRNA comprises introns and exons. During RNA splicing, the introns, or intervening sequences, which are non-coding regions of the mRNA, are spliced out, allowing the exons, which are protein coding regions, to join to become mature mRNA. In some cases, it is the mature mRNA that is translated into protein. In some cases, an intron is retained, and the non-spliced mRNA is translated into protein.

In some instances, the RNA splicing process occurs in cellular machinery called the spliceosome and is facilitated by small nuclear ribonucleoproteins (snRNPs). In some instances, the RNA splicing process occurs via ribozyme mediated process. RNA splicing process involves several steps. Briefly, introns are removed from pre-mRNA transcripts by cleavage at conserved sequences called splice sites. These splice sites are found at the 5′ and 3′ ends of introns. In some instances, the RNA sequence that is removed begins with the dinucleotide GU at its 5′ end and ends with AG at its 3′ end. In some instances, alternate splice site sequences are found that begin with the dinucleotide AU and end with AC. In some instances, at the 3′ splice site (or at the 5′ splice site relative to a gene of interest; or acceptor splice site), there are three consensus motif comprises: the branch point, polypyrimidine tract, and 3′ splice site. The branch point (BP), which is a sequence located anywhere from 18 to 40 nucleotides upstream from the 3′ end of an intron, also plays role in RNA splicing process. In some instances, the branch point comprises an adenine. In another instances, the BP sequence comprises YNYYRAY, where Y indicates a pyrimidine, N denotes any nucleotide, R denotes any purine, and A denotes adenine. The polypyrimidine tract is a region that promotes spliceosome assembly. Detailed RNA splicing process is described, for example, in Clancy, S. (2008). Nature Education 1 (1): 31; Yang, Y. et al. (2005). Molecular Therapy. 12 (6); Long, M. et al. (2003). J. Clin. Invest.; and Wally, V. et al. (2012). Journal of investigative Dermatology, each of which are hereby incorporated by reference of their entities.

There are broad categories of RNA splicing: RNA cis-splicing and RNA trans-splicing. In some instances, both RNA cis-splicing, and RNA trans-splicing processes share a similar mechanism. In RNA trans-splicing, two separate pre-mRNA, or in some instances, one pre-mRNA and one pre-trans-splicing molecule (PTM) carrying a transgene, are spliced, and joined, resulting in a fusion mature mRNA, which can express a protein encoded by the transgene.

In one aspect, the present disclosure provides methods and compositions for the generation of single sex offspring in animals by utilizing RNA trans-splicing process to express two or more transgenes or genes of interest, e.g., a toxin, in which when expressed is lethal to the cell. In some embodiments, translation of a first transgenic protein of the two or more transgenic proteins is initiated when RNA trans-splicing occurs at both the first splice site (e.g., a 5′ splice site relative to a gene of interest; acceptor splice site) and the second splice site (e.g., a 3′ splice site relative to a gene of interest; donor splice site). In another aspect, the present disclosure provides methods and compositions to generate a system utilizing RNA trans-splicing process whereby a line of chickens is genetically modified such that female chicken from this line can be mated with a male from any other chicken line and produce female offspring.

### Enhanced Trans-Splicing Process Via an RNA Binding Framework to Generate Single Sex Offspring

In some instances, sequence complementarity facilitates base-pairing of one or more expression cassettes carrying trans-splicing constructs described herein to a target pre-mRNA. Although RNA trans-splicing can be a low frequency event, several modifications can be undertaken to increase its efficiency (see Reidmayr, L. et al. 2020. Methods Mol Biol. (2020). 2079:219-232).

In one aspect, trans-splicing involves spliceosome-mediated RNA trans-splicing (“SMART”) wherein an antisense RNA sequence may complex with a target intron by Watson-Crick base pairing. In one aspect, trans-splicing involves CRISPR Assisted RNA Fragment Trans-splicing (“CRAFT”) wherein Cas13 systems, comprising orthologs thereof such as RfxCas13d, assist the trans-splicing of exogenous RNA fragments into an endogenous pre-mRNA transcript. Detailed SMART and CRAFT trans-splicing processes are described, for example, in Fiflis, David N et al. “Repurposing CRISPR-Cas13 systems for robust mRNA trans-splicing.” Nature communications vol. 15,1 2325. 14 Mar. 2024, doi: 10.1038/s41467-024-46172-4; which is hereby incorporated by reference in its entirety. In one aspect, trans-splicing involves Programmable RNA Editing & Cleavage for Insertion, Substitution, and Erasure (“PRECISE”) wherein 3′ trans-splicing employs a programmable RNase to separate cis exons from pre-mRNA, promoting trans-splicing of an engineered trans-template and wherein 5′ trans-splicing employes cleavage of the poly(A) sitail of the trans-template by either programmable RNases or engineered ribozymes. Detailed PRECISE trans-splicing process is described, for example, in Schmitt-Ulms, Cian et al. “Programmable RNA writing with trans-splicing.” bioRxiv: the preprint server for biology 2024.01.31.578223. 1 Feb. 2024, doi: 10.1101/2024.01.31.578223. Preprint; which is hereby incorporated by reference in its entirety. Thus, RNA trans-splicing provides an engineering tool to express the transgene or gene of interest.

In one aspect, the present disclosure provides methods and compositions for generation of single sex offspring in animals by utilizing RNA trans-splicing processes. In some embodiments, efficiency of RNA trans-splicing process can be improved by utilizing a protein intermediary, e.g., an RNA binding framework. In some embodiments, efficiency of RNA trans-splicing processes can be improved by utilizing a protein intermediary, e.g., an RNA binding framework, along with sequence complementarity. In one aspect, the present disclosure provides methods and compositions for generation of single sex offspring in animals by utilizing enhanced trans-splicing processes via an RNA binding framework to express two or more transgenes or genes of interest, e.g., a toxin, in which when expressed is lethal to the cell. In some embodiments, translation of a first transgenic protein of the two or more transgenic proteins is initiated when RNA trans-splicing occurs at both the first splice site (e.g., a 5′ splice site relative to a gene of interest; acceptor splice site) and the second splice site (e.g., a 3′ splice site relative to a gene of interest; donor splice site). In one aspect, the present disclosure provides methods and compositions to generate single sex offspring by utilizing enhanced trans-splicing process via an RNA binding framework.

In one aspect, a CRISPR/Cas system works as an RNA-guided, RNA-targeting viral defense system. In some instances, it comprises Higher Eukaryotes and Prokaryotes Nucleotide-binding (HEPN) endoRNase domains to cleave mRNA transcripts of invading viruses within bacteria and archaea. The RNA-targeting ability of the CRISPR/Cas system is used for targeted RNA editing in eukaryotes. The CRISPR/Cas RNA-targeting system allows targeting of nucleic acid fragments including RNA molecules. It permits cleaving RNAs in response to finding a target.

CRISPR/Cas systems can comprise class I and class II. Class I systems can use a complex of multiple Cas proteins to degrade foreign nucleic acids. Class II systems can use a single large Cas protein for the same purpose. Class I can be divided into types I, III, and IV. Class II can be divided into types II, V, and VI.

In some instances, the CRISPR/Cas system comprises a Cas polypeptide and an RNA binding protein (RBP). In some instances, the Cas polypeptide is linked to the RBP. In some instances, the Cas polypeptide is a Cas endonuclease. In some instances, the Cas endonuclease is a class I Cas endonuclease. In some instances, the Cas endonuclease is a class II Cas endonuclease. In some embodiments, Cas endonuclease is a type II, type III, or type VI Cas endonuclease. In some instances, the Cas endonuclease is a class II, type II Cas endonuclease. In some instances, the Cas endonuclease is a class II, type III Cas endonuclease. In some instances, the Cas endonuclease is a class II, type VI Cas endonuclease.

In some instances, the Cas endonuclease is an RNA-guided RNA endonuclease. In some embodiments, the Cas endonuclease comprises Cas9, Cas13, Csm/Cmr, Cas7-11, DisCas7-11, and Cas12a. In some embodiments, the Cas endonuclease is selected from the group consisting of Cas9, Cas13, Csm/Cmr, Cas7-11, DisCas7-11, and Cas12a. In some instances, the Cas endonuclease is Cas9. In some instances, the Cas endonuclease is Cas13. In some instances, the Cas endonuclease is Csm/Cmr. In some instances, the Cas endonuclease is Cas12a. In some instances, the Cas endonuclease is Cas7-11. In some instances, the Cas7-11 is Cas7-11a, Cas7-11b, Cas7-11c, or Cas7-11d. In some instances, the Cas7-11 is DisCas7-11. In some instances, the Cas7-11 is dCas7-11. In some instances, the dCas7-11 is dDisCas7-11.

In some instances, the RBP is MS2 coat protein (MCP). In some instances, the RBP is PP7 bacteriophage coat protein. In some instances, the RBP is small RNA phage PRR1. In some instances, the RBP is RNA bacteriophages QB coat protein.

In some instances, the activity of a CRISPR/Cas system is modified via a deactivated Cas endonuclease activity (dCas), which is understood to be interchangeably referred to as a dead Cas endonuclease activity. dCas proteins are Cas proteins devoid of nucleolytic activity. They can be used to deliver functional cargos to targeted sites in the genome.

In some instances, the Cas polypeptide is a variant of the Cas endonuclease. In some instances, the Cas polypeptide is an inactive form of the Cas endonuclease. In some instances, the Cas polypeptide binds to a polynucleotide (or nucleic acid sequence) but does not cleave the polynucleotide (or nucleic acid sequence). In some embodiments, the Cas polypeptide is a deactivated Cas13 (dCas13) or a deactivated dDisCas7-11 (dDisCas7-11).

In some embodiments, the Cas polypeptide is a dCas13a, dCas13b, dCas13c, or dCas13d. In some instances, the Cas polypeptide is a deactivated Cas13 (dCas13). In some instances, the Cas endonuclease is dCas9. In some instances, the Cas endonuclease is deactivated Csm/Cmr. In some instances, the Cas endonuclease is dCas12a. In some instances, the Cas polypeptide is dCas13a. In some instances, the Cas polypeptide is dCas13b. In some instances, the Cas polypeptide is dCas13c. In some instances, the Cas polypeptide is dCas13d. In some instances, the Cas polypeptide is a variant of a Prevotella sp. Cas13b (PspCas13b). In some instances, the Cas endonuclease is dCas7-11. In some instances, the dCas7-11 is dDisCas7-11.

In some instances, the CRISPR/Cas system comprises a trans-splicing replicon RNA (repRNA). In some instances, the repRNA encodes a transgenic protein, a splice site, an intron with RBP-binding hairpins, and a polyadenylation signal. In some instances, the repRNA encodes two or more transgenic proteins, two or more splice sites, and one or more introns. In some embodiments, a replicon RNA (repRNA) comprises a nucleic acid sequence encoding two or more transgenic proteins, two or more splice sites, and one or more introns, wherein the two or more transgenic proteins comprise a first transgenic protein and a second transgenic protein. In some embodiments, a 5′ end of the nucleic acid sequence encoding the first transgenic protein comprises a first splice site and a 3′ end of the nucleic acid sequence encoding the first transgenic protein comprises a second splice site. In some embodiments, the first transgenic protein is located on a 5′ end of the second transgenic protein. In some embodiments, the second transgenic protein is located on the 3′ end of the second splice site. In some embodiments, the first splice site is an acceptor splice site. In some embodiments, the second splice site is a donor splice site. In some embodiments, at least one of the one or more introns does not comprise a stop codon. In some embodiments, the one or more introns located between the nucleic acid sequence encoding the first transgenic protein and the second transgenic protein does not comprise a stop codon. In some embodiments, at least one of the one or more introns comprises a sequence that is complementary to one or more second sequence variants of the gene. In some embodiments, the one or more introns is connected to an RBP-binding hairpin. In some embodiments, the one or more introns does not comprise a stop codon. In some instances, the Cas polypeptide linked with the RBP recruits the trans-splicing replicon RNA (repRNA) and inhibits cis-splicing.

In some embodiments, the RBP is selected from the group consisting of MS2 coat protein (MCP), PP7 bacteriophage coat protein, small RNA phage PRR1, and RNA bacteriophages QB coat protein.

In some instances, the number of the RBP-binding hairpins is at least about 1. In some instances, the number of the RBP-binding hairpins is at least about 2. In some instances, the number of the RBP-binding hairpins is at least about 3. In some instances, the number of the RBP-binding hairpins is at least about 4. In some instances, the number of the RBP-binding hairpins is at least about 5. In some instances, the number of the RBP-binding hairpins is at least about 6. In some instances, the number of the RBP-binding hairpins is at least about 7. In some instances, the number of the RBP-binding hairpins is at least about 8. In some instances, the number of the RBP-binding hairpins is at least about 9. In some instances, the number of the RBP-binding hairpins is at least about 10. In some instances, the number of the RBP-binding hairpins is at least about 12. In some instances, the number of the RBP-binding hairpins is at least about 15. In some instances, the number of the RBP-binding hairpins is at least about 20. In some instances, the number of the RBP-binding hairpins is at most 2, at most 3, at most 4, at most 5, at most 6, at most 7, at most 8, at most 9, at most 10, at most 11, at most 12, at most 13, at most 14, at most 15, at most 16, at most 17, at most 18, at most 19, or at most 20. In some instances, the number of the RBP-binding hairpins is 1, 2, 3, 4, 5, 6, 7, 8, 9, 10, 11, 12, 13, 14, 15, 16, 17, 18, 19, or 20.

In some embodiments, the one or more CRISPR RNA-guided complexes comprise the guide RNA (gRNA), the Cas polypeptide, the repRNA, or a combination thereof.

In some instances, the CRISPR/Cas system comprises a guide RNA that directs sequence specific binding of one or more CRISPR RNA-guided complexes. A gRNA can comprise an RNA that functions as a guide for a Cas polypeptide, with which it forms complexes. A gRNA targets the complementary sequences of a target genome by base pairing. A gRNA can comprise a spacer sequence that is complementary to a corresponding target nucleic acid sequence, referred to as a protospacer. The term “spacer sequence” can include any polynucleotide having sufficient complementarity with a target nucleic acid sequence (i.e., “protospacer”) to hybridize with the target nucleic acid sequence and direct sequence-specific binding of an effector complex (e.g., a CRISPR RNA-guided complex) to the target sequence. In some instances, a gRNA comprises a spacer sequence and a scaffold sequence. A scaffold sequence can be a hairpin structure. In some cases, the scaffold sequence is downstream of the spacer sequence.

### Expression Cassette

In one aspect, the present disclosure provides one or more expression cassettes for generating single sex offspring. In some embodiments, one or more expression cassettes comprise a nucleic acid sequence encoding a replicon RNA (repRNA), comprising a nucleic acid sequence encoding two or more transgenic proteins, two or more splice sites, and one or more introns, wherein the two or more transgenic proteins comprise a first transgenic protein and a second transgenic protein. In some embodiments, a 5′ end of the nucleic acid sequence encoding the first transgenic protein comprises a first splice site and a 3′ end of the nucleic acid sequence encoding the first transgenic protein comprises a second splice site. In some embodiments, the first transgenic protein is located on a 5′ end of the second transgenic protein. In some embodiments, the second transgenic protein is located on the 3′ end of the second splice site. In some embodiments, the first splice site is an acceptor splice site. In some embodiments, the second splice site is a donor splice site. In some embodiments, at least one of the one or more introns does not comprise a stop codon. In some embodiments, the one or more introns located between the nucleic acid sequence encoding the first transgenic protein and the second transgenic protein does not comprise a stop codon. In some embodiments, at least one of the one or more introns comprises a sequence that is complementary to one or more second sequence variants of the gene.

In some embodiments, one or more expression cassettes comprise a nucleic acid sequence encoding a replicon RNA (repRNA), comprising a nucleic acid sequence encoding two or more transgenic proteins, two or more splice sites, and one or more introns, wherein the two or more transgenic proteins comprise a first transgenic protein and a second transgenic protein; wherein a 5′ end of the nucleic acid sequence encoding the first transgenic protein comprises a first splice site and a 3′ end of the nucleic acid sequence encoding the first transgenic protein comprises a second splice site; wherein at least one of the one or more introns does not comprise a stop codon; and wherein at least one of the one or more introns comprises a sequence that is complementary to one or more second sequence variants of the gene. In some embodiments, one or more expression cassettes comprise a nucleic acid sequence encoding a replicon RNA (repRNA), comprising a nucleic acid sequence encoding two or more transgenic proteins, two or more splice sites, and one or more introns, wherein the two or more transgenic proteins comprise a first transgenic protein and a second transgenic protein; wherein a 5′ end of the nucleic acid sequence encoding the first transgenic protein comprises a first splice site and a 3′ end of the nucleic acid sequence encoding the first transgenic protein comprises a second splice site; wherein the first transgenic protein is located on a 5′ end of the second transgenic protein; wherein at least one of the one or more introns does not comprise a stop codon; and wherein at least one of the one or more introns comprises a sequence that is complementary to one or more second sequence variants of the gene.

In some embodiments, the one or more expression cassettes further comprises one or more nucleic acid sequence encoding a protein intermediary, e.g., an RNA binding framework and a replicon RNA (repRNA). In some embodiments, the repRNA comprises an RNA trans-splicing construct. In some embodiments, the protein intermediary, e.g., an RNA binding framework, comprises a CRISPR/Cas system and a guide RNA (gRNA). In some embodiments, the CRISPR/Cas system comprises a Cas polypeptide linked to an RNA Binding Protein (RBP). In some embodiments, the one or more expression cassettes further comprises a second nucleic acid sequence encoding a Cas polypeptide linked to an RNA Binding Protein (RBP) and a third nucleic acid sequence encoding a guide RNA (gRNA) capable of directing one or more CRISPR RNA-guided complexes encoded by the one or more expression cassettes to one or more second sequence variants of the gene.

In some embodiments, the repRNA comprises a nucleic acid sequence encoding two or more transgenic proteins (or transgenes), two or more splice sites, and one or more introns. In some embodiments, the repRNA comprises a nucleic acid sequence encoding two or more transgenic proteins (or transgenes), two or more splice sites, one or more introns, and one or more RBP-binding hairpins. In some embodiments, the repRNA comprises a nucleic acid sequence encoding two or more transgenic proteins, two or more splice sites, and one or more introns, wherein each side of at least one of the two or more transgenic proteins is flanked by a first splice site and a second splice site of the two or more splice sites; wherein at least one of the one or more introns (a) is connected to an RBP-binding hairpin, and/or (b) does not comprise a stop codon. In some embodiments, the two or more transgenic proteins (or transgenes) provide a safety switch to differentiate between on-target and off-target trans-splicing events.

In some embodiments, the one or more introns comprise a first intron located on the 5′ end of the first transgenic protein, and wherein the first intron comprises a first RBP-binding hairpin. In some embodiments, the one or more introns comprise a second intron located on the 3′ end of the first transgenic protein, and wherein the second intron comprises a second RBP-binding hairpin. In some embodiments, a second RBP-binding hairpin is on a nucleic acid sequence located on a 3′ end of the second transgenic protein. In some embodiments, a second RBP-binding hairpin is on a nucleic acid sequence located on the 5′ end of the second transgenic protein. In some embodiments, the one or more introns comprises a first intron located on the 3′ end of the first transgenic protein, and wherein the first intron comprises a first RBP-binding hairpin.

In some instances, provided herein are expression cassettes comprising nucleotide sequences encoding two or more transgenic proteins, two or more splice sites, one or more introns to be expressed by a transfected cell. Provided herein are expression cassettes comprising nucleotide sequences encoding a transgene or gene of interest and regulatory sequence to be expressed by a transfected cell. In some embodiments, provided herein are expression cassettes comprising: (i) a first nucleic acid sequence encoding a Cas polypeptide linked to an RNA Binding Protein (RBP); (ii) a second nucleic acid sequence encoding a replicon RNA (repRNA), comprising a nucleic acid sequence encoding two or more transgenic proteins, two or more splice sites, and one or more introns, wherein each side of at least one of the two or more transgenic proteins is flanked by a first splice site and a second splice site of the two or more splice sites; wherein at least one of the one or more introns (a) is connected to an RBP-binding hairpin, and/or (b) does not comprise a stop codon; and (iii) a third nucleic acid sequence encoding a guide RNA (gRNA) capable of directing one or more CRISPR RNA-guided complexes encoded by the one or more expression cassettes to one or more second sequence variants of the gene. In some embodiments, one or more expression cassettes are used to generate an engineered animal. In some embodiments, the engineered animal includes, but is not limited to, cows, mice, rats, rabbits, guinea pigs, chicken, fish, birds, reptiles, camelids, bovine, chimpanzees, sheep, goats, and non-human primates.

As used herein, the terms “integration site” or “integrate” refer to the DNA constructs or vectors carrying one or more expression cassette(s) that are integrated into the chromosome in such a way that they are expressed and do not cause health issues for animal. In some instances, the one or more expression cassette(s) is integrated into one chromosome. In some instances, the one or more expression cassette(s) is integrated into both chromosomes. In some instances, the chromosome in which the one or more expression cassette(s) is integrated into is an allosome. In some instances, the chromosome in which the one or more expression cassette(s) is integrated into is an autosome.

In some embodiments, the one or more expression cassette(s) is integrated into a chromosome. In some embodiments, the chromosome is an autosome. In some embodiments, the chromosome is an allosome. In some embodiments, the one or more expression cassette(s) is integrated into both chromosomes. In some embodiments, the chromosomes are autosomes. In some embodiments, the one or more expression cassette(s) is integrated into an allosome.

As used herein, the terms “transgene” or “gene of interest” are used interchangeably to refer to a nucleotide sequence containing a gene sequence that has been isolated from one organism and is introduced into a different organism. In some instances, the transgene refers to an exogenous gene that is introduced into a cell or an organism by genetic engineering techniques. In some instances, the transgene is transferred into the target cell via a vector or expression cassette.

In some embodiments, provided herein are one or more expression cassettes comprising a nucleic acid sequence encoding a replicon RNA (repRNA), comprising a nucleic acid sequence encoding two or more transgenic proteins, two or more splice sites, and one or more introns, wherein the two or more transgenic proteins comprise a first transgenic protein and a second transgenic protein; wherein a 5′ end of the nucleic acid sequence encoding the first transgenic protein comprises a first splice site and a 3′ end of the nucleic acid sequence encoding the first transgenic protein comprises a second splice site; wherein at least one of the one or more introns does not comprise a stop codon; and wherein at least one of the one or more introns comprises a sequence that is complementary to one or more second sequence variants of the gene. In some embodiments, provided herein are one or more expression cassettes comprising a nucleic acid sequence encoding a replicon RNA (repRNA), comprising a nucleic acid sequence encoding two or more transgenic proteins, two or more splice sites, and one or more introns, wherein the two or more transgenic proteins comprise a first transgenic protein and a second transgenic protein; wherein a 5′ end of the nucleic acid sequence encoding the first transgenic protein comprises a first splice site and a 3′ end of the nucleic acid sequence encoding the first transgenic protein comprises a second splice site; wherein the first transgenic protein is located on a 5′ end of the second transgenic protein; wherein at least one of the one or more introns does not comprise a stop codon; and wherein at least one of the one or more introns comprises a sequence that is complementary to one or more second sequence variants of the gene. In some embodiments, the first transgenic protein is located on a 5′ end of the second transgenic protein. In some embodiments, the first splice site is an acceptor splice site. In some embodiments, the second splice site is a donor splice site.

In some embodiments, the one or more expression cassettes further comprises a second nucleic acid sequence encoding a Cas polypeptide linked to an RNA Binding Protein (RBP) and a third nucleic acid sequence encoding a guide RNA (gRNA) capable of directing one or more CRISPR RNA-guided complexes encoded by the one or more expression cassettes to one or more second sequence variants of the gene. In some embodiments, the one or more introns comprises a first intron located on the 5′ end of the first transgenic protein, and wherein the first intron comprises a first RBP-binding hairpin. In some embodiments, the one or more introns comprises a second intron located on the 3′ end of the first transgenic protein, and wherein the second intron comprises a second RBP-binding hairpin. In some embodiments, a second RBP-binding hairpin is on a nucleic acid sequence located on a 3′ end of the second transgenic protein. In some embodiments, a second RBP-binding hairpin is on a nucleic acid sequence located on the 5′ end of the second transgenic protein. In some embodiments, the one or more introns comprises a first intron located on the 3′ end of the first transgenic protein, and wherein the first intron comprises a first RBP-binding hairpin.

In some embodiments, translation of a first transgenic protein of the two or more transgenic proteins is initiated when RNA trans-splicing occurs at both the first splice site and the second splice site. In some embodiments, translation of the first transgenic protein, but not the second transgenic protein, is initiated when RNA trans-splicing occurs at both the first splice site and the second splice site. In some embodiments, translation of each of the first transgenic protein and the second transgenic protein is initiated when RNA trans-splicing occurs only at the first splice site.

In some embodiments, the two or more transgenic proteins comprises a first transgenic protein and a second transgenic protein. In some embodiments, the first transgenic protein is the same protein as the second transgenic protein. In some embodiments, the first transgenic protein is different than the second transgenic protein.

In some embodiments, the first transgenic protein is located on the 5′ end of the second transgenic protein. In some embodiments, the first transgenic protein is flanked by a splice site on each side. In some embodiments, the two or more splice sites comprises a first splice site and a second splice site. In some embodiments, the first splice site is an acceptor splice site. In some embodiments, the second splice site is a donor splice site.

In some embodiments, the first transgenic protein is a reporter gene. In some embodiments, the first transgenic protein is a toxin. In some embodiments, a transgene or gene of interest comprises protein-coding genes. In some instances, the protein-coding genes encode a toxin or toxic protein. In some instances, the protein-coding genes encode a toxin fragment. In some instances, the protein-coding genes encode a disease resistant protein. In some instances, the protein-encoding genes encode antimicrobial peptides. In some instances, a transgene or gene of interest comprises an engineered protein. In some embodiments, the engineered protein is a fusion protein. In some embodiments, the transgene or gene of interest comprises a full-length protein. In some embodiments, the transgene or gene of interest comprises a protein fragment. In some embodiments, the transgene or gene of interest comprises an active protein. In some embodiments, the transgene or gene of interest comprises an inactive protein or protein fragment. In some embodiments, the transgene or gene of interest comprises a toxin gene. In some embodiments, the transgene or gene of interest comprises a fluorescent protein. In some embodiments, the transgene or gene of interest comprises one or more of a green fluorescent protein (GFP), yellow fluorescent protein (YFP), red fluorescent protein (RFP), blue fluorescent protein (BFP), cyan fluorescent protein (CFP), and orange fluorescent protein (OFP).

In some embodiments, translation of the first transgenic protein and the second transgenic protein is initiated when RNA trans-splicing occurs only at the first splice site. In some embodiments, translation of the first transgenic protein, but not the second transgenic protein, is initiated when RNA trans-splicing occurs at both the first splice site and the second splice site.

As used herein, the terms “toxin” or “toxic protein” refer to any protein that is capable of killing or severely impairing the function of a cell. In some instances, the cell expressing functional toxin is lethal. For example, nuclease Barnase is bacterial protein that has ribonuclease activity. Nuclease Barnase can be a toxin and is lethal to the cell when expressed without its inhibitor, Barstar.

In some instances, the first transgenic protein is a toxin. In some embodiments, the first transgene encodes a toxin. In some embodiments, the toxin comprises nuclease, ribosome toxin, or protease. In some embodiments, the toxin comprises a nuclease or a protease. In some embodiments, the nuclease comprises Barnase, RNase, or restriction endonucleases. In some embodiments, the nuclease comprises a Barnase or an RNase. In some embodiments, the ribosome toxin comprises diphtheria, ricin, abrin, or pokeweed antiviral protein. In some embodiments, the protease comprises caspases, proteinase K, trypsin, chymotrypsin, or papain. Other toxins known in the art that are useful for killing a host cell, or an endogenous protein whose overexpression is cytotoxic can be used in the expression cassettes described herein.

As used herein, the term “cognate toxin inhibitor” refers to an inhibitor that inactivates a function of a toxin. For example, Barstar is a cognate toxin inhibitor to Barnase, a toxin. When both Barnase and Barstar are expressed in the same cell, the function of Barnase is inhibited by Barstar, therefore, the cell is viable.

In some embodiments, the first transgenic protein is located on a 5′ end of the second transgenic protein. In some embodiments, the second transgenic protein is a cognate toxin inhibitor. In some embodiments, the cognate toxin inhibitor inhibits function of the first transgenic protein, e.g., a toxin. In some embodiments, the first transgenic protein is a Barnase or a variant thereof, and the second transgenic protein is a Barstar or a variant thereof. In some embodiments, the Barnase or the variant thereof is modified so that the Barnase or the variant thereof are more hyperactive or more hypoactive as compared to an unmodified version. In some embodiments, the Barstar or the variant thereof is modified so that the Barstar or the variant thereof are more hyperactive or more hypoactive as compared to an unmodified version. In some embodiments, the unmodified version of the Barnase or the variant thereof is a wildtype. In some embodiments, the unmodified version of the Barstar or the variant thereof is a wildtype.

In some embodiments, the first transgenic protein is a protease, and the second transgenic protein is a protease inhibitor. In some embodiments, the first transgenic protein is a Barnase, and the second transgenic protein is a Barstar. In some embodiments, the first transgenic protein is an RNase, and the second transgenic protein is RNase inhibitor. In some embodiments, the first transgenic protein is a restriction endonuclease, and the second transgenic protein is a restriction endonuclease inhibitor. In some embodiments, the first transgenic protein is a diphtheria, and the second transgenic protein is a diphtheria antitoxin. In some embodiments, the first transgenic protein is a ricin, and the second transgenic protein is a ricin inhibitor. In some embodiments, the first transgenic protein is an abrin, and the second transgenic protein is an abrin inhibitor. In some embodiments, the first transgenic protein is a pokeweed antiviral protein, and the second transgenic protein is a pokeweed antiviral protein inhibitor. In some embodiments, the first transgenic protein is a caspase, and the second transgenic protein is a caspase inhibitor. In some embodiments, the first transgenic protein is a chymotrypsin, and the second transgenic protein is a chymotrypsin inhibitor. In some embodiments, the first transgenic protein is a papain, and the second transgenic protein is a papain inhibitor. In some embodiments, (i) the first transgenic protein is a restriction endonuclease, and the second transgenic protein is a restriction endonuclease inhibitor; (ii) the first transgenic protein is a diphtheria, and the second transgenic protein is a diphtheria antitoxin; (iii) the first transgenic protein is a ricin, and the second transgenic protein is a ricin inhibitor; (iv) the first transgenic protein is an abrin, and the second transgenic protein is an abrin inhibitor; (v) the first transgenic protein is a pokeweed antiviral protein, and the second transgenic protein is a pokeweed antiviral protein inhibitor; (vi) the first transgenic protein is a caspase, and the second transgenic protein is a caspase inhibitor; (vii) the first transgenic protein is a trypsin, and the second transgenic protein is a trypsin inhibitor; (viii) the first transgenic protein is a chymotrypsin, and the second transgenic protein is a chymotrypsin inhibitor; or (ix) the first transgenic protein is a papain, and the second transgenic protein is a papain inhibitor. Other pairs of toxin and cognate toxin inhibitor can be used with the expression cassettes, compositions, or methods described herein.

In some embodiments, the first transgenic protein and the second transgenic protein have the same open reading frame. In some embodiments, the first transgenic protein and the second transgenic protein are expressed together. In some embodiments, the second transgenic protein is not expressed without the presence of the first transgenic protein on the same mRNA. In some embodiments, the first transgenic protein and the second transgenic protein are expressed or translated on the same polypeptide.

In some embodiments, the nucleic acid sequence encoding the first transgenic protein does not contain a start codon.

As used herein, the term “intron” refers to a section of pre-mRNA that is removed via splicing and is not encoded in the translated protein. In some aspects, the intron encodes sequences that facilitate gene expression.

In some embodiments, the one or more expression cassettes further comprise one or more introns. In some aspects, the one or more introns encodes sequences that facilitate the gene expression. In some instances, the one or more introns facilitates RNA trans-splicing process. In some embodiments, the one or more introns is a naturally occurred intron encoded in the gene. In some embodiments, the one or more introns is an engineered intron. In some embodiments, the engineered intron is placed at the 5′ end of the open reading frame of the DNA construct. In some embodiments, the intron is placed at the 3′ end of the mRNA to increase mRNA stability. In some embodiments, the intron comprises an AU-rich element that is placed at the 3′ end of the mRNA.

In some embodiments, at least one of the one or more introns comprises a sequence that is complementary to one or more second sequence variants of the gene. In some embodiments, the sequence that is complementary to one or more second sequence variants of the gene is designed for sequence complementarity to facilitate base-pairing of one or more expression cassettes carrying trans-splicing constructs described herein to a target pre-mRNA.

In some embodiments, the one or more introns located between nucleic acid sequence encoding two or more transgenic proteins does not comprise a stop codon. In some embodiments, the one or more introns located between nucleic acid sequence encoding a first transgenic protein and a second transgenic protein does not comprise a stop codon.

In some embodiments, the one or more introns comprises a first intron located on the 5′ end of the first transgenic protein, and wherein the first intron comprises a first RBP-binding hairpin. In some embodiments, the one or more introns comprises a second intron located on the 3′ end of the first transgenic protein, and wherein the second intron comprises a second RBP-binding hairpin. In some embodiments, a second RBP-binding hairpin is on a nucleic acid sequence located on a 3′ end of the second transgenic protein. In some embodiments, a second RBP-binding hairpin is on a nucleic acid sequence located on the 5′ end of the second transgenic protein. In some embodiments, the one or more introns comprises a first intron located on the 3′ end of the first transgenic protein, and wherein the first intron comprises a first RBP-binding hairpin.

In some embodiments, a first intron of the one or more introns comprises a first RBP-binding hairpin. In some embodiments, the first RBP-binding hairpin is located on the 5′ end of the nucleic acid sequence encoding the first transgenic protein.

In some embodiments, a last intron of the one or more introns comprises a second RBP-binding hairpin. In some embodiments, a second RBP-binding hairpin is on nucleic acid sequence located on the 3′ end of the first transgenic protein. In some embodiments, a second RBP-binding hairpin is on nucleic acid sequence located on the 3′ end of the second transgenic protein. In some embodiments, a second RBP-binding hairpin is on nucleic acid sequence located on the 5′ end of the second transgenic protein. In some embodiments, a second RBP-binding hairpin is on an intron located between nucleic acid sequences encoding the first transgenic protein and the second transgenic protein.

In some embodiments, a combination of a sequence complementarity and a protein intermediary, e.g., an RNA binding framework such as CRISPR/Cas system and RBP binding hairpin, is utilized in the trans-splicing process. In some embodiments, provided herein are one or more expression cassettes comprising (i) a first nucleic acid sequence encoding a Cas polypeptide linked to an RNA Binding Protein (RBP); (ii) a second nucleic acid sequence encoding a replicon RNA (repRNA), comprising a nucleic acid sequence encoding two or more transgenic proteins, two or more splice sites, and one or more introns, wherein the two or more transgenic proteins comprise a first transgenic protein and a second transgenic protein; and (iii) a third nucleic acid sequence encoding a guide RNA (gRNA) capable of directing one or more CRISPR RNA-guided complexes encoded by the one or more expression cassettes to one or more third sequence variants of the gene, wherein the one or more second sequence variants of the gene and the one or more third sequence variants of the gene do not have sequence identity to each other. In some embodiments, a 5′ end of the nucleic acid sequence encoding the first transgenic protein comprises a first splice site and a 3′ end of the nucleic acid sequence encoding the first transgenic protein comprises a second splice site. In some embodiments, the first transgenic protein is located on a 5′ end of the second transgenic protein. In some embodiments, at least one of the one or more introns is connected to an RBP-binding hairpin. In some embodiments, at least one of the one or more introns does not comprise a stop codon. In some embodiments, at least one of the one or more introns comprises a sequence that is complementary to one or more second sequence variants of the gene.

In some embodiments, provided herein are one or more expression cassettes comprising (i) a first nucleic acid sequence encoding a Cas polypeptide linked to an RNA Binding Protein (RBP); (ii) a second nucleic acid sequence encoding a replicon RNA (repRNA), comprising a nucleic acid sequence encoding two or more transgenic proteins, two or more splice sites, and one or more introns, wherein the two or more transgenic proteins comprise a first transgenic protein and a second transgenic protein; wherein a 5′ end of the nucleic acid sequence encoding the first transgenic protein comprises a first splice site and a 3′ end of the nucleic acid sequence encoding the first transgenic protein comprises a second splice site; and wherein at least one of the one or more introns (a) is connected to an RBP-binding hairpin, and/or (b) does not comprise a stop codon; wherein at least one of the one or more introns comprises a sequence that is complementary to one or more second sequence variants of the gene; and (iii) a third nucleic acid sequence encoding a guide RNA (gRNA) capable of directing one or more CRISPR RNA-guided complexes encoded by the one or more expression cassettes to one or more third sequence variants of the gene, wherein the one or more second sequence variants of the gene and the one or more third sequence variants of the gene do not have sequence identity to each other.

As used herein, the term “promoter” refers to a section of DNA to which proteins, e.g., transcription factors, bind and induce transcription of the adjacent gene located downstream of the promoter. In some instances, promoters are more active or less active, e.g., driving more transcription or less transcription of the downstream gene either based on their intrinsic strength as a promoter or in response to various signaling events. In some instances, promoters are active at certain times during development, e.g., during embryogenesis or early development. In some instances, promoters are active in certain cell types, e.g., hematopoietic progenitor cells. In some instances, proteins, e.g., transcription factors, can be conditionally recruited to a promoter region to increase transcription or decrease transcription of the downstream gene.

In some embodiments, the one or more expression cassettes in the non-human vertebrate animal further comprises a promoter. In some embodiments, the one or more expression cassettes comprise a promoter that is expressed during embryogenesis. In some embodiments, the one or more expression cassettes comprises an inducible promoter. In some embodiments, the promoter is inactive in the adult non-human vertebrate animal. In some embodiments, the promoter is active during embryogenesis. In some embodiments, the promoter is active during embryogenesis and is silent or suppressed after embryogenesis. In some embodiments, the promoter is active during early development. In some embodiments, the promoter is activated by a transcription factor. In some embodiments, the transcription factor comprises a small molecule. In some embodiments, the small molecule comprises a tetracycline compound.

In some embodiments, the promoter is normally active in the adult non-human vertebrate animal. In some embodiments, the promoter is inactive during embryogenesis. In some embodiments, the promoter is active in a wide range of cell types. In some embodiments, the promoter is active in a specific cell type.

In some embodiments, the promoter is a constitutive promoter, e.g., ovalbumin gene promoter, chicken β-actin, cytomegalovirus (CMV) enhancer (CCAG or CAG promoter), histone H4 promoter, phosphoglycerol kinase (PGK) promoter, or other constitutive promoters. In some embodiments, the promoter is an inducible promoter system, e.g., temperature-inducible gene regulation (TIGR system) or tetracycline-controlled inducible operator system.

As used herein, the terms “trans-splicing acceptor gene” or “tsAG” refer to pre-mRNA that is expressed endogenously in the non-human vertebrate animal and is the target for RNA trans-splicing process. After RNA trans-splicing process, this tsAG is linked with a transgene or gene of interest from the trans-splicing donor gene (see below), resulting in a fusion mRNA molecule. After translation of the fusion mRNA molecule, a protein encoded by the transgene or gene of interest is expressed in the cell.

As used herein, the terms “trans-splicing donor gene” or “tsDG” refer to a pre-trans-splicing molecule (PTM) or an RNA-trans-splicing molecule (RTM) carrying a transgene or gene of interest to be expressed by joining the exons of the tsAG after the RNA trans-splicing process. In some instances, tsDGs are expressed from the expression cassette described in the present disclosure. In some instances, tsDGs are synthetic RNA that is introduced into the cell via other techniques, e.g., electroporation, etc.

In some aspects, the one or more expression cassettes comprise a nucleotide sequence that base pairs with the target region of the pre-mRNA of the tsAG in the wildtype genome of the non-human vertebrate animal. In some embodiments, the nucleotide sequence binds complementary to the target region of the pre-mRNA of the tsAG in the wildtype genome, thereby RNA trans-splicing process occurs. In some embodiments, the RNA trans-splicing is a 5′-trans-splicing. In some embodiments, the RNA trans-splicing is a 3′-trans-splicing. In some embodiments, the RNA trans-splicing is both a 5′ trans-splicing and a 3′ trans-splicing, thereby resulting in an internal exon replacement or insertion of a transgene. In some embodiments, the RNA trans-splicing is an internal exon replacement. In some embodiments, RNA trans-splicing process is spliceosome mediated. In some embodiments, the RNA trans-splicing process is ribozyme mediated.

In some instances, translation of a first transgenic protein of the two or more transgenic proteins is initiated when RNA trans-splicing occurs at both the first splice site and the second splice site. In some embodiments, the first splice site is an acceptor splice site. In some embodiments, the second splice site is a donor splice site. In some embodiments, the two or more transgenic proteins comprises a first transgenic protein and a second transgenic protein. In some embodiments, the first transgenic protein is located on a 5′ end of the second transgenic protein. In some embodiments, the first transgenic protein is flanked by the two or more splice sites. In some embodiments, translation of the first transgenic protein and the second transgenic protein is initiated when RNA trans-splicing occurs only at the first splice site. In some embodiments, translation of the first transgenic protein, but not the second transgenic protein, is initiated when RNA trans-splicing occurs at both the first splice site and the second splice site. In some embodiments, translation of each of the first transgenic protein and the second transgenic protein is initiated when RNA trans-splicing occurs only at the first splice site.

In some embodiments, the trans-splicing process occurs with a pre-mRNA encoded by a gene on an autosome. In some embodiments, the trans-splicing process occurs with a pre-mRNA encoded by a gene on an allosome.

In some embodiments, the trans-splicing process occurs with a pre-mRNA encoded by a gene on an autosome, and the one or more expression cassettes is integrated into an allosome. In some embodiments, the trans-splicing process occurs with a pre-mRNA encoded by a gene on an allosome, and the one or more expression cassettes is integrated into an allosome.

In some embodiments, the gene is a non-essential gene. In some embodiments, the gene is an essential gene. In some embodiments, the gene is expressed in an embryo. In some embodiments, the gene is a housekeeping gene that is constitutively expressed. In some embodiments, the gene is expressed only in an embryo. In some embodiments, the gene is not expressed in adult.

In some embodiments, the trans-splicing process occurs with a pre-mRNA encoded by a trans-splicing acceptor gene (tsAG). In some embodiments, the gene is a trans-splicing acceptor gene (tsAG).

In some embodiments, the target region for the trans-splicing event is in introns. In some embodiments, the target region in the introns is between exons of the pre-mRNA of the tsAG, thereby a protein encoded by an exon of the transgene from the tsDG is in frame for protein expression after the RNA trans-splicing. In some embodiments, the RNA trans-splicing is a 5′-trans-splicing. In some embodiments, the RNA trans-splicing is a 3′-trans-splicing. In some embodiments, the RNA trans-splicing is both a 5′ trans-splicing and a 3′ trans-splicing, thereby resulting in an internal exon replacement or insertion of a nucleic acid sequence encoding the first transgenic protein. In some embodiments, the RNA trans-splicing is both a 5′ trans-splicing and a 3′ trans-splicing, thereby resulting in an internal exon replacement or insertion of a nucleic acid sequence encoding only the first transgenic protein without the second transgenic protein.

In some aspects, the one or more expression cassettes comprise a splice site for the RNA trans-splicing process. In some embodiments, the splice site comprises an acceptor splice site (ass), a donor splice site (dss), or a combination thereof. In some embodiments, the splice site is located at the 5′ end of the transgene. In some embodiments, the splice site is located at the 3′ end of the transgene.

In some embodiments, a 5′ end of the nucleic acid sequence encoding the first transgenic protein comprises a first splice site and a 3′ end of the nucleic acid sequence encoding the first transgenic protein comprises a second splice site. In some instances, translation of the first transgenic proteins is initiated when RNA trans-splicing occurs at both the first splice site and the second splice site. In some embodiments, translation of the first transgenic protein, but not the second transgenic protein, is initiated when RNA trans-splicing occurs at both the first splice site and the second splice site. In some embodiments, the first splice site is an acceptor splice site. In some embodiments, the second splice site is a donor splice site. In some embodiments, translation of each of the first transgenic protein and the second transgenic protein is initiated when RNA trans-splicing occurs only at the first splice site.

As used herein, the terms “open reading frame” or “ORF” refer to a portion of a DNA or RNA sequence that encodes for a protein. In some instances, the DNA or RNA portion of the ORF does not contain stop codon. In some instances, ORF on the expression cassette carries nucleotide sequence encoding a protein from the transgene.

As used herein, the term “transcription terminator” or “terminator sequence” refer to a region of nucleic acid sequence that marks the end of a gene during transcription. In some instances, this region mediates transcriptional termination by triggering the release of transcript RNA from the translational complex. In some instances, the transcription terminator involves direct activity of termination factors. In some instances, the transcription terminator involves indirect activity of termination factors.

In some embodiments, the one or more expression cassettes in the non-human vertebrate animal further comprise a transcription terminator. In some embodiments, the transcription terminator comprises poly-A signals. In some embodiments, the terminator sequences comprise sequence motif AAUAAA. In some embodiments, the terminator sequences comprise mammalian terminators, e.g., SV40, hGH, BGH, and rbGlob. It is envisioned that other terminator sequences or motifs can also be used.

In some embodiments, the one or more expression cassettes in the non-human vertebrate animal comprises one or more expression cassettes comprising: a nucleic acid sequence encoding a replicon RNA (repRNA). In some embodiments, the repRNA comprises a nucleic acid sequence encoding two or more transgenic proteins, two or more splice sites, and one or more introns, wherein the two or more transgenic proteins comprise a first transgenic protein and a second transgenic protein. In some embodiments, a 5′ end of the nucleic acid sequence encoding the first transgenic protein comprises a first splice site and a 3′ end of the nucleic acid sequence encoding the first transgenic protein comprises a second splice site. In some embodiments, at least one of the one or more introns does not comprise a stop codon. In some embodiments, at least one of the one or more introns comprises a sequence that is complementary to one or more second sequence variants of the gene.

In some embodiments, the one or more expression cassettes in the non-human vertebrate animal further comprise a nucleic acid encoding a Cas polypeptide and an RNA Binding Protein (RBP). In some embodiments, the one or more expression cassettes in the non-human vertebrate animal further comprise a guide RNA capable of directing sequence specific binding of one or more CRISPR RNA-guided complexes encoded by the one or more expression cassettes to targeted sites of genome. In some embodiments, the one or more expression cassettes further comprises a second nucleic acid sequence encoding a Cas polypeptide linked to an RNA Binding Protein (RBP) and a third nucleic acid sequence encoding a guide RNA (gRNA) capable of directing one or more CRISPR RNA-guided complexes encoded by the one or more expression cassettes to one or more second sequence variants of the gene. In some embodiments, the Cas polypeptide is linked to the RBP. In some embodiments, the one or more introns is connected to an RBP-binding hairpin. In some embodiments, the one or more introns does not comprise a stop codon.

In some embodiments, the one or more expression cassettes comprises a nucleic acid sequence encoding a replicon RNA (repRNA), comprising a nucleic acid sequence encoding two or more transgenic proteins, two or more splice sites, and one or more introns, wherein the two or more transgenic proteins comprise a first transgenic protein and a second transgenic protein; wherein a 5′ end of the nucleic acid sequence encoding the first transgenic protein comprises a first splice site and a 3′ end of the nucleic acid sequence encoding the first transgenic protein comprises a second splice site; wherein at least one of the one or more introns does not comprise a stop codon; and wherein at least one of the one or more introns comprises a sequence that is complementary to one or more second sequence variants of the gene. In some embodiments, one or more expression cassettes comprising: a nucleic acid sequence encoding a replicon RNA (repRNA), comprising a nucleic acid sequence encoding two or more transgenic proteins, two or more splice sites, and one or more introns, wherein the two or more transgenic proteins comprise a first transgenic protein and a second transgenic protein; wherein a 5′ end of the nucleic acid sequence encoding the first transgenic protein comprises a first splice site and a 3′ end of the nucleic acid sequence encoding the first transgenic protein comprises a second splice site; wherein the first transgenic protein is located on a 5′ end of the second transgenic protein; wherein at least one of the one or more introns does not comprise a stop codon; and wherein at least one of the one or more introns comprises a sequence that is complementary to one or more second sequence variants of the gene.

In some embodiments, the one or more expression cassettes comprises (i) a first nucleic acid sequence encoding a Cas polypeptide linked to an RNA Binding Protein (RBP); (ii) a second encoding two or more transgenic proteins, two or more splice sites, and one or more introns, wherein the two or more transgenic proteins comprise a first transgenic protein and a second transgenic protein; wherein a 5′ end of the nucleic acid sequence encoding the first transgenic protein comprises a first splice site and a 3′ end of the nucleic acid sequence encoding the first transgenic protein comprises a second splice site; and wherein at least one of the one or more introns (a) is connected to an RBP-binding hairpin, and/or (b) does not comprise a stop codon; and (iii) a third nucleic acid sequence encoding a guide RNA (gRNA) capable of directing one or more CRISPR RNA-guided complexes encoded by the one or more expression cassettes to one or more second sequence variants of the gene.

In some embodiments, one or more expression cassettes comprises (i) a first nucleic acid sequence encoding a Cas polypeptide linked to an RNA Binding Protein (RBP); (ii) a second nucleic acid sequence encoding a replicon RNA (repRNA), comprising a nucleic acid sequence encoding two or more transgenic proteins, two or more splice sites, and one or more introns, wherein the two or more transgenic proteins comprise a first transgenic protein and a second transgenic protein; wherein a 5′ end of the nucleic acid sequence encoding the first transgenic protein comprises a first splice site and a 3′ end of the nucleic acid sequence encoding the first transgenic protein comprises a second splice site; and wherein at least one of the one or more introns (a) is connected to an RBP-binding hairpin, and/or (b) does not comprise a stop codon; wherein at least one of the one or more introns comprises a sequence that is complementary to one or more second sequence variants of the gene; and (iii) a third nucleic acid sequence encoding a guide RNA (gRNA) capable of directing one or more CRISPR RNA-guided complexes encoded by the one or more expression cassettes to one or more third sequence variants of the gene, wherein the one or more second sequence variants of the gene and the one or more third sequence variants of the gene do not have sequence identity to each other.

Delivery of the DNA constructs carrying the one or more expression cassette(s) to generate an engineered animal, e.g., a chicken, is performed by viral transfection system, e.g., a lentiviral based system. Alternatively, a non-viral method is utilized. The non-viral method is based on genetically modified embryonic cells carrying a DNA construct to be transferred into the recipient embryo, thereby generating a transgenic/engineered animal, e.g., a chicken (see Bednarczyk, M. et al. 2018. 59:81-89).

In some embodiments, the method to generate engineered animals, e.g., chickens, comprises a viral transfection system. In some embodiments, the viral transfection system is a lentiviral based system. In various embodiments, the method to generate the engineered animal, e.g., a chicken, comprises a non-viral method, e.g., electroporation, lipofection, or CRISPR to transfer DNA construct into the targeted cell.

In another aspect, provided herein are one or more expression cassettes comprising a first nucleic acid sequence encoding a Cas polypeptide linked to an RNA Binding Protein (RBP); a second nucleic acid sequence encoding a replicon RNA (repRNA), comprising: a nucleic acid sequence encoding two or more transgenic proteins, wherein a first transgenic protein of the two or more transgenic proteins comprises a toxin, and wherein a second transgenic protein of the two or more transgenic proteins comprises a cognate toxin inhibitor, two or more splice sites, and one or more introns, wherein each side of the first transgenic protein is flanked by a first splice site and a second splice site of the two or more splice sites; wherein a first intron of the one or more introns is connected to an RBP-binding hairpin; wherein an intron located on nucleic acid sequence between the first transgenic protein and the second transgenic protein does not comprises a stop codon; and a third nucleic acid sequence encoding a guide RNA (gRNA) capable of directing one or more CRISPR RNA-guided complexes encoded by the one or more expression cassettes to one or more second sequence variants of the gene. In some embodiments, the one or more introns further comprises a last intron comprising a second RBP-binding hairpin. In some embodiments, an intron located on nucleic acid sequence between the first transgenic protein and the second transgenic protein further comprises a second RBP-binding hairpin.

### Modification of Intron to be Resistant to RNA Trans-Splicing

In some instances, the animal, e.g., a chicken, with one or more expression cassettes comprising an RNA trans-splicing construct carrying a transgene or gene of interest, e.g., a toxin, is also modified or engineered so that the RNA trans-splicing cannot occur in this animal. This engineered animal is used for breeding with a wildtype animal to generate single sex offspring.

In one aspect, provided herein is a non-human vertebrate animal having a modified genotype comprising one or more expression cassettes comprising trans-splicing constructs and a modified nucleic acid sequence that is not capable of the trans-splicing process. In some embodiments, the modified nucleic acid sequence in the non-human vertebrate animal having the modified genotype comprises one or more first sequence variants of a gene, wherein the one or more first sequence variants of the gene comprise a modified intron that is not capable of base pairing with a trans-splicing accepting gene.

In some embodiments, the trans-splicing accepting gene is a gene on an autosome. In some embodiments, the trans-splicing accepting gene is a gene on an allosome. In some embodiments, the one or more expression cassettes comprising trans-splicing constructs is designed for the trans-splicing process with a wildtype nucleic acid sequence of the gene. In some embodiments, the wildtype nucleic acid sequence of the gene comprises one or more second sequence variants of the gene. In some embodiments, the wildtype nucleic acid sequence of the gene further comprises one or more third sequence variants of the gene. In some embodiments, the one or more second sequence variants of the gene do not have sequence identity to the one or more first sequence variant of the gene. In some embodiments, the one or more first and second sequence variants of the gene do not have sequence identity to the one or more third sequence variants of the gene. In some embodiments, the one or more first sequence variants of the gene do not have sequence identity to the one or more third sequence variant of the gene. In some embodiments, the one or more second sequence variants of the gene and the one or more third sequence variants of the gene do not have sequence identity to each other.

In some embodiments, the nucleic acid sequence of the one or more expression cassettes cannot bind complementary to a mutated region of the pre-mRNA of the tsAG in an engineered animal, thereby RNA trans-splicing cannot occur. In some embodiments, provided herein is a non-human vertebrate animal having a modified genotype comprising one or more first sequence variants of a gene, wherein the one or more first sequence variants of the gene comprise a modified intron that is not capable of base pairing with a trans-splicing accepting gene and one or more expression cassettes comprising trans-splicing constructs.

In some instances, the engineered animal, e.g., a chicken, comprises a modified genotype with one or more first sequence variants of the gene having a modified intron that is not capable of base pairing with a trans-splicing accepting gene. In some instances, the engineered animal, e.g., a chicken, further comprises one or more expression cassettes comprising an RNA trans-splicing construct as described herein. In some instances, the engineered animal is a non-human vertebrate animal, including but not limited to cow, mouse, rat, rabbit, guinea pig, chicken, fish, bird, reptile, camelid, bovine, chimpanzee, sheep, goat, and non-human primate.

In some instances, the engineered animal, e.g., a chicken, comprises one or more expression cassette comprising an RNA trans-splicing construct as described herein. In some instances, the engineered animal, e.g., a chicken, further comprises a modified genotype with one or more first sequence variants of the gene having a modified intron that is not capable of base pairing with a trans-splicing accepting gene. In some instances, the engineered animal is a non-human vertebrate animal, including but not limited to cow, mouse, rat, rabbit, guinea pig, chicken, fish, bird, reptile, camelid, bovine, chimpanzee, sheep, goat, and non-human primate.

In some embodiments, the engineered animal, e.g., a chicken comprises one or more first sequence variants of a gene, wherein the one or more first sequence variants of the gene comprise a modified intron that is not capable of the RNA trans-splicing process. In some embodiments, the engineered animal, e.g., a chicken comprises one or more first sequence variants of a gene, wherein the one or more first sequence variants of the gene comprise a modified intron that is not capable of base pairing with a trans-splicing accepting gene.

In some instances, the modified intron is a mutated sequence located in introns. In some instances, the mutated sequence is located in 5′UTR. In some instances, the mutated sequence is located in 3′UTR. In some instances, the mutated sequence is not located in exons. In some instances, the mutated sequence is a naturally occurring variant. In some instances, the mutated sequence is generated via genetic engineered tools, e.g., CRISPR-Cas9 system or zinc-finger nucleases (ZFNs). Other engineering tools to mutate nucleotide sequence can be applied to this present disclosure. In some cases, the gene with the modified intron is a Rictor gene. Further, this RNA trans-splicing system for generation of single sex offspring can be applied to other animals, all of which are compatible with methods of the present disclosure and contemplated herein. Examples of animal include, but are not limited to cow, mouse, rat, rabbit, guinea pig, chicken, fish, bird, reptile, camelid, bovine, chimpanzee, sheep, goat, and non-human primate.

In some instances, the guide RNA directs sequence specific binding of one or more CRISPR RNA-guided complexes encoded by the one or more expression cassettes to one or more second sequence variants of the gene. In some embodiments, the one or more second sequence variants of the gene do not have sequence identity to the one or more first sequence variant of the gene. In some embodiments, the one or more second sequence variants of the gene have sequence identity to a wildtype sequence of the gene.

In some embodiments, the guide RNA (gRNA) is capable of directing one or more CRISPR RNA-guided complexes to one or more second sequence variants of the gene. In some embodiments, a guide RNA (gRNA) is capable of directing one or more CRISPR RNA-guided complexes encoded by the one or more expression cassettes to one or more second sequence variants of the gene. In some embodiments, the guide RNA (gRNA) is capable of directing one or more CRISPR RNA-guided complexes to one or more third sequence variants of the gene. In some embodiments, a guide RNA (gRNA) is capable of directing one or more CRISPR RNA-guided complexes encoded by the one or more expression cassettes to one or more third sequence variants of the gene.

### Animals for Generation of Single Sex Offspring Via RNA Trans-Splicing Methods

In an aspect, provided herein are non-human vertebrate animals having a modified genotype comprising: (1) one or more first sequence variants of a gene, wherein the one or more first sequence variants of the gene comprise a modified intron that is not capable of base pairing with a trans-splicing accepting gene; and (2) one or more expression cassettes comprising: a nucleic acid sequence encoding a replicon RNA (repRNA), comprising a nucleic acid sequence encoding two or more transgenic proteins, two or more splice sites, and one or more introns, wherein the two or more transgenic proteins comprise a first transgenic protein and a second transgenic protein; wherein a 5′ end of the nucleic acid sequence encoding the first transgenic protein comprises a first splice site and a 3′ end of the nucleic acid sequence encoding the first transgenic protein comprises a second splice site; wherein at least one of the one or more introns does not comprise a stop codon; and wherein at least one of the one or more introns comprises a sequence that is complementary to one or more second sequence variants of the gene. In some embodiments, translation of the first transgenic protein, but not the second transgenic protein, is initiated when RNA trans-splicing occurs at both the first splice site and the second splice site. In some embodiments, translation of each of the first transgenic protein and the second transgenic protein is initiated when RNA trans-splicing occurs only at the first splice site. In some embodiments, the first transgenic protein is located on a 5′ end of the second transgenic protein. In some embodiments, the first splice site is an acceptor splice site. In some embodiments, the second splice site is a donor splice site.

In some embodiments, the first transgenic protein is different than the second transgenic protein. In some embodiments, the first transgenic protein is a reporter gene. In some embodiments, the first transgenic protein is a toxin. In some embodiments, the toxin comprises a nuclease or a protease. In some embodiments, the nuclease comprises a Barnase or an RNase. In some embodiments, the protease comprises a caspase, trypsin, chymotrypsin, or papain. In some embodiments, the second transgenic protein is a cognate toxin inhibitor. In some embodiments, the first transgenic protein is a Barnase or a variant thereof, and the second transgenic protein is a Barstar or a variant thereof. In some embodiments, the first transgenic protein is an RNase, and the second transgenic protein is an RNase inhibitor. In some embodiments, the first transgenic protein is a restriction endonuclease, and the second transgenic protein is In some embodiments, (i) the first transgenic protein is a restriction endonuclease, and the second transgenic protein is a restriction endonuclease inhibitor; (ii) the first transgenic protein is a diphtheria, and the second transgenic protein is a diphtheria antitoxin; (iii) the first transgenic protein is a ricin, and the second transgenic protein is a ricin inhibitor; (iv) the first transgenic protein is an abrin, and the second transgenic protein is an abrin inhibitor; (v) the first transgenic protein is a pokeweed antiviral protein, and the second transgenic protein is a pokeweed antiviral protein inhibitor; (vi) the first transgenic protein is a caspase, and the second transgenic protein is a caspase inhibitor; (vii) the first transgenic protein is a trypsin, and the second transgenic protein is a trypsin inhibitor; (viii) the first transgenic protein is a chymotrypsin, and the second transgenic protein is a chymotrypsin inhibitor; or (ix) the first transgenic protein is a papain, and the second transgenic protein is a papain inhibitor. Other pairs of toxin and cognate toxin inhibitor can be used with the expression cassettes, compositions, or methods described herein.

In some aspects, the one or more expression cassettes further comprises a second nucleic acid sequence encoding a Cas polypeptide linked to an RNA Binding Protein (RBP) and a third nucleic acid sequence encoding a guide RNA (gRNA) capable of directing one or more CRISPR RNA-guided complexes encoded by the one or more expression cassettes to one or more second sequence variants of the gene. In some embodiments, the one or more introns comprises a first intron located on the 5′ end of the first transgenic protein, and wherein the first intron comprises a first RBP-binding hairpin. In some embodiments, the one or more introns comprises a second intron located on the 3′ end of the first transgenic protein, and wherein the second intron comprises a second RBP-binding hairpin. In some embodiments, a second RBP-binding hairpin is on a nucleic acid sequence located on a 3′ end of the second transgenic protein. In some embodiments, a second RBP-binding hairpin is on a nucleic acid sequence located on the 5′ end of the second transgenic protein. In some embodiments, the one or more introns comprises a first intron located on the 3′ end of the first transgenic protein, and wherein the first intron comprises a first RBP-binding hairpin.

In some embodiments, the one or more expression cassettes comprise a promoter that is expressed during embryogenesis. In some embodiments, the one or more expression cassettes comprises an inducible promoter. In some embodiments, the gene is a non-essential gene. In some embodiments, the gene is an essential gene. In some embodiments, the gene is expressed in an embryo. In some embodiments, the gene is a housekeeping gene that is constitutively expressed. In some embodiments, the non-human vertebrate animal is selected from the group consisting of cow, mouse, rat, rabbit, guinea pig, chicken, fish, bird, reptile, camelid, bovine, chimpanzee, sheep, goat, and non-human primate.

In some embodiments, the one or more second sequence variants of the gene do not have sequence identity to the one or more first sequence variant of the gene. In some embodiments, the one or more second sequence variants of the gene have sequence identity to a wildtype sequence of the gene.

In some embodiments, the RNA trans-splicing process is spliceosome mediated. In some embodiments, the RNA trans-splicing process is ribozyme mediated. In some embodiments, the Cas polypeptide is a Cas endonuclease. In some embodiments, the Cas endonuclease is a class II Cas endonuclease. In some embodiments, the Cas endonuclease is a type II, type III, or type VI Cas endonuclease. In some embodiments, the Cas endonuclease is an RNA-guided RNA endonuclease. In some embodiments, the Cas endonuclease is selected from the group consisting of Cas9, Cas13, Csm/Cmr, Cas7-11, DisCas7-11, and Cas12a. In some embodiments, the Cas7-11 is Cas7-11a, Cas7-11b, Cas7-11c, or Cas7-11d. In some embodiments, the Cas polypeptide is a variant of the Cas endonuclease. In some embodiments, the Cas polypeptide is an inactive form of the Cas endonuclease. In some embodiments, the Cas polypeptide binds to a polynucleotide but does not cleave the polynucleotide. In some embodiments, the Cas polypeptide is a deactivated Cas13 (dCas13) or a deactivated dDisCas7-11 (dDisCas7-11). In some embodiments, the Cas polypeptide is a dCas13a, dCas13b, dCas13c, or dCas13d. In some embodiments, the Cas polypeptide is a variant of a Prevotella sp. Cas13b (PspCas13b).

In some embodiments, the RBP is selected from the group consisting of MS2 coat protein (MCP), PP7 bacteriophage coat protein, small RNA phage PRR1, and RNA bacteriophages QB coat protein. In some embodiments, the number of the RBP-binding hairpins is at least 1, at least 2, at least 3, at least 4, at least 5, at least 6, at least 7, at least 8, at least 9, or at least 10. In some embodiments, the one or more CRISPR RNA-guided complexes comprise the guide RNA, the Cas polypeptide, the repRNA, or a combination thereof. In some embodiments, the RNA trans-splicing process at the 3′ splicing site results in translation of both the first transgenic protein and the second transgenic protein. In some embodiments, the RNA trans-splicing process at both the 3′ splicing site and the 5′ splicing site results in translation of the first transgenic protein but not the second transgenic protein.

In some embodiments, the gene is an autosomal gene. In some embodiments, the gene is an allosomal gene. In some embodiments, the one or more expression cassettes is located on an autosomal chromosome. In some embodiments, the one or more expression cassettes is integrated into an autosome. In some embodiments, the one or more expression cassettes is located on an allosomal chromosome. In some embodiments, the one or more expression cassettes is integrated into an allosome.

In another aspect, provided herein are non-human vertebrate animals having a modified genotype comprising: (1) one or more first sequence variants of a gene, wherein the one or more first sequence variants of the gene comprise a modified intron that is not capable of base pairing with a trans-splicing accepting gene; and (2) one or more expression cassettes comprising: a nucleic acid sequence encoding a replicon RNA (repRNA), comprising a nucleic acid sequence encoding two or more transgenic proteins, two or more splice sites, and one or more introns, wherein the two or more transgenic proteins comprise a first transgenic protein and a second transgenic protein; wherein a 5′ end of the nucleic acid sequence encoding the first transgenic protein comprises a first splice site and a 3′ end of the nucleic acid sequence encoding the first transgenic protein comprises a second splice site; wherein the first transgenic protein is located on a 5′ end of the second transgenic protein; wherein at least one of the one or more introns does not comprise a stop codon; and wherein at least one of the one or more introns comprises a sequence that is complementary to one or more second sequence variants of the gene.

In various aspects, provided herein are non-human vertebrate animals having a modified genotype comprising: (1) one or more first sequence variants of a gene, wherein the one or more first sequence variants of the gene comprise a modified intron that is not capable of base pairing with a trans-splicing accepting gene; and (2) one or more expression cassettes comprising: (i) a first nucleic acid sequence encoding a Cas polypeptide linked to an RNA Binding Protein (RBP); (ii) a second nucleic acid sequence encoding a replicon RNA (repRNA), comprising a nucleic acid sequence encoding two or more transgenic proteins, two or more splice sites, and one or more introns, wherein the two or more transgenic proteins comprise a first transgenic protein and a second transgenic protein; wherein a 5′ end of the nucleic acid sequence encoding the first transgenic protein comprises a first splice site and a 3′ end of the nucleic acid sequence encoding the first transgenic protein comprises a second splice site; and wherein at least one of the one or more introns (a) is connected to an RBP-binding hairpin, and/or (b) does not comprise a stop codon; and (iii) a third nucleic acid sequence encoding a guide RNA (gRNA) capable of directing one or more CRISPR RNA-guided complexes encoded by the one or more expression cassettes to one or more second sequence variants of the gene.

In another aspect, provided herein are non-human vertebrate animals having a modified genotype comprising: (1) one or more first sequence variants of a gene, wherein the one or more first sequence variants of the gene comprise a modified intron that is not capable of base pairing with a trans-splicing accepting gene; and (2) one or more expression cassettes comprising: (i) a first nucleic acid sequence encoding a Cas polypeptide linked to an RNA Binding Protein (RBP); (ii) a second nucleic acid sequence encoding a replicon RNA (repRNA), comprising a nucleic acid sequence encoding two or more transgenic proteins, two or more splice sites, and one or more introns, wherein the two or more transgenic proteins comprise a first transgenic protein and a second transgenic protein; wherein a 5′ end of the nucleic acid sequence encoding the first transgenic protein comprises a first splice site and a 3′ end of the nucleic acid sequence encoding the first transgenic protein comprises a second splice site; and wherein at least one of the one or more introns (a) is connected to an RBP-binding hairpin, and/or (b) does not comprise a stop codon; wherein at least one of the one or more introns comprises a sequence that is complementary to one or more second sequence variants of the gene; and (iii) a third nucleic acid sequence encoding a guide RNA (gRNA) capable of directing one or more CRISPR RNA-guided complexes encoded by the one or more expression cassettes to one or more third sequence variants of the gene, wherein the one or more second sequence variants of the gene and the one or more third sequence variants of the gene do not have sequence identity to each other.

In some embodiments, translation of the first transgenic protein, but not the second transgenic protein, is initiated when RNA trans-splicing occurs at both the first splice site and the second splice site. In some embodiments, translation of each of the first transgenic protein and the second transgenic protein is initiated when RNA trans-splicing occurs only at the first splice site. In some embodiments, the first transgenic protein is located on a 5′ end of the second transgenic protein. In some embodiments, the first splice site is an acceptor splice site. In some embodiments, the second splice site is a donor splice site. In some embodiments, the first transgenic protein is different than the second transgenic protein. In some embodiments, the first transgenic protein is a reporter gene.

In some embodiments, the first transgenic protein is a toxin. In some embodiments, the toxin comprises a nuclease or a protease. In some embodiments, the nuclease comprises a Barnase or an RNase. In some embodiments, the protease comprises a caspase, trypsin, chymotrypsin, or papain. In some embodiments, the second transgenic protein is a cognate toxin inhibitor. In some embodiments, the first transgenic protein is a Barnase or a variant thereof, and the second transgenic protein is a Barstar or a variant thereof. In some embodiments, the first transgenic protein is an RNase, and the second transgenic protein is an RNase inhibitor. In some embodiments, the first transgenic protein is a restriction endonuclease, and the second transgenic protein is In some embodiments, (i) the first transgenic protein is a restriction endonuclease, and the second transgenic protein is a restriction endonuclease inhibitor; (ii) the first transgenic protein is a diphtheria, and the second transgenic protein is a diphtheria antitoxin; (iii) the first transgenic protein is a ricin, and the second transgenic protein is a ricin inhibitor; (iv) the first transgenic protein is an abrin, and the second transgenic protein is an abrin inhibitor; (v) the first transgenic protein is a pokeweed antiviral protein, and the second transgenic protein is a pokeweed antiviral protein inhibitor; (vi) the first transgenic protein is a caspase, and the second transgenic protein is a caspase inhibitor; (vii) the first transgenic protein is a trypsin, and the second transgenic protein is a trypsin inhibitor; (viii) the first transgenic protein is a chymotrypsin, and the second transgenic protein is a chymotrypsin inhibitor; or (ix) the first transgenic protein is a papain, and the second transgenic protein is a papain inhibitor. Other pairs of toxin and cognate toxin inhibitor can be used with the expression cassettes, compositions, or methods described herein.

In other aspects herein, the one or more expression cassettes further comprises a second nucleic acid sequence encoding a Cas polypeptide linked to an RNA Binding Protein (RBP) and a third nucleic acid sequence encoding a guide RNA (gRNA) capable of directing one or more CRISPR RNA-guided complexes encoded by the one or more expression cassettes to one or more second sequence variants of the gene. In some embodiments, the one or more introns comprises a first intron located on the 5′ end of the first transgenic protein, and wherein the first intron comprises a first RBP-binding hairpin. In some embodiments, the one or more introns comprises a second intron located on the 3′ end of the first transgenic protein, and wherein the second intron comprises a second RBP-binding hairpin. In some embodiments, a second RBP-binding hairpin is on a nucleic acid sequence located on a 3′ end of the second transgenic protein. In some embodiments, a second RBP-binding hairpin is on a nucleic acid sequence located on the 5′ end of the second transgenic protein. In some embodiments, the one or more introns comprises a first intron located on the 3′ end of the first transgenic protein, and wherein the first intron comprises a first RBP-binding hairpin.

In some embodiments, the one or more expression cassettes comprise a promoter that is expressed during embryogenesis. In some embodiments, the one or more expression cassettes comprises an inducible promoter. In some embodiments, the gene is a non-essential gene. In some embodiments, the gene is an essential gene. In some embodiments, the gene is expressed in an embryo. In some embodiments, the gene is a housekeeping gene that is constitutively expressed. In some embodiments, the non-human vertebrate animal is selected from the group consisting of cow, mouse, rat, rabbit, guinea pig, chicken, fish, bird, reptile, camelid, bovine, chimpanzee, sheep, goat, and non-human primate. In some embodiments, the one or more second sequence variants of the gene do not have sequence identity to the one or more first sequence variant of the gene. In some embodiments, the one or more second sequence variants of the gene have sequence identity to a wildtype sequence of the gene. In some embodiments, the RNA trans-splicing process is spliceosome mediated. In some embodiments, the RNA trans-splicing process is ribozyme mediated. In some embodiments, the Cas polypeptide is a Cas endonuclease. In some embodiments, the Cas endonuclease is a class II Cas endonuclease. In some embodiments, the Cas endonuclease is a type II, type III, or type VI Cas endonuclease. In some embodiments, the Cas endonuclease is an RNA-guided RNA endonuclease. In some embodiments, the Cas endonuclease is selected from the group consisting of Cas9, Cas13, Csm/Cmr, Cas7-11, DisCas7-11, and Cas12a. In some embodiments, the Cas7-11 is Cas7-11a, Cas7-11b, Cas7-11c, or Cas7-11d. In some embodiments, the Cas polypeptide is a variant of the Cas endonuclease. In some embodiments, the Cas polypeptide is an inactive form of the Cas endonuclease. In some embodiments, the Cas polypeptide binds to a polynucleotide but does not cleave the polynucleotide. In some embodiments, the Cas polypeptide is a deactivated Cas13 (dCas13) or a deactivated dDisCas7-11 (dDisCas7-11). In some embodiments, the Cas polypeptide is a dCas13a, dCas13b, dCas13c, or dCas13d. In some embodiments, the Cas polypeptide is a variant of a Prevotella sp. Cas13b (PspCas13b).

In some embodiments, the RBP is selected from the group consisting of MS2 coat protein (MCP), PP7 bacteriophage coat protein, small RNA phage PRR1, and RNA bacteriophages QB coat protein. In some embodiments, the number of the RBP-binding hairpins is at least 1, at least 2, at least 3, at least 4, at least 5, at least 6, at least 7, at least 8, at least 9, or at least 10. In some embodiments, the one or more CRISPR RNA-guided complexes comprise the guide RNA, the Cas polypeptide, the repRNA, or a combination thereof. In some embodiments, the RNA trans-splicing process at a 3′ splicing site results in translation of both the first transgenic protein and the second transgenic protein. In some embodiments, the RNA trans-splicing process at both a 3′ splicing site and a 5′ splicing site results in translation of the first transgenic protein but not the second transgenic protein.

In some embodiments, the gene is an autosomal gene. In some embodiments, the gene is an allosomal gene. In some embodiments, the one or more expression cassettes is located on an autosomal chromosome. In some embodiments, the one or more expression cassettes is integrated into an autosome. In some embodiments, the one or more expression cassettes is located on an allosomal chromosome. In some embodiments, the one or more expression cassettes is integrated into an allosome. In some embodiments, the one or more first sequence variants of the gene comprise a modified intron that is not capable of base pairing with a trans-splicing accepting gene. In some embodiments, the one or more expression cassettes comprising trans-splicing constructs is designed for trans-splicing process with a wildtype nucleic acid sequence of the gene. In some embodiments, the wildtype nucleic acid sequence of the gene comprises one or more second sequence variants of the gene. In some embodiments, the wildtype nucleic acid sequence of the gene further comprises one or more third sequence variants of the gene. In some embodiments, the one or more second sequence variants of the gene do not have sequence identity to the one or more first sequence variant of the gene. In some embodiments, the one or more second sequence variants of the gene do not have sequence identity to the one or more third sequence variant of the gene. In some embodiments, the one or more first sequence variants of the gene do not have sequence identity to the one or more third sequence variant of the gene. In some embodiments, the one or more second sequence variants of the gene and the one or more third sequence variants of the gene do not have sequence identity to each other.

In some embodiments, the trans-splicing accepting gene is a non-essential gene. In some embodiments, the trans-splicing accepting gene is an essential gene. In some embodiments, the trans-splicing accepting gene is expressed in an embryo. In some embodiments, the trans-splicing accepting gene is a housekeeping gene that is constitutively expressed. In some embodiments, the trans-splicing accepting gene is Rictor. In some embodiments, the non-human vertebrate animal is selected from the group consisting of cow, mouse, rat, rabbit, guinea pig, chicken, fish, bird, reptile, camelid, bovine, chimpanzee, sheep, goat, and non-human primate. In some embodiments, the transgenic protein is a toxin. In some embodiments, the toxin is selected from the group consisting of a nuclease, a ribosome toxin, and a protease. In some embodiments, the nuclease comprises Barnase, an RNase, or a restriction endonuclease. In some embodiments, the ribosome toxin comprises diphtheria, ricin, abrin, or pokeweed antiviral protein. In some embodiments, the protease comprises a caspase, proteinase K, trypsin, chymotrypsin, or papain. In some embodiments, the gene is an autosomal gene. In some embodiments, the splice site comprises an acceptor splice site, a donor splice site, or a combination thereof. In some embodiments, the splice site is located at the 5′ end of the transgene. In some embodiments, the splice site is located at the 3′ end of the transgene. In some embodiments, the one or more second sequence variants of the gene do not have sequence identity to the one or more first sequence variant of the gene. In some embodiments, the one or more second sequence variants of the gene have sequence identity to a wildtype sequence of the gene. In some embodiments, the gene is an essential gene, a non-essential gene, a housekeeping gene, or any combination thereof. In some embodiments, the gene is expressed in an embryo. In some embodiments, the one or more expression cassettes further comprise an intron. In some embodiments, when RNA trans-splicing process occurs in the non-human vertebrate animal, the RNA trans-splicing process is spliceosome mediated. In some embodiments, when RNA trans-splicing process occurs in the non-human vertebrate animal, the RNA trans-splicing process is ribozyme mediated. In some embodiments, the Cas polypeptide is a Cas endonuclease. In some embodiments, the Cas endonuclease is a class II Cas endonuclease. In some embodiments, the Cas endonuclease is a type II, type III, or type VI Cas endonuclease. In some embodiments, the Cas endonuclease is an RNA-guided RNA endonuclease. In some embodiments, the Cas endonuclease is Cas9. In some embodiments, the Cas endonuclease is Cas13. In some embodiments, the Cas endonuclease is Csm/Cmr. In some embodiments, the Cas endonuclease is Cas12a. In some embodiments, the Cas endonuclease is Cas7-11. In some embodiments, the Cas7-11 is Cas7-11a, Cas7-11b, Cas7-11c, or Cas7-11d. In some instances, the Cas7-11 is DisCas7-11. In some instances, the DisCas7-11 is dDisCas7-11. In some embodiments, the Cas polypeptide is a variant of the Cas endonuclease. In some embodiments, the Cas polypeptide is an inactive form of the Cas endonuclease. In some embodiments, the Cas polypeptide binds to a polynucleotide but does not cleave the polynucleotide. In some embodiments, the Cas polypeptide is a deactivated Cas13 (dCas13). In some embodiments, the Cas polypeptide is a dCas13a, dCas13b, dCas13c, or dCas13d. In some embodiments, the Cas polypeptide is a variant of a Prevotella sp. Cas13b (PspCas13b). In some embodiments, the number of the RBP-binding hairpins is at least about 1, at least about 2, at least about 3, at least about 4, at least about 5, at least about 6, at least about 7, at least about 8, at least about 9, or at least about 10.

In another aspect, provide herein are a plurality of non-human vertebrate animals comprising: (a) a first non-human vertebrate animal having a genotype comprising: (i) one or more first sequence variants of a gene, wherein the one or more first sequence variants of the gene comprise a modified intron that is not capable of base pairing with a trans-splicing accepting gene; and (ii) one or more expression cassettes comprising: a nucleic acid sequence encoding a replicon RNA (repRNA), comprising a nucleic acid sequence encoding two or more transgenic proteins, two or more splice sites, and one or more introns, wherein the two or more transgenic proteins comprise a first transgenic protein and a second transgenic protein; wherein a 5′ end of the nucleic acid sequence encoding the first transgenic protein comprises a first splice site and a 3′ end of the nucleic acid sequence encoding the first transgenic protein comprises a second splice site; wherein at least one of the one or more introns does not comprise a stop codon; and wherein at least one of the one or more introns comprises a sequence that is complementary to one or more second sequence variants of the gene; and (b) a second non-human vertebrate animal comprising the one or more second variants of the gene. In various aspects, provided herein are a plurality of non-human vertebrate animals comprising: (a) a first non-human vertebrate animal having a genotype comprising: (i) one or more first sequence variants of a gene, wherein the one or more first sequence variants of the gene comprise a modified intron that is not capable of base pairing with a trans-splicing accepting gene; and (ii) one or more expression cassettes comprising: a first nucleic acid sequence encoding a Cas polypeptide linked to an RNA Binding Protein (RBP); a second nucleic acid sequence encoding a replicon RNA (repRNA), comprising a nucleic acid sequence encoding two or more transgenic proteins, two or more splice sites, and one or more introns, wherein the two or more transgenic proteins comprise a first transgenic protein and a second transgenic protein; wherein a 5′ end of the nucleic acid sequence encoding the first transgenic protein comprise a first splice site and a 3′ end of the nucleic acid sequence encoding the first transgenic protein comprises a second splice site; and wherein at least one of the one or more introns (a) is connected to an RBP-binding hairpin, and/or (b) does not comprise a stop codon; and a third nucleic acid sequence encoding a guide RNA (gRNA) capable of directing one or more CRISPR RNA-guided complexes encoded by the one or more expression cassettes to one or more second sequence variants of the gene; and (b) a second non-human vertebrate animal comprising the one or more second variants of the gene. In some embodiments, translation of the first transgenic protein, but not the second transgenic protein, is initiated when RNA trans-splicing occurs at both the first splice site and the second splice site. In some embodiments, translation of each of the first transgenic protein and the second transgenic protein is initiated when RNA trans-splicing occurs only at the first splice site. In some embodiments, the first transgenic protein is located on a 5′ end of the second transgenic protein. In some embodiments, the first splice site is an acceptor splice site. In some embodiments, the second splice site is a donor splice site. In some embodiments, the first transgenic protein is different than the second transgenic protein. In some embodiments, the first transgenic protein is a reporter gene.

In some embodiments, the first transgenic protein is a toxin. In some embodiments, the toxin comprises a nuclease or a protease. In some embodiments, the nuclease comprises a Barnase or an RNase. In some embodiments, the protease comprises a caspase, trypsin, chymotrypsin, or papain. In some embodiments, the second transgenic protein is a cognate toxin inhibitor. In some embodiments, the first transgenic protein is a Barnase or a variant thereof, and the second transgenic protein is a Barstar or a variant thereof. In some embodiments, the first transgenic protein is an RNase, and the second transgenic protein is an RNase inhibitor. In some embodiments, the first transgenic protein is a restriction endonuclease, and the second transgenic protein is In some embodiments, (i) the first transgenic protein is a restriction endonuclease, and the second transgenic protein is a restriction endonuclease inhibitor; (ii) the first transgenic protein is a diphtheria, and the second transgenic protein is a diphtheria antitoxin; (iii) the first transgenic protein is a ricin, and the second transgenic protein is a ricin inhibitor; (iv) the first transgenic protein is an abrin, and the second transgenic protein is an abrin inhibitor; (v) the first transgenic protein is a pokeweed antiviral protein, and the second transgenic protein is a pokeweed antiviral protein inhibitor; (vi) the first transgenic protein is a caspase, and the second transgenic protein is a caspase inhibitor; (vii) the first transgenic protein is a trypsin, and the second transgenic protein is a trypsin inhibitor; (viii) the first transgenic protein is a chymotrypsin, and the second transgenic protein is a chymotrypsin inhibitor; or (ix) the first transgenic protein is a papain, and the second transgenic protein is a papain inhibitor. Other pairs of toxin and cognate toxin inhibitor can be used with the expression cassettes, compositions, or methods described herein.

In other aspects herein, the one or more expression cassettes further comprises a second nucleic acid sequence encoding a Cas polypeptide linked to an RNA Binding Protein (RBP) and a third nucleic acid sequence encoding a guide RNA (gRNA) capable of directing one or more CRISPR RNA-guided complexes encoded by the one or more expression cassettes to one or more second sequence variants of the gene. In some embodiments, the one or more introns comprises a first intron located on the 5′ end of the first transgenic protein, and wherein the first intron comprises a first RBP-binding hairpin. In some embodiments, the one or more introns comprises a second intron located on the 3′ end of the first transgenic protein, and wherein the second intron comprises a second RBP-binding hairpin. In some embodiments, a second RBP-binding hairpin is on a nucleic acid sequence located on a 3′ end of the second transgenic protein. In some embodiments, a second RBP-binding hairpin is on a nucleic acid sequence located on the 5′ end of the second transgenic protein. In some embodiments, the one or more introns comprises a first intron located on the 3′ end of the first transgenic protein, and wherein the first intron comprises a first RBP-binding hairpin.

In some embodiments, the one or more expression cassettes comprise a promoter that is expressed during embryogenesis. In some embodiments, the one or more expression cassettes comprises an inducible promoter. In some embodiments, the gene is a non-essential gene. In some embodiments, the gene is an essential gene. In some embodiments, the gene is expressed in an embryo. In some embodiments, the gene is a housekeeping gene that is constitutively expressed. In some embodiments, the non-human vertebrate animal is selected from the group consisting of cow, mouse, rat, rabbit, guinea pig, chicken, fish, bird, reptile, camelid, bovine, chimpanzee, sheep, goat, and non-human primate. In some embodiments, the one or more second sequence variants of the gene do not have sequence identity to the one or more first sequence variant of the gene. In some embodiments, the one or more second sequence variants of the gene have sequence identity to a wildtype sequence of the gene. In some embodiments, the RNA trans-splicing process is spliceosome mediated. In some embodiments, the RNA trans-splicing process is ribozyme mediated. In some embodiments, the Cas polypeptide is a Cas endonuclease. In some embodiments, the Cas endonuclease is a class II Cas endonuclease. In some embodiments, the Cas endonuclease is a type II, type III, or type VI Cas endonuclease. In some embodiments, the Cas endonuclease is an RNA-guided RNA endonuclease. In some embodiments, the Cas endonuclease is selected from the group consisting of Cas9, Cas13, Csm/Cmr, Cas7-11, DisCas7-11, and Cas12a. In some embodiments, the Cas7-11 is Cas7-11a, Cas7-11b, Cas7-11c, or Cas7-11d. In some embodiments, the Cas polypeptide is a variant of the Cas endonuclease. In some embodiments, the Cas polypeptide is an inactive form of the Cas endonuclease. In some embodiments, the Cas polypeptide binds to a polynucleotide but does not cleave the polynucleotide. In some embodiments, the Cas polypeptide is a deactivated Cas13 (dCas13) or a deactivated dDisCas7-11 (dDisCas7-11). In some embodiments, the Cas polypeptide is a dCas13a, dCas13b, dCas13c, or dCas13d. In some embodiments, the Cas polypeptide is a variant of a Prevotella sp. Cas13b (PspCas13b). In some embodiments, the RBP is selected from the group consisting of MS2 coat protein (MCP), PP7 bacteriophage coat protein, small RNA phage PRR1, and RNA bacteriophages QB coat protein.

In some embodiments, the number of the RBP-binding hairpins is at least 1, at least 2, at least 3, at least 4, at least 5, at least 6, at least 7, at least 8, at least 9, or at least 10. In some embodiments, the one or more CRISPR RNA-guided complexes comprise the guide RNA, the Cas polypeptide, the repRNA, or a combination thereof. In some embodiments, the RNA trans-splicing process at 3′ splicing site results in translation of both the first transgenic protein and the second transgenic protein. In some embodiments, the RNA trans-splicing process at both 3′ splicing site and 5′ splicing site results in translation of the first transgenic protein but not the second transgenic protein. In some embodiments, the gene is an autosomal gene. In some embodiments, the gene is an allosomal gene. In some embodiments, the one or more expression cassettes is located on an autosomal chromosome. In some embodiments, the one or more expression cassettes is integrated into an autosome. In some embodiments, the one or more expression cassettes is located on an allosomal chromosome. In some embodiments, the one or more expression cassettes is integrated into an allosome.

In some embodiments, the gene is an autosomal gene. In some embodiments, the splice site comprises an acceptor splice site, a donor splice site, or a combination thereof. In some embodiments, the splice site is located at the 5′ end of the transgene. In some embodiments, the splice site is located at the 3′ end of the transgene. In some embodiments, the first splice site is a 3′ splice site of the first transgenic protein. In some embodiments, the second splice site is a 5′ splice site of the first transgenic protein. In some embodiments, the one or more second sequence variants of the gene do not have sequence identity to the one or more first sequence variant of the gene. In some embodiments, the one or more second sequence variants of the gene have sequence identity to a wildtype sequence of the gene. In some embodiments, the gene is an essential gene, a non-essential gene, a housekeeping gene, or any combination thereof. In some embodiments, the gene is expressed in an embryo. In some embodiments, the gene is Rictor. In some embodiments, the one or more expression cassettes further comprise an intron. In some embodiments, when RNA trans-splicing process occurs in the non-human vertebrate animal, the RNA trans-splicing process is spliceosome mediated. In some embodiments, when the RNA trans-splicing process occurs in the non-human vertebrate animal, the RNA trans-splicing process is ribozyme mediated. In some embodiments, the Cas polypeptide is a Cas endonuclease. In some embodiments, the Cas endonuclease is a class II Cas endonuclease. In some embodiments, the Cas endonuclease is a type II, type III, or type VI Cas endonuclease. In some embodiments, the Cas endonuclease is an RNA-guided RNA endonuclease. In some embodiments, the Cas endonuclease is Cas9. In some embodiments, the Cas endonuclease is Cas13. In some embodiments, the Cas endonuclease is Csm/Cmr. In some embodiments, the Cas endonuclease is Cas12a. In some embodiments, the Cas endonuclease is Cas7-11. In some embodiments, the Cas7-11 is Cas7-11a, Cas7-11b, Cas7-11c, or Cas7-11d. In some instances, the Cas7-11 is DisCas7-11. In some instances, the DisCas7-11 is dDisCas7-11. In some embodiments, the Cas polypeptide is a variant of the Cas endonuclease. In some embodiments, the Cas polypeptide is an inactive form of the Cas endonuclease. In some embodiments, the Cas polypeptide binds to a polynucleotide but does not cleave the polynucleotide. In some embodiments, the Cas polypeptide is a deactivated Cas13 (dCas13). In some embodiments, the Cas polypeptide is a dCas13a, dCas13b, dCas13c, or dCas13d. In some embodiments, the Cas polypeptide is a variant of a Prevotella sp. Cas13b (PspCas13b). In some embodiments, the number of the RBP-binding hairpins is at least about 1, at least about 2, at least about 3, at least about 4, at least about 5, at least about 6, at least about 7, at least about 8, at least about 9, or at least about 10.

In another aspect, as described herein, an engineered animal in a parental generation can be generated to harbor one or more expression cassette(s) for an RNA trans-splicing process and one or more first sequence variants of the gene comprising a modified intron that is not capable of the RNA trans-splicing process. In some instances, the one or more expression cassette(s) for the RNA trans-splicing process can be located on the same chromosome as the one or more first sequence variants of the gene comprising a modified intron that is not capable of the trans-splicing process. In another instance, the one or more expression cassette(s) for the RNA trans-splicing process can be located on a different chromosome than the one or more first sequence variants of the gene comprising a modified intron that is not capable of the trans-splicing process. Further, in another aspect, provided herein are exemplary embodiments of an engineered animal in a parental generation useful for generating a single sex population of non-human vertebrate animals.

### Poultry

In one aspect, the present disclosure provides an engineered poultry for generation of single sex offspring, e.g., female layer hens. In some instances, a female chicken in the parental generation is engineered to harbor one or more expression cassette(s) for the RNA trans-splicing process, wherein the one or more expression cassette(s) comprises a nucleic acid sequence encoding a replicon RNA (repRNA), comprising a nucleic acid sequence encoding two or more transgenic proteins, two or more splice sites, and one or more introns, wherein the two or more transgenic proteins comprise a first transgenic protein and a second transgenic protein; wherein a 5′ end of the nucleic acid sequence encoding the first transgenic protein comprises a first splice site and a 3′ end of the nucleic acid sequence encoding the first transgenic protein comprises a second splice site; wherein at least one of the one or more introns does not comprise a stop codon; and wherein at least one of the one or more introns comprises a sequence that is complementary to one or more second sequence variants of the gene. In some embodiments, a female chicken in the parental generation is engineered to harbor one or more expression cassette(s) for the RNA trans-splicing process, wherein the one or more expression cassette(s) comprises a nucleic acid sequence encoding a replicon RNA (repRNA), comprising a nucleic acid sequence encoding two or more transgenic proteins, two or more splice sites, and one or more introns, wherein the two or more transgenic proteins comprise a first transgenic protein and a second transgenic protein; wherein a 5′ end of the nucleic acid sequence encoding the first transgenic protein comprises a first splice site and a 3′ end of the nucleic acid sequence encoding the first transgenic protein comprises a second splice site; wherein the first transgenic protein is located on a 5′ end of the second transgenic protein; wherein at least one of the one or more introns does not comprise a stop codon; and wherein at least one of the one or more introns comprises a sequence that is complementary to one or more second sequence variants of the gene. In some embodiments, a female chicken in the parental generation is engineered to harbor one or more expression cassette(s) for the RNA trans-splicing process, wherein the one or more expression cassette(s) comprises (i) a first nucleic acid sequence encoding a Cas polypeptide linked to an RNA Binding Protein (RBP); (ii) a second nucleic acid sequence encoding a replicon RNA (repRNA), comprising a nucleic acid sequence encoding two or more transgenic proteins, two or more splice sites, and one or more introns, wherein the two or more transgenic proteins comprise a first transgenic protein and a second transgenic protein; wherein a 5′ end of the nucleic acid sequence encoding the first transgenic protein comprises a first splice site and a 3′ end of the nucleic acid sequence encoding the first transgenic protein comprises a second splice site; and wherein at least one of the one or more introns (a) is connected to an RBP-binding hairpin, and/or (b) does not comprise a stop codon; and (iii) a third nucleic acid sequence encoding a guide RNA (gRNA) capable of directing one or more CRISPR RNA-guided complexes encoded by the one or more expression cassettes to one or more second sequence variants of the gene. In some embodiments, a female chicken in the parental generation is engineered to harbor one or more expression cassette(s) for the RNA trans-splicing process, wherein the one or more expression cassette(s) comprises (i) a first nucleic acid sequence encoding a Cas polypeptide linked to an RNA Binding Protein (RBP); (ii) a second nucleic acid sequence encoding a replicon RNA (repRNA), comprising a nucleic acid sequence encoding two or more transgenic proteins, two or more splice sites, and one or more introns, wherein the two or more transgenic proteins comprise a first transgenic protein and a second transgenic protein; wherein a 5′ end of the nucleic acid sequence encoding the first transgenic protein comprises a first splice site and a 3′ end of the nucleic acid sequence encoding the first transgenic protein comprises a second splice site; and wherein at least one of the one or more introns (a) is connected to an RBP-binding hairpin, and/or (b) does not comprise a stop codon; wherein at least one of the one or more introns comprises a sequence that is complementary to one or more second sequence variants of the gene; and (iii) a third nucleic acid sequence encoding a guide RNA (gRNA) capable of directing one or more CRISPR RNA-guided complexes encoded by the one or more expression cassettes to one or more third sequence variants of the gene, wherein the one or more second sequence variants of the gene and the one or more third sequence variants of the gene do not have sequence identity to each other.

In some instances, the one or more expression cassettes herein is integrated into the female chicken chromosome. In some instances, the one or more expression cassette(s) is integrated into the Z allosome (called Z¹ or). In this instance, the genotype of the engineered female chicken is Z¹W or W. In this instance, the female chicken is further engineered to harbor one or more first sequence variants of a gene, wherein the one or more first sequence variants of the gene comprise one or more nucleotide sequences comprising a modified intron that is not capable of the trans-splicing process with a target pre-mRNA. In some instances, the female chicken is further engineered to harbor one or more first sequence variants of a gene, wherein the one or more first sequence variants of the gene comprise one or more nucleotide sequences comprising a modified intron that is not capable of base pairing with a trans-splicing accepting gene. In another embodiment, the female chicken is further engineered to harbor one or more first sequence variants of a gene and the one or more expression cassettes on the allosome. In another embodiment, the female chicken is further engineered to harbor one or more first sequence variants of an allosomal gene and the one or more expression cassettes on the allosome. The genotype of this engineered female chicken is Z¹W or W. In these engineered female chickens, the RNA trans-splicing process cannot occur. These engineered female chickens can be bred with any lines of wildtype male chicken to generate single sex offspring, e.g., female layer hens.

The genotype of wildtype male chicken is ZZ, thus, when crossing with the engineered female chicken Z¹W or W, male offspring will have the genotype of Z¹Z or Z, while female offspring will have genotype ZW. Because Z¹ or carries one or more expression cassette(s) for RNA trans-splicing process, in some embodiments, if the RNA trans-splicing occurs at both 3′ splicing site and 5′ splicing site, translation of the first transgenic protein but not the second transgenic protein occurs, resulting in a non-viable male offspring. In some embodiments, translation of a first transgenic protein of the two or more transgenic proteins is initiated when RNA trans-splicing occurs at both the first splice site and the second splice site. In some embodiments, male offspring expressing the first transgenic protein and not the second transgenic protein are visually identifiable, e.g., the transgenic protein comprises a fluorescent protein such as one or more of a green fluorescent protein (GFP), yellow fluorescent protein (YFP), red fluorescent protein (RFP), blue fluorescent protein (BFP), cyan fluorescent protein (CFP), and orange fluorescent protein (OFP). Thus, generation of single sex offspring, e.g., female chicken, is achieved via enhanced RNA trans-splicing process to express the first transgenic protein, e.g., toxin.

In some embodiments, the engineered female poultry comprises a modified allosome (Z chromosome) comprising one or more first sequence variants of a gene, wherein the one or more first sequence variants of the gene comprise a modified intron that is not capable of base pairing with a trans-splicing accepting gene; and one or more expression cassettes comprising a nucleic acid sequence encoding a replicon RNA (repRNA), comprising a nucleic acid sequence encoding two or more transgenic proteins, two or more splice sites, and one or more introns, wherein the two or more transgenic proteins comprise a first transgenic protein and a second transgenic protein; wherein a 5′ end of the nucleic acid sequence encoding the first transgenic protein comprises a first splice site and a 3′ end of the nucleic acid sequence encoding the first transgenic protein comprises a second splice site; wherein at least one of the one or more introns does not comprise a stop codon; and wherein at least one of the one or more introns comprises a sequence that is complementary to one or more second sequence variants of the gene. In some embodiments, translation of the first transgenic protein, but not the second transgenic protein, is initiated when RNA trans-splicing occurs at both the first splice site and the second splice site. In some embodiments, translation of each of the first transgenic protein and the second transgenic protein is initiated when RNA trans-splicing occurs only at the first splice site. In some embodiments, the first transgenic protein is located on a 5′ end of the second transgenic protein. In some embodiments, the first splice site is an acceptor splice site. In some embodiments, the second splice site is a donor splice site. In some embodiments, the first transgenic protein is different than the second transgenic protein. In some embodiments, the first transgenic protein is a reporter gene. In some embodiments, the first transgenic protein is a toxin. In some embodiments, the second transgenic protein is a cognate toxin inhibitor. In some embodiments, the one or more expression cassettes further comprises a second nucleic acid sequence encoding a Cas polypeptide linked to an RNA Binding Protein (RBP) and a third nucleic acid sequence encoding a guide RNA (gRNA) capable of directing one or more CRISPR RNA-guided complexes encoded by the one or more expression cassettes to one or more second sequence variants of the gene. In some embodiments, the one or more introns comprises a first intron located on the 5′ end of the first transgenic protein, and wherein the first intron comprises a first RBP-binding hairpin. In some embodiments, the one or more introns comprises a second intron located on the 3′ end of the first transgenic protein, and wherein the second intron comprises a second RBP-binding hairpin. In some embodiments, a second RBP-binding hairpin is on a nucleic acid sequence located on a 3′ end of the second transgenic protein. In some embodiments, a second RBP-binding hairpin is on a nucleic acid sequence located on the 5′ end of the second transgenic protein. In some embodiments, the one or more introns comprises a first intron located on the 3′ end of the first transgenic protein, and wherein the first intron comprises a first RBP-binding hairpin. In some embodiments, the engineered female poultry comprises a modified allosome (Z chromosome) comprising one or more first sequence variants of a gene, wherein the one or more first sequence variants of the gene comprise a modified intron that is not capable of base pairing with a trans-splicing accepting gene; and (I) one or more expression cassettes comprising: a nucleic acid sequence encoding a replicon RNA (repRNA), comprising a nucleic acid sequence encoding two or more transgenic proteins, two or more splice sites, and one or more introns, wherein the two or more transgenic proteins comprise a first transgenic protein and a second transgenic protein; wherein a 5′ end of the nucleic acid sequence encoding the first transgenic protein comprises a first splice site and a 3′ end of the nucleic acid sequence encoding the first transgenic protein comprises a second splice site; wherein the first transgenic protein is located on a 5′ end of the second transgenic protein; wherein at least one of the one or more introns does not comprise a stop codon; and wherein at least one of the one or more introns comprises a sequence that is complementary to one or more second sequence variants of the gene; (II) one or more expression cassettes comprising: (i) a first nucleic acid sequence encoding a Cas polypeptide linked to an RNA Binding Protein (RBP); (ii) a second nucleic acid sequence encoding a replicon RNA (repRNA), comprising a nucleic acid sequence encoding two or more transgenic proteins, two or more splice sites, and one or more introns, wherein the two or more transgenic proteins comprise a first transgenic protein and a second transgenic protein; wherein a 5′ end of the nucleic acid sequence encoding the first transgenic protein comprises a first splice site and a 3′ end of the nucleic acid sequence encoding the first transgenic protein comprises a second splice site; and wherein at least one of the one or more introns (a) is connected to an RBP-binding hairpin, and/or (b) does not comprise a stop codon; and (iii) a third nucleic acid sequence encoding a guide RNA (gRNA) capable of directing one or more CRISPR RNA-guided complexes encoded by the one or more expression cassettes to one or more second sequence variants of the gene; or (III) one or more expression cassettes comprises (i) a first nucleic acid sequence encoding a Cas polypeptide linked to an RNA Binding Protein (RBP); (ii) a second nucleic acid sequence encoding a replicon RNA (repRNA), comprising a nucleic acid sequence encoding two or more transgenic proteins, two or more splice sites, and one or more introns, wherein the two or more transgenic proteins comprise a first transgenic protein and a second transgenic protein; wherein a 5′ end of the nucleic acid sequence encoding the first transgenic protein comprises a first splice site and a 3′ end of the nucleic acid sequence encoding the first transgenic protein comprises a second splice site; and wherein at least one of the one or more introns (a) is connected to an RBP-binding hairpin, and/or (b) does not comprise a stop codon; wherein at least one of the one or more introns comprises a sequence that is complementary to one or more second sequence variants of the gene; and (iii) a third nucleic acid sequence encoding a guide RNA (gRNA) capable of directing one or more CRISPR RNA-guided complexes encoded by the one or more expression cassettes to one or more third sequence variants of the gene, wherein the one or more second sequence variants of the gene and the one or more third sequence variants of the gene do not have sequence identity to each other. In these engineered female chickens, the RNA trans-splicing process cannot occur. These engineered female chickens can be bred with any lines of wildtype male chicken to generate single sex offspring, e.g., female layer hens.

The genotype of wildtype male chicken is ZZ, thus, when crossing with the engineered female chicken W, male offspring will have the genotype of Z, while female offspring will have genotype ZW. Because carries one or more expression cassette(s) for RNA trans-splicing process, in some embodiments, if the RNA trans-splicing occurs at both 3′ splicing site and 5′ splicing site, translation of the first transgenic protein, e.g., a toxin, but not the second transgenic protein occurs, resulting in a non-viable male offspring. In some embodiments, translation of the first transgenic protein and the second transgenic protein is initiated when RNA trans-splicing occurs only at the first splice site. In some embodiments, translation of the first transgenic protein, but not the second transgenic protein, is initiated when RNA trans-splicing occurs at both the first splice site and the second splice site. This RNA trans-splicing system for generation of single sex offspring can be applied to other animals, all of which are compatible with methods of the present disclosure and contemplated herein. Examples of animals include, but is not limited to chicken, bird, and reptile.

In another aspect, the present disclosure provides an engineered poultry for generation of single sex offspring, e.g., a female chicken. In some instances, a female chicken in the parental generation is engineered to harbor one or more expression cassette(s) for RNA trans-splicing process, wherein one or more expression cassettes comprising a nucleic acid sequence encoding a replicon RNA (repRNA), comprising a nucleic acid sequence encoding two or more transgenic proteins, two or more splice sites, and one or more introns, wherein the two or more transgenic proteins comprise a first transgenic protein and a second transgenic protein; wherein a 5′ end of the nucleic acid sequence encoding the first transgenic protein comprises a first splice site and a 3′ end of the nucleic acid sequence encoding the first transgenic protein comprises a second splice site; wherein at least one of the one or more introns does not comprise a stop codon; and wherein at least one of the one or more introns comprises a sequence that is complementary to one or more second sequence variants of the gene. In some embodiments, translation of the first transgenic protein, but not the second transgenic protein, is initiated when RNA trans-splicing occurs at both the first splice site and the second splice site. In some embodiments, translation of each of the first transgenic protein and the second transgenic protein is initiated when RNA trans-splicing occurs only at the first splice site. In some embodiments, the first transgenic protein is located on a 5′ end of the second transgenic protein. In some embodiments, the first splice site is an acceptor splice site. In some embodiments, the second splice site is a donor splice site. In some embodiments, the first transgenic protein is different than the second transgenic protein. In some embodiments, the first transgenic protein is a reporter gene. In some embodiments, the first transgenic protein is a toxin. In some embodiments, the second transgenic protein is a cognate toxin inhibitor. In some embodiments, the one or more expression cassettes further comprises a second nucleic acid sequence encoding a Cas polypeptide linked to an RNA Binding Protein (RBP) and a third nucleic acid sequence encoding a guide RNA (gRNA) capable of directing one or more CRISPR RNA-guided complexes encoded by the one or more expression cassettes to one or more second sequence variants of the gene. In some embodiments, the one or more introns comprises a first intron located on the 5′ end of the first transgenic protein, and wherein the first intron comprises a first RBP-binding hairpin. In some embodiments, the one or more introns comprises a second intron located on the 3′ end of the first transgenic protein, and wherein the second intron comprises a second RBP-binding hairpin. In some embodiments, a second RBP-binding hairpin is on a nucleic acid sequence located on a 3′ end of the second transgenic protein. In some embodiments, a second RBP-binding hairpin is on a nucleic acid sequence located on the 5′ end of the second transgenic protein. In some embodiments, the one or more introns comprises a first intron located on the 3′ end of the first transgenic protein, and wherein the first intron comprises a first RBP-binding hairpin. In some embodiments, a female chicken in the parental generation is engineered to harbor (I) one or more expression cassettes comprising: a nucleic acid sequence encoding a replicon RNA (repRNA), comprising a nucleic acid sequence encoding two or more transgenic proteins, two or more splice sites, and one or more introns, wherein the two or more transgenic proteins comprise a first transgenic protein and a second transgenic protein; wherein a 5′ end of the nucleic acid sequence encoding the first transgenic protein comprises a first splice site and a 3′ end of the nucleic acid sequence encoding the first transgenic protein comprises a second splice site; wherein the first transgenic protein is located on a 5′ end of the second transgenic protein; wherein at least one of the one or more introns does not comprise a stop codon; and wherein at least one of the one or more introns comprises a sequence that is complementary to one or more second sequence variants of the gene; (II) one or more expression cassettes comprising: (i) a first nucleic acid sequence encoding a Cas polypeptide linked to an RNA Binding Protein (RBP); (ii) a second nucleic acid sequence encoding a replicon RNA (repRNA), comprising a nucleic acid sequence encoding two or more transgenic proteins, two or more splice sites, and one or more introns, wherein the two or more transgenic proteins comprise a first transgenic protein and a second transgenic protein; wherein a 5′ end of the nucleic acid sequence encoding the first transgenic protein comprises a first splice site and a 3′ end of the nucleic acid sequence encoding the first transgenic protein comprises a second splice site; and wherein at least one of the one or more introns (a) is connected to an RBP-binding hairpin, and/or (b) does not comprise a stop codon; and (iii) a third nucleic acid sequence encoding a guide RNA (gRNA) capable of directing one or more CRISPR RNA-guided complexes encoded by the one or more expression cassettes to one or more second sequence variants of the gene; or (III) one or more expression cassettes comprises (i) a first nucleic acid sequence encoding a Cas polypeptide linked to an RNA Binding Protein (RBP); (ii) a second nucleic acid sequence encoding a replicon RNA (repRNA), comprising a nucleic acid sequence encoding two or more transgenic proteins, two or more splice sites, and one or more introns, wherein the two or more transgenic proteins comprise a first transgenic protein and a second transgenic protein; wherein a 5′ end of the nucleic acid sequence encoding the first transgenic protein comprises a first splice site and a 3′ end of the nucleic acid sequence encoding the first transgenic protein comprises a second splice site; and wherein at least one of the one or more introns (a) is connected to an RBP-binding hairpin, and/or (b) does not comprise a stop codon; wherein at least one of the one or more introns comprises a sequence that is complementary to one or more second sequence variants of the gene; and (iii) a third nucleic acid sequence encoding a guide RNA (gRNA) capable of directing one or more CRISPR RNA-guided complexes encoded by the one or more expression cassettes to one or more third sequence variants of the gene, wherein the one or more second sequence variants of the gene and the one or more third sequence variants of the gene do not have sequence identity to each other. This expression cassette is integrated into the female chicken chromosome. In some instances, the one or more expression cassette(s) is integrated into the Z allosome (called Z¹ or Z). In this instance, the genotype of the engineered female chicken is Z¹W. Further, in this instance, the female chicken is engineered to harbor one or more first sequence variants of a gene, indicated as A*, wherein the one or more first sequence variants of the gene comprise one or more nucleotide sequences comprising a modified intron that is not capable of base pairing with a trans-splicing accepting gene. In some instances, both alleles of the gene are modified, and the genotype of this engineered female chicken is A*A* and Z¹W. In this engineered female chicken, the RNA trans-splicing process cannot occur. This engineered female chicken can be bred with any lines of wildtype male chicken to generate single sex offspring, e.g., a male chicken.

The genotype of wildtype male chicken is AA and ZZ, thus, when crossing with the engineered female chicken A*A* and Z¹W, male offspring have the genotype of A*A and Z¹Z and female offspring have the genotype of A*A and ZW. In some embodiments, translation of the first transgenic proteins, but not the second transgenic protein, is initiated when RNA trans-splicing occurs at both the first splice site and the second splice site. Because Z¹ carries one or more expression(s) cassette for RNA trans-splicing process with wildtype sequence in the “A” chromosome to express the first transgenic protein, e.g., a toxin, male offspring express the toxin and are not viable. In some cases, expression of the transgenic protein by the resulting progeny makes them visually identifiable, e.g., when the first transgenic protein is a fluorescent protein. In some embodiments, the transgenic protein comprises one or more of a green fluorescent protein (GFP), yellow fluorescent protein (YFP), red fluorescent protein (RFP), blue fluorescent protein (BFP), cyan fluorescent protein (CFP), and orange fluorescent protein (OFP). Thus, generation of single sex offspring, e.g., a male chicken, is achieved via enhanced RNA trans-splicing process to express the transgenic protein, e.g., a toxin.

In another aspect, the present disclosure provides an engineered poultry for generation of single sex offspring, e.g., a male chicken. In some instances, a female chicken in the parental generation is engineered to harbor one or more expression cassette(s) for RNA trans-splicing process, wherein one or more expression cassettes comprising a nucleic acid sequence encoding a replicon RNA (repRNA), comprising a nucleic acid sequence encoding two or more transgenic proteins, two or more splice sites, and one or more introns, wherein the two or more transgenic proteins comprise a first transgenic protein and a second transgenic protein; wherein a 5′ end of the nucleic acid sequence encoding the first transgenic protein comprises a first splice site and a 3′ end of the nucleic acid sequence encoding the first transgenic protein comprises a second splice site; wherein at least one of the one or more introns does not comprise a stop codon; and wherein at least one of the one or more introns comprises a sequence that is complementary to one or more second sequence variants of the gene. In some embodiments, translation of the first transgenic protein, but not the second transgenic protein, is initiated when RNA trans-splicing occurs at both the first splice site and the second splice site. In some embodiments, translation of each of the first transgenic protein and the second transgenic protein is initiated when RNA trans-splicing occurs only at the first splice site. In some embodiments, the first transgenic protein is located on a 5′ end of the second transgenic protein. In some embodiments, the first splice site is an acceptor splice site. In some embodiments, the second splice site is a donor splice site. In some embodiments, the first transgenic protein is different than the second transgenic protein. In some embodiments, the first transgenic protein is a reporter gene. In some embodiments, the first transgenic protein is a toxin. In some embodiments, the second transgenic protein is a cognate toxin inhibitor. In some embodiments, the one or more expression cassettes further comprises a second nucleic acid sequence encoding a Cas polypeptide linked to an RNA Binding Protein (RBP) and a third nucleic acid sequence encoding a guide RNA (gRNA) capable of directing one or more CRISPR RNA-guided complexes encoded by the one or more expression cassettes to one or more second sequence variants of the gene. In some embodiments, the one or more introns comprises a first intron located on the 5′ end of the first transgenic protein, and wherein the first intron comprises a first RBP-binding hairpin. In some embodiments, the one or more introns comprises a second intron located on the 3′ end of the first transgenic protein, and wherein the second intron comprises a second RBP-binding hairpin. In some embodiments, a second RBP-binding hairpin is on a nucleic acid sequence located on a 3′ end of the second transgenic protein. In some embodiments, a second RBP-binding hairpin is on a nucleic acid sequence located on the 5′ end of the second transgenic protein. In some embodiments, the one or more introns comprises a first intron located on the 3′ end of the first transgenic protein, and wherein the first intron comprises a first RBP-binding hairpin. In some instances, a female chicken in the parental generation is engineered to harbor (I) one or more expression cassettes comprising: a nucleic acid sequence encoding a replicon RNA (repRNA), comprising a nucleic acid sequence encoding two or more transgenic proteins, two or more splice sites, and one or more introns, wherein the two or more transgenic proteins comprise a first transgenic protein and a second transgenic protein; wherein a 5′ end of the nucleic acid sequence encoding the first transgenic protein comprises a first splice site and a 3′ end of the nucleic acid sequence encoding the first transgenic protein comprises a second splice site; wherein the first transgenic protein is located on a 5′ end of the second transgenic protein; wherein at least one of the one or more introns does not comprise a stop codon; and wherein at least one of the one or more introns comprises a sequence that is complementary to one or more second sequence variants of the gene; (II) one or more expression cassettes comprising: (i) a first nucleic acid sequence encoding a Cas polypeptide linked to an RNA Binding Protein (RBP); (ii) a second nucleic acid sequence encoding a replicon RNA (repRNA), comprising a nucleic acid sequence encoding two or more transgenic proteins, two or more splice sites, and one or more introns, wherein the two or more transgenic proteins comprise a first transgenic protein and a second transgenic protein; wherein a 5′ end of the nucleic acid sequence encoding the first transgenic protein comprises a first splice site and a 3′ end of the nucleic acid sequence encoding the first transgenic protein comprises a second splice site; and wherein at least one of the one or more introns (a) is connected to an RBP-binding hairpin, and/or (b) does not comprise a stop codon; and (iii) a third nucleic acid sequence encoding a guide RNA (gRNA) capable of directing one or more CRISPR RNA-guided complexes encoded by the one or more expression cassettes to one or more second sequence variants of the gene; or (III) one or more expression cassettes comprises (i) a first nucleic acid sequence encoding a Cas polypeptide linked to an RNA Binding Protein (RBP); (ii) a second nucleic acid sequence encoding a replicon RNA (repRNA), comprising a nucleic acid sequence encoding two or more transgenic proteins, two or more splice sites, and one or more introns, wherein the two or more transgenic proteins comprise a first transgenic protein and a second transgenic protein; wherein a 5′ end of the nucleic acid sequence encoding the first transgenic protein comprises a first splice site and a 3′ end of the nucleic acid sequence encoding the first transgenic protein comprises a second splice site; and wherein at least one of the one or more introns (a) is connected to an RBP-binding hairpin, and/or (b) does not comprise a stop codon; wherein at least one of the one or more introns comprises a sequence that is complementary to one or more second sequence variants of the gene; and (iii) a third nucleic acid sequence encoding a guide RNA (gRNA) capable of directing one or more CRISPR RNA-guided complexes encoded by the one or more expression cassettes to one or more third sequence variants of the gene, wherein the one or more second sequence variants of the gene and the one or more third sequence variants of the gene do not have sequence identity to each other. This expression cassette is integrated into the female chicken chromosome. In some instances, the one or more expression cassette(s) is integrated into the W allosome (called W¹ or). In this instance, the genotype of the engineered female chicken is ZW¹. Further, in this instance, the female chicken is engineered to harbor one or more first sequence variants of a gene, indicated as A*, wherein the one or more first sequence variants of the gene comprise one or more nucleotide sequences comprising a modified intron that is not capable of base pairing with a trans-splicing accepting gene. In some instances, both alleles of the gene are modified, and the genotype of this engineered female chicken is A*A* and ZW¹. In this engineered female chicken, the RNA trans-splicing process cannot occur. This engineered female chicken can be bred with any lines of wildtype male chicken to generate single sex offspring, e.g., a male chicken.

The genotype of wildtype male chicken is AA and ZZ, thus, when crossing with the engineered female chicken A*A* and ZW¹, male offspring have the genotype of A*A and ZZ, and female offspring have the genotype of A*A and ZW¹. In some instances, the expression of the first transgenic protein occurs when the RNA trans-splicing process occurs at both the first splice site and the second splice site. In some embodiments, translation of the first transgenic proteins, but not the second transgenic protein, is initiated when RNA trans-splicing occurs at both the first splice site and the second splice site. Because W¹ carries one or more expression(s) cassette for RNA trans-splicing process with wildtype sequence in the “A” chromosome to express the first transgenic protein, e.g., a toxin, female offspring express toxin protein and not viable. In some cases, expression of the transgenic protein by the resulting progeny makes them visually identifiable, e.g., when the first transgenic protein is a fluorescent protein. In some embodiments, the transgenic protein comprises one or more of a green fluorescent protein (GFP), yellow fluorescent protein (YFP), red fluorescent protein (RFP), blue fluorescent protein (BFP), cyan fluorescent protein (CFP), and orange fluorescent protein (OFP). Thus, generation of single sex offspring, e.g., a male chicken, is achieved via enhanced RNA trans-splicing process to express the transgenic protein, e.g., a toxin.

In some embodiments, translation of the first transgenic protein and the second transgenic protein is initiated when RNA trans-splicing occurs only at the first splice site. In some embodiments, translation of the first transgenic protein, but not the second transgenic protein, is initiated when RNA trans-splicing occurs at both the first splice site and the second splice site. This RNA trans-splicing system for generation of single sex offspring can be applied to other animals, all of which are compatible with methods of the present disclosure and contemplated herein. Examples of animal include, but are not limited to chicken, bird, and reptile.

### Mammals

In one aspect, the present disclosure provides an engineered mammal, e.g., a cow, for generation of single sex offspring, e.g., a female cow. In some instances, a male cow in the parental generation is engineered to harbor one or more expression cassette(s) for RNA trans-splicing process, wherein one or more expression cassettes comprising a nucleic acid sequence encoding a replicon RNA (repRNA), comprising a nucleic acid sequence encoding two or more transgenic proteins, two or more splice sites, and one or more introns, wherein the two or more transgenic proteins comprise a first transgenic protein and a second transgenic protein; wherein a 5′ end of the nucleic acid sequence encoding the first transgenic protein comprises a first splice site and a 3′ end of the nucleic acid sequence encoding the first transgenic protein comprises a second splice site; wherein at least one of the one or more introns does not comprise a stop codon; and wherein at least one of the one or more introns comprises a sequence that is complementary to one or more second sequence variants of the gene. In some embodiments, translation of the first transgenic protein, but not the second transgenic protein, is initiated when RNA trans-splicing occurs at both the first splice site and the second splice site. In some embodiments, translation of each of the first transgenic protein and the second transgenic protein is initiated when RNA trans-splicing occurs only at the first splice site. In some embodiments, the first transgenic protein is located on a 5′ end of the second transgenic protein. In some embodiments, the first splice site is an acceptor splice site. In some embodiments, the second splice site is a donor splice site. In some embodiments, the first transgenic protein is different than the second transgenic protein. In some embodiments, the first transgenic protein is a reporter gene. In some embodiments, the first transgenic protein is a toxin. In some embodiments, the second transgenic protein is a cognate toxin inhibitor. In some embodiments, the one or more expression cassettes further comprises a second nucleic acid sequence encoding a Cas polypeptide linked to an RNA Binding Protein (RBP) and a third nucleic acid sequence encoding a guide RNA (gRNA) capable of directing one or more CRISPR RNA-guided complexes encoded by the one or more expression cassettes to one or more second sequence variants of the gene. In some embodiments, the one or more introns comprises a first intron located on the 5′ end of the first transgenic protein, and wherein the first intron comprises a first RBP-binding hairpin. In some embodiments, the one or more introns comprises a second intron located on the 3′ end of the first transgenic protein, and wherein the second intron comprises a second RBP-binding hairpin. In some embodiments, a second RBP-binding hairpin is on a nucleic acid sequence located on a 3′ end of the second transgenic protein. In some embodiments, a second RBP-binding hairpin is on a nucleic acid sequence located on the 5′ end of the second transgenic protein. In some embodiments, the one or more introns comprises a first intron located on the 3′ end of the first transgenic protein, and wherein the first intron comprises a first RBP-binding hairpin. In some instances, a male cow in the parental generation is engineered to harbor (I) one or more expression cassettes comprising: a nucleic acid sequence encoding a replicon RNA (repRNA), comprising a nucleic acid sequence encoding two or more transgenic proteins, two or more splice sites, and one or more introns, wherein the two or more transgenic proteins comprise a first transgenic protein and a second transgenic protein; wherein a 5′ end of the nucleic acid sequence encoding the first transgenic protein comprises a first splice site and a 3′ end of the nucleic acid sequence encoding the first transgenic protein comprises a second splice site; wherein the first transgenic protein is located on a 5′ end of the second transgenic protein; wherein at least one of the one or more introns does not comprise a stop codon; and wherein at least one of the one or more introns comprises a sequence that is complementary to one or more second sequence variants of the gene; (II) one or more expression cassettes comprising: (i) a first nucleic acid sequence encoding a Cas polypeptide linked to an RNA Binding Protein (RBP); (ii) a second encoding two or more transgenic proteins, two or more splice sites, and one or more introns, wherein the two or more transgenic proteins comprise a first transgenic protein and a second transgenic protein; wherein a 5′ end of the nucleic acid sequence encoding the first transgenic protein comprises a first splice site and a 3′ end of the nucleic acid sequence encoding the first transgenic protein comprises a second splice site; and wherein at least one of the one or more introns (a) is connected to an RBP-binding hairpin, and/or (b) does not comprise a stop codon; and (iii) a third nucleic acid sequence encoding a guide RNA (gRNA) capable of directing one or more CRISPR RNA-guided complexes encoded by the one or more expression cassettes to one or more second sequence variants of the gene; or (III) one or more expression cassettes comprises (i) a first nucleic acid sequence encoding a Cas polypeptide linked to an RNA Binding Protein (RBP); (ii) a second nucleic acid sequence encoding a replicon RNA (repRNA), comprising a nucleic acid sequence encoding two or more transgenic proteins, two or more splice sites, and one or more introns, wherein the two or more transgenic proteins comprise a first transgenic protein and a second transgenic protein; wherein a 5′ end of the nucleic acid sequence encoding the first transgenic protein comprises a first splice site and a 3′ end of the nucleic acid sequence encoding the first transgenic protein comprises a second splice site; and wherein at least one of the one or more introns (a) is connected to an RBP-binding hairpin, and/or (b) does not comprise a stop codon; wherein at least one of the one or more introns comprises a sequence that is complementary to one or more second sequence variants of the gene; and (iii) a third nucleic acid sequence encoding a guide RNA (gRNA) capable of directing one or more CRISPR RNA-guided complexes encoded by the one or more expression cassettes to one or more third sequence variants of the gene, wherein the one or more second sequence variants of the gene and the one or more third sequence variants of the gene do not have sequence identity to each other. This expression cassette is integrated into the male cow chromosome. In some instances, the one or more expression cassette(s) is integrated into the Y allosome (called Y¹ or). In this instance, the genotype of the engineered male cow is XY¹. Further, in this instance, the male cow is engineered to harbor one or more first sequence variants of a gene, indicated as A*, wherein the one or more first sequence variants of the gene comprise one or more nucleotide sequences comprising a modified intron that is not capable of base pairing with a trans-splicing accepting gene. In some instances, both allele of the gene is modified, and the genotype of this engineered male cow is A*A* and XY¹. Alternatively, the male cow is engineered to harbor one or more first sequence variants of a gene on the Y chromosome. The genotype of this engineered male cow is A*A* and XY¹. In this engineered male cow, the RNA trans-splicing process cannot occur. This engineered male cow can be bred with any lines of wildtype female cow to generate single sex offspring, e.g., a female cow.

The genotype of wildtype female cow is AA and XX, thus, when crossing with the engineered male cow A*A* and XY¹, male offspring have the genotype of A*A and XY¹ and female offspring have the genotype of A*A and XX. In some instances, the expression of the first transgenic protein occurs when the RNA trans-splicing process occurs at both 3′ splicing site and 5′ splicing site. Because Y¹ carries one or more expression(s) cassette for RNA trans-splicing process with wildtype sequence in the “A” chromosome to express the first transgenic protein, e.g., a toxin, male offspring express toxin protein and not viable. In some embodiments, translation of the first transgenic proteins, but not the second transgenic protein, is initiated when RNA trans-splicing occurs at both the first splice site and the second splice site. In some embodiments, translation of the first transgenic protein and the second transgenic protein is initiated when RNA trans-splicing occurs only at the first splice site. In some embodiments, expression of the first transgenic protein makes the male offspring visually identifiable, for example if the first transgenic protein comprises a fluorescent protein such as one or more of a green fluorescent protein (GFP), yellow fluorescent protein (YFP), red fluorescent protein (RFP), blue fluorescent protein (BFP), cyan fluorescent protein (CFP), and orange fluorescent protein (OFP). Thus, generation of single sex offspring, e.g., a female cow, is achieved via RNA trans-splicing process to express the transgenic protein, e.g., a toxin. This RNA trans-splicing system for generation of single sex offspring can be applied to other animals, all of which are compatible with methods of the present disclosure and contemplated herein. Examples of animal include, but are not limited to mammals, e.g., a cow, a mouse, a rat, a rabbit, a guinea pig, a bovine, a chimpanzee, a sheep, a goat, and a non-human primate.

In another aspect, the present disclosure provides an engineered mammal, e.g., a cow, for generation of single sex offspring, e.g., a male cow. In some instances, a male cow in the parental generation is engineered to harbor one or more expression cassette(s) for RNA trans-splicing process, wherein one or more expression cassettes comprising a nucleic acid sequence encoding a replicon RNA (repRNA), comprising a nucleic acid sequence encoding two or more transgenic proteins, two or more splice sites, and one or more introns, wherein the two or more transgenic proteins comprise a first transgenic protein and a second transgenic protein; wherein a 5′ end of the nucleic acid sequence encoding the first transgenic protein comprises a first splice site and a 3′ end of the nucleic acid sequence encoding the first transgenic protein comprises a second splice site; wherein at least one of the one or more introns does not comprise a stop codon; and wherein at least one of the one or more introns comprises a sequence that is complementary to one or more second sequence variants of the gene. In some embodiments, translation of the first transgenic protein, but not the second transgenic protein, is initiated when RNA trans-splicing occurs at both the first splice site and the second splice site. In some embodiments, translation of each of the first transgenic protein and the second transgenic protein is initiated when RNA trans-splicing occurs only at the first splice site. In some embodiments, the first transgenic protein is located on a 5′ end of the second transgenic protein. In some embodiments, the first splice site is an acceptor splice site. In some embodiments, the second splice site is a donor splice site. In some embodiments, the first transgenic protein is different than the second transgenic protein. In some embodiments, the first transgenic protein is a reporter gene. In some embodiments, the first transgenic protein is a toxin. In some embodiments, the second transgenic protein is a cognate toxin inhibitor. In some embodiments, the one or more expression cassettes further comprises a second nucleic acid sequence encoding a Cas polypeptide linked to an RNA Binding Protein (RBP) and a third nucleic acid sequence encoding a guide RNA (gRNA) capable of directing one or more CRISPR RNA-guided complexes encoded by the one or more expression cassettes to one or more second sequence variants of the gene. In some embodiments, the one or more introns comprises a first intron located on the 5′ end of the first transgenic protein, and wherein the first intron comprises a first RBP-binding hairpin. In some embodiments, the one or more introns comprises a second intron located on the 3′ end of the first transgenic protein, and wherein the second intron comprises a second RBP-binding hairpin. In some embodiments, a second RBP-binding hairpin is on a nucleic acid sequence located on a 3′ end of the second transgenic protein. In some embodiments, a second RBP-binding hairpin is on a nucleic acid sequence located on the 5′ end of the second transgenic protein. In some embodiments, the one or more introns comprises a first intron located on the 3′ end of the first transgenic protein, and wherein the first intron comprises a first RBP-binding hairpin. In some instances, a male cow in the parental generation is engineered to harbor (I) one or more expression cassettes comprising: a nucleic acid sequence encoding a replicon RNA (repRNA), comprising a nucleic acid sequence encoding two or more transgenic proteins, two or more splice sites, and one or more introns, wherein the two or more transgenic proteins comprise a first transgenic protein and a second transgenic protein; wherein a 5′ end of the nucleic acid sequence encoding the first transgenic protein comprises a first splice site and a 3′ end of the nucleic acid sequence encoding the first transgenic protein comprises a second splice site; wherein the first transgenic protein is located on a 5′ end of the second transgenic protein; wherein at least one of the one or more introns does not comprise a stop codon; and wherein at least one of the one or more introns comprises a sequence that is complementary to one or more second sequence variants of the gene; (II) one or more expression cassettes comprising: (i) a first nucleic acid sequence encoding a Cas polypeptide linked to an RNA Binding Protein (RBP); (ii) a second nucleic acid sequence encoding a replicon RNA (repRNA), comprising a nucleic acid sequence encoding two or more transgenic proteins, two or more splice sites, and one or more introns, wherein the two or more transgenic proteins comprise a first transgenic protein and a second transgenic protein; wherein a 5′ end of the nucleic acid sequence encoding the first transgenic protein comprises a first splice site and a 3′ end of the nucleic acid sequence encoding the first transgenic protein comprises a second splice site; and wherein at least one of the one or more introns (a) is connected to an RBP-binding hairpin, and/or (b) does not comprise a stop codon; and (iii) a third nucleic acid sequence encoding a guide RNA (gRNA) capable of directing one or more CRISPR RNA-guided complexes encoded by the one or more expression cassettes to one or more second sequence variants of the gene; or (III) one or more expression cassettes comprises (i) a first nucleic acid sequence encoding a Cas polypeptide linked to an RNA Binding Protein (RBP); (ii) a second nucleic acid sequence encoding a replicon RNA (repRNA), comprising a nucleic acid sequence encoding two or more transgenic proteins, two or more splice sites, and one or more introns, wherein the two or more transgenic proteins comprise a first transgenic protein and a second transgenic protein; wherein a 5′ end of the nucleic acid sequence encoding the first transgenic protein comprises a first splice site and a 3′ end of the nucleic acid sequence encoding the first transgenic protein comprises a second splice site; and wherein at least one of the one or more introns (a) is connected to an RBP-binding hairpin, and/or (b) does not comprise a stop codon; wherein at least one of the one or more introns comprises a sequence that is complementary to one or more second sequence variants of the gene; and (iii) a third nucleic acid sequence encoding a guide RNA (gRNA) capable of directing one or more CRISPR RNA-guided complexes encoded by the one or more expression cassettes to one or more third sequence variants of the gene, wherein the one or more second sequence variants of the gene and the one or more third sequence variants of the gene do not have sequence identity to each other. This expression cassette is integrated into the male cow chromosome. In some instances, the one or more expression cassette(s) is integrated into the X allosome (called X¹ or). In this instance, the genotype of the engineered male cow is X′Y. Further, in this instance, the male cow is engineered to harbor one or more first sequence variants of a gene, indicated as A*, wherein the one or more first sequence variants of the gene comprise one or more nucleotide sequences comprising a modified intron that is not capable of base pairing with a trans-splicing accepting gene. In some instances, both allele of the gene is modified, and the genotype of this engineered male cow is A*A* and X¹Y. In this engineered male cow, the RNA trans-splicing process cannot occur. This engineered male cow can be bred with any lines of wildtype female cow to generate single sex offspring, e.g., a female cow.

The genotype of wildtype female cow is AA and XX, thus, when crossing with the engineered male cow A*A* and X Y, male offspring have the genotype of A*A and XY and female offspring have the genotype of A*A and X′X. In some instances, the expression of the first transgenic protein occurs when the RNA trans-splicing process occurs at both 3′ splicing site and 5′ splicing site. Because X¹ carries one or more expression(s) cassette for RNA trans-splicing process with wildtype sequence in the “A” chromosome to express the first transgenic protein, e.g., a toxin, female offspring express toxin protein and not viable. In some embodiments, translation of the first transgenic protein and the second transgenic protein is initiated when RNA trans-splicing occurs only at the first splice site. In some embodiments, translation of the first transgenic protein, but not the second transgenic protein, is initiated when RNA trans-splicing occurs at both the first splice site and the second splice site. Alternatively, expression of the first transgenic protein makes the offspring visually identifiable, for example if the first transgenic protein comprises a fluorescent protein such as one or more of a green fluorescent protein (GFP), yellow fluorescent protein (YFP), red fluorescent protein (RFP), blue fluorescent protein (BFP), cyan fluorescent protein (CFP), and orange fluorescent protein (OFP). Thus, generation of single sex offspring, e.g., a male cow, is achieved via RNA trans-splicing process to express the first transgenic protein, e.g., a toxin. This RNA trans-splicing system for generation of single sex offspring can be applied to other animals, all of which are compatible with methods of the present disclosure and contemplated herein. Examples of animals include, but are not limited to mammals, e.g., a cow, a mouse, a rat, a rabbit, a guinea pig, a bovine, a chimpanzee, a sheep, a goat, and a non-human primate.

### Methods of Generation of Single Sex Offspring Via RNA Trans-Splicing Methods

In one aspect, the present disclosure provide a method of producing a single sex population of non-human vertebrate animals, the method comprising: crossing the first non-human vertebrate animal described herein with the second non-human vertebrate animal described herein, wherein a resulting progeny having a genotype comprising the one or more second sequence variants of the gene and the one or more expression cassettes is not viable; thereby producing the single sex population of non-human vertebrate animals. In some embodiments, the first non-human vertebrate animal is an engineered animal. In some embodiments, the first non-human vertebrate animal comprises: one or more first sequence variants of a gene, wherein the one or more first sequence variants of the gene comprise a modified intron that is not capable of RNA trans-splicing process and/or not capable of base pairing with a trans-splicing accepting gene; and one or more expression cassettes comprising a nucleic acid sequence encoding a replicon RNA (repRNA), comprising a nucleic acid sequence encoding two or more transgenic proteins, two or more splice sites, and one or more introns, wherein the two or more transgenic proteins comprise a first transgenic protein and a second transgenic protein; wherein a 5′ end of the nucleic acid sequence encoding the first transgenic protein comprises a first splice site and a 3′ end of the nucleic acid sequence encoding the first transgenic protein comprises a second splice site; wherein at least one of the one or more introns does not comprise a stop codon; and wherein at least one of the one or more introns comprises a sequence that is complementary to one or more second sequence variants of the gene. In some embodiments, translation of the first transgenic protein, but not the second transgenic protein, is initiated when RNA trans-splicing occurs at both the first splice site and the second splice site. In some embodiments, translation of each of the first transgenic protein and the second transgenic protein is initiated when RNA trans-splicing occurs only at the first splice site. In some embodiments, the first transgenic protein is located on a 5′ end of the second transgenic protein. In some embodiments, the first splice site is an acceptor splice site. In some embodiments, the second splice site is a donor splice site. In some embodiments, the first transgenic protein is different than the second transgenic protein. In some embodiments, the first transgenic protein is a reporter gene. In some embodiments, the first transgenic protein is a toxin. In some embodiments, the second transgenic protein is a cognate toxin inhibitor. In some embodiments, the one or more expression cassettes further comprises a second nucleic acid sequence encoding a Cas polypeptide linked to an RNA Binding Protein (RBP) and a third nucleic acid sequence encoding a guide RNA (gRNA) capable of directing one or more CRISPR RNA-guided complexes encoded by the one or more expression cassettes to one or more second sequence variants of the gene. In some embodiments, the one or more introns comprises a first intron located on the 5′ end of the first transgenic protein, and wherein the first intron comprises a first RBP-binding hairpin. In some embodiments, the one or more introns comprises a second intron located on the 3′ end of the first transgenic protein, and wherein the second intron comprises a second RBP-binding hairpin. In some embodiments, a second RBP-binding hairpin is on a nucleic acid sequence located on a 3′ end of the second transgenic protein. In some embodiments, a second RBP-binding hairpin is on a nucleic acid sequence located on the 5′ end of the second transgenic protein. In some embodiments, the one or more introns comprises a first intron located on the 3′ end of the first transgenic protein, and wherein the first intron comprises a first RBP-binding hairpin. In some embodiments, the first non-human vertebrate animal comprises: one or more first sequence variants of a gene, wherein the one or more first sequence variants of the gene comprise a modified intron that is not capable of RNA trans-splicing process and/or not capable of base pairing with a trans-splicing accepting gene; and (I) one or more expression cassettes comprising: a nucleic acid sequence encoding a replicon RNA (repRNA), comprising a nucleic acid sequence encoding two or more transgenic proteins, two or more splice sites, and one or more introns, wherein the two or more transgenic proteins comprise a first transgenic protein and a second transgenic protein; wherein a 5′ end of the nucleic acid sequence encoding the first transgenic protein comprises a first splice site and a 3′ end of the nucleic acid sequence encoding the first transgenic protein comprises a second splice site; wherein the first transgenic protein is located on a 5′ end of the second transgenic protein; wherein at least one of the one or more introns does not comprise a stop codon; and wherein at least one of the one or more introns comprises a sequence that is complementary to one or more second sequence variants of the gene; (II) one or more expression cassettes comprising: (i) a first nucleic acid sequence encoding a Cas polypeptide linked to an RNA Binding Protein (RBP); (ii) a second nucleic acid sequence encoding a replicon RNA (repRNA), comprising a nucleic acid sequence encoding two or more transgenic proteins, two or more splice sites, and one or more introns, wherein the two or more transgenic proteins comprise a first transgenic protein and a second transgenic protein; wherein a 5′ end of the nucleic acid sequence encoding the first transgenic protein comprises a first splice site and a 3′ end of the nucleic acid sequence encoding the first transgenic protein comprises a second splice site; and wherein at least one of the one or more introns (a) is connected to an RBP-binding hairpin, and/or (b) does not comprise a stop codon; and (iii) a third nucleic acid sequence encoding a guide RNA (gRNA) capable of directing one or more CRISPR RNA-guided complexes encoded by the one or more expression cassettes to one or more second sequence variants of the gene; or (III) one or more expression cassettes comprises (i) a first nucleic acid sequence encoding a Cas polypeptide linked to an RNA Binding Protein (RBP); (ii) a second nucleic acid sequence encoding a replicon RNA (repRNA), comprising a nucleic acid sequence encoding two or more transgenic proteins, two or more splice sites, and one or more introns, wherein the two or more transgenic proteins comprise a first transgenic protein and a second transgenic protein; wherein a 5′ end of the nucleic acid sequence encoding the first transgenic protein comprises a first splice site and a 3′ end of the nucleic acid sequence encoding the first transgenic protein comprises a second splice site; and wherein at least one of the one or more introns (a) is connected to an RBP-binding hairpin, and/or (b) does not comprise a stop codon; wherein at least one of the one or more introns comprises a sequence that is complementary to one or more second sequence variants of the gene; and (iii) a third nucleic acid sequence encoding a guide RNA (gRNA) capable of directing one or more CRISPR RNA-guided complexes encoded by the one or more expression cassettes to one or more third sequence variants of the gene, wherein the one or more second sequence variants of the gene and the one or more third sequence variants of the gene do not have sequence identity to each other.

In some embodiments, the second non-human vertebrate animal comprises one or more second sequence variants of a gene. In some embodiments, the one or more second sequence variants of a gene comprises an intron that is capable of RNA-trans-splicing process. In some embodiments, the second non-human vertebrate animal comprises a wildtype animal. In some embodiments, the second non-human vertebrate animal further comprises one or more third sequence variants of a gene. In some embodiments, the one or more third sequence variants of a gene comprises an intron that is capable of RNA-trans-splicing process. In some embodiments, translation of the first transgenic protein, but not the second transgenic protein, is initiated when RNA trans-splicing occurs at both the first splice site and the second splice site. In some embodiments, translation of each of the first transgenic protein and the second transgenic protein is initiated when RNA trans-splicing occurs only at the first splice site. By crossing the engineered animal as described in the present disclosure, generation of single sex offspring can be achieved. In some embodiments, the single sex population of non-human vertebrate animals are not genetically engineered. In some embodiments, each of the non-human vertebrate animals of the single sex population of non-human vertebrate animals are not genetically engineered.

In some embodiments, translation of the first transgenic protein, but not the second transgenic protein, is initiated when RNA trans-splicing occurs at both the first splice site and the second splice site. In some embodiments, translation of each of the first transgenic protein and the second transgenic protein is initiated when RNA trans-splicing occurs only at the first splice site. In some embodiments, the first transgenic protein is located on a 5′ end of the second transgenic protein. In some embodiments, the first splice site is an acceptor splice site. In some embodiments, the second splice site is a donor splice site. In some embodiments, the first transgenic protein is different than the second transgenic protein. In some embodiments, the first transgenic protein is a reporter gene. In some embodiments, the first transgenic protein is a toxin. In some embodiments, the toxin comprises a nuclease or a protease. In some embodiments, the nuclease comprises a Barnase or an RNase. In some embodiments, the protease comprises a caspase, trypsin, chymotrypsin, or papain. In some embodiments, the second transgenic protein is a cognate toxin inhibitor. In some embodiments, the first transgenic protein is a Barnase or a variant thereof, and the second transgenic protein is a Barstar or a variant thereof. In some embodiments, the first transgenic protein is an RNase, and the second transgenic protein is an RNase inhibitor. In some embodiments, the first transgenic protein is a restriction endonuclease, and the second transgenic protein is In some embodiments, (i) the first transgenic protein is a restriction endonuclease, and the second transgenic protein is a restriction endonuclease inhibitor; (ii) the first transgenic protein is a diphtheria, and the second transgenic protein is a diphtheria antitoxin; (iii) the first transgenic protein is a ricin, and the second transgenic protein is a ricin inhibitor; (iv) the first transgenic protein is an abrin, and the second transgenic protein is an abrin inhibitor; (v) the first transgenic protein is a pokeweed antiviral protein, and the second transgenic protein is a pokeweed antiviral protein inhibitor; (vi) the first transgenic protein is a caspase, and the second transgenic protein is a caspase inhibitor; (vii) the first transgenic protein is a trypsin, and the second transgenic protein is a trypsin inhibitor; (viii) the first transgenic protein is a chymotrypsin, and the second transgenic protein is a chymotrypsin inhibitor; or (ix) the first transgenic protein is a papain, and the second transgenic protein is a papain inhibitor. Other pairs of toxin and cognate toxin inhibitor can be used with the expression cassettes, compositions, or methods described herein. In some embodiments, the one or more expression cassettes further comprises a second nucleic acid sequence encoding a Cas polypeptide linked to an RNA Binding Protein (RBP) and a third nucleic acid sequence encoding a guide RNA (gRNA) capable of directing one or more CRISPR RNA-guided complexes encoded by the one or more expression cassettes to one or more second sequence variants of the gene. In some embodiments, the one or more introns comprises a first intron located on the 5′ end of the first transgenic protein, and wherein the first intron comprises a first RBP-binding hairpin. In some embodiments, the one or more introns comprises a second intron located on the 3′ end of the first transgenic protein, and wherein the second intron comprises a second RBP-binding hairpin. In some embodiments, a second RBP-binding hairpin is on a nucleic acid sequence located on a 3′ end of the second transgenic protein. In some embodiments, a second RBP-binding hairpin is on a nucleic acid sequence located on the 5′ end of the second transgenic protein. In some embodiments, the one or more introns comprises a first intron located on the 3′ end of the first transgenic protein, and wherein the first intron comprises a first RBP-binding hairpin. In some embodiments, the one or more expression cassettes comprise a promoter that is expressed during embryogenesis. In some embodiments, the one or more expression cassettes comprises an inducible promoter. In some embodiments, the gene is a non-essential gene. In some embodiments, the gene is an essential gene. In some embodiments, the gene is expressed in an embryo. In some embodiments, the gene is a housekeeping gene that is constitutively expressed. In some embodiments, the non-human vertebrate animal is selected from the group consisting of cow, mouse, rat, rabbit, guinea pig, chicken, fish, bird, reptile, camelid, bovine, chimpanzee, sheep, goat, and non-human primate. In some embodiments, the one or more second sequence variants of the gene do not have sequence identity to the one or more first sequence variant of the gene. In some embodiments, the one or more second sequence variants of the gene have sequence identity to a wildtype sequence of the gene. In some embodiments, the RNA trans-splicing process is spliceosome mediated. In some embodiments, the RNA trans-splicing process is ribozyme mediated. In some embodiments, the Cas polypeptide is a Cas endonuclease. In some embodiments, the Cas endonuclease is a class II Cas endonuclease. In some embodiments, the Cas endonuclease is a type II, type III, or type VI Cas endonuclease. In some embodiments, the Cas endonuclease is an RNA-guided RNA endonuclease. In some embodiments, the Cas endonuclease is selected from the group consisting of Cas9, Cas13, Csm/Cmr, Cas7-11, DisCas7-11, and Cas12a. In some embodiments, the Cas7-11 is Cas7-11a, Cas7-11b, Cas7-11c, or Cas7-11d. In some embodiments, the Cas polypeptide is a variant of the Cas endonuclease. In some embodiments, the Cas polypeptide is an inactive form of the Cas endonuclease. In some embodiments, the Cas polypeptide binds to a polynucleotide but does not cleave the polynucleotide. In some embodiments, the Cas polypeptide is a deactivated Cas13 (dCas13) or a deactivated dDisCas7-11 (dDisCas7-11). In some embodiments, the Cas polypeptide is a dCas13a, dCas13b, dCas13c, or dCas13d. In some embodiments, the Cas polypeptide is a variant of a Prevotella sp. Cas13b (PspCas13b). In some embodiments, the RBP is selected from the group consisting of MS2 coat protein (MCP), PP7 bacteriophage coat protein, small RNA phage PRR1, and RNA bacteriophages QB coat protein. In some embodiments, the number of the RBP-binding hairpins is at least 1, at least 2, at least 3, at least 4, at least 5, at least 6, at least 7, at least 8, at least 9, or at least 10. In some embodiments, the one or more CRISPR RNA-guided complexes comprise the guide RNA, the Cas polypeptide, the repRNA, or a combination thereof. In some embodiments, the RNA trans-splicing process at 3′ splicing site results in translation of both the first transgenic protein and the second transgenic protein. In some embodiments, the RNA trans-splicing process at both 3′ splicing site and 5′ splicing site results in translation of the first transgenic protein but not the second transgenic protein. In some embodiments, the gene is an autosomal gene. In some embodiments, the gene is an allosomal gene. In some embodiments, the one or more expression cassettes is located on an autosomal chromosome. In some embodiments, the one or more expression cassettes is integrated into an autosome. In some embodiments, the one or more expression cassettes is located on an allosomal chromosome. In some embodiments, the one or more expression cassettes is integrated into an allosome.

### Single Sex Offspring Generated Via RNA Trans-Splicing Methods

In one aspect, the present disclosure provides non-human vertebrate animals having a modified genotype comprising heterozygous autosomes, wherein one of the heterozygous autosomes comprises one or more first sequence variants of a gene, wherein the one or more first sequence variants of the gene comprises one or more nucleotide sequences comprising a modified intron that is not capable of base pairing with a trans-splicing accepting gene; and wherein another one of the heterozygous autosomes comprises a wildtype sequence variant of the gene. In some embodiments, the trans-splicing accepting gene is a non-essential gene. In some embodiments, the trans-splicing accepting gene is an essential gene. In some embodiments, the trans-splicing accepting gene is expressed in an embryo. In some embodiments, the trans-splicing accepting gene is a housekeeping gene that is constitutively expressed. In some embodiments, the non-human vertebrate animal is selected from the group consisting of cow, mouse, rat, rabbit, guinea pig, chicken, fish, bird, reptile, camelid, bovine, chimpanzee, sheep, goat, and non-human primate.

In another aspect, the present disclosure provides non-human vertebrate animals having a modified genotype comprising heterozygous allosomes, wherein one of the heterozygous allosomes comprises one or more first sequence variants of a gene, wherein the one or more first sequence variants of the gene comprises one or more nucleotide sequences comprising a modified intron that is not capable of base pairing with a trans-splicing accepting gene; and wherein another one of the heterozygous allosomes comprises a wildtype sequence variant of the gene. In some embodiments, the trans-splicing accepting gene is a non-essential gene. In some embodiments, the trans-splicing accepting gene is an essential gene. In some embodiments, the trans-splicing accepting gene is expressed in an embryo. In some embodiments, the trans-splicing accepting gene is a housekeeping gene that is constitutively expressed. In some embodiments, the trans-splicing accepting gene is Rictor. In some embodiments, the non-human vertebrate animal is selected from the group consisting of cow, mouse, rat, rabbit, guinea pig, chicken, fish, bird, reptile, camelid, bovine, chimpanzee, sheep, goat, and non-human primate.

### Single Sex Offspring in Poultry

In one aspect, the present disclosure provides methods and compositions utilizing an RNA trans-splicing system that can (1) differentiate between on-target trans-splicing event and off-target trans-splicing event and/or (2) express the first transgenic protein, e.g., a toxin, when trans-splicing occurs at both the first splice site and the second splice site, e.g., both 3′ splice site and 5′ splice site, to generate single sex offspring in animal such as a chicken. In some instances, the second transgenic protein is designed to function as a safety switch when off-target trans-splicing events occur.

In some instances, the single sex offspring is a female offspring. For example, the Z allosome (called Z¹ allosome) of the engineered female chicken in the parental generation carries one or more expression cassette(s) for RNA trans-splicing process, wherein the one or more expression cassette(s) comprise a nucleic acid sequence encoding a replicon RNA (repRNA), comprising a nucleic acid sequence encoding two or more transgenic proteins, two or more splice sites, and one or more introns, wherein the two or more transgenic proteins comprise a first transgenic protein and a second transgenic protein; wherein a 5′ end of the nucleic acid sequence encoding the first transgenic protein comprises a first splice site and a 3′ end of the nucleic acid sequence encoding the first transgenic protein comprises a second splice site; wherein at least one of the one or more introns does not comprise a stop codon; and wherein at least one of the one or more introns comprises a sequence that is complementary to one or more second sequence variants of the gene. In some embodiments, translation of the first transgenic protein, but not the second transgenic protein, is initiated when RNA trans-splicing occurs at both the first splice site and the second splice site. In some embodiments, translation of each of the first transgenic protein and the second transgenic protein is initiated when RNA trans-splicing occurs only at the first splice site. In some embodiments, the first transgenic protein is located on a 5′ end of the second transgenic protein. In some embodiments, the first splice site is an acceptor splice site. In some embodiments, the second splice site is a donor splice site. In some embodiments, the first transgenic protein is different than the second transgenic protein. In some embodiments, the first transgenic protein is a reporter gene. In some embodiments, the first transgenic protein is a toxin. In some embodiments, the second transgenic protein is a cognate toxin inhibitor. In some embodiments, the one or more expression cassettes further comprises a second nucleic acid sequence encoding a Cas polypeptide linked to an RNA Binding Protein (RBP) and a third nucleic acid sequence encoding a guide RNA (gRNA) capable of directing one or more CRISPR RNA-guided complexes encoded by the one or more expression cassettes to one or more second sequence variants of the gene. In some embodiments, the one or more introns comprises a first intron located on the 5′ end of the first transgenic protein, and wherein the first intron comprises a first RBP-binding hairpin. In some embodiments, the one or more introns comprises a second intron located on the 3′ end of the first transgenic protein, and wherein the second intron comprises a second RBP-binding hairpin. In some embodiments, a second RBP-binding hairpin is on a nucleic acid sequence located on a 3′ end of the second transgenic protein. In some embodiments, a second RBP-binding hairpin is on a nucleic acid sequence located on the 5′ end of the second transgenic protein. In some embodiments, the one or more Introns comprises a first intron located on the 3′ end of the first transgenic protein, and wherein the first intron comprises a first RBP-binding hairpin. In some instances, the Z allosome (called Z¹ allosome) of the engineered female chicken in the parental generation carries one or more expression cassette(s) for RNA trans-splicing process, wherein (I) one or more expression cassettes comprising: a nucleic acid sequence encoding a replicon RNA (repRNA), comprising a nucleic acid sequence encoding two or more transgenic proteins, two or more splice sites, and one or more introns, wherein the two or more transgenic proteins comprise a first transgenic protein and a second transgenic protein; wherein a 5′ end of the nucleic acid sequence encoding the first transgenic protein comprises a first splice site and a 3′ end of the nucleic acid sequence encoding the first transgenic protein comprises a second splice site; wherein the first transgenic protein is located on a 5′ end of the second transgenic protein; wherein at least one of the one or more introns does not comprise a stop codon; and wherein at least one of the one or more introns comprises a sequence that is complementary to one or more second sequence variants of the gene; (II) one or more expression cassettes comprising: (i) a first nucleic acid sequence encoding a Cas polypeptide linked to an RNA Binding Protein (RBP); (ii) a second nucleic acid sequence encoding a replicon RNA (repRNA), comprising a nucleic acid sequence encoding two or more transgenic proteins, two or more splice sites, and one or more introns, wherein the two or more transgenic proteins comprise a first transgenic protein and a second transgenic protein; wherein a 5′ end of the nucleic acid sequence encoding the first transgenic protein comprises a first splice site and a 3′ end of the nucleic acid sequence encoding the first transgenic protein comprises a second splice site; and wherein at least one of the one or more introns (a) is connected to an RBP-binding hairpin, and/or (b) does not comprise a stop codon; and (iii) a third nucleic acid sequence encoding a guide RNA (gRNA) capable of directing one or more CRISPR RNA-guided complexes encoded by the one or more expression cassettes to one or more second sequence variants of the gene; or (III) one or more expression cassettes comprises (i) a first nucleic acid sequence encoding a Cas polypeptide linked to an RNA Binding Protein (RBP); (ii) a second nucleic acid sequence encoding a replicon RNA (repRNA), comprising a nucleic acid sequence encoding two or more transgenic proteins, two or more splice sites, and one or more introns, wherein the two or more transgenic proteins comprise a first transgenic protein and a second transgenic protein; wherein a 5′ end of the nucleic acid sequence encoding the first transgenic protein comprises a first splice site and a 3′ end of the nucleic acid sequence encoding the first transgenic protein comprises a second splice site; and wherein at least one of the one or more introns (a) is connected to an RBP-binding hairpin, and/or (b) does not comprise a stop codon; wherein at least one of the one or more introns comprises a sequence that is complementary to one or more second sequence variants of the gene; and (iii) a third nucleic acid sequence encoding a guide RNA (gRNA) capable of directing one or more CRISPR RNA-guided complexes encoded by the one or more expression cassettes to one or more third sequence variants of the gene, wherein the one or more second sequence variants of the gene and the one or more third sequence variants of the gene do not have sequence identity to each other. Further, the female chicken is engineered to harbor one or more first sequence variants of a gene, indicated as A*, wherein the one or more first sequence variants of the gene comprise one or more nucleotide sequences comprising a modified intron that is not capable of base pairing with a trans-splicing accepting gene. In this instance, both alleles of the gene are modified, and the genotype of this engineered female chicken is A*A* and Z¹W. In this engineered female chicken, the RNA trans-splicing process cannot occur. This engineered female chicken can be bred with any lines of wildtype male chicken to generate single sex offspring, e.g., a female layer hen. In some embodiments, the genotype of female offspring is A*A and ZW and viable while the genotype of male offspring is A*A and Z¹Z, which is not viable. In some embodiments, the genotype of female offspring is A*A and ZW and does not express a visual marker (e.g., a fluorescent protein such as a green fluorescent protein) while the genotype of male offspring is A*A and Z¹Z, which expresses the visual marker (e.g., a green fluorescent protein). This RNA trans-splicing system for generation of single sex offspring can be applied to other animals, all of which are compatible with methods of the present disclosure and contemplated herein. Examples of animals include, but are not limited to chicken, bird, and reptile.

In another aspect, the present disclosure provides methods and compositions utilizing RNA trans-splicing system that can (1) differentiate between on-target trans-splicing event and off-target trans-splicing event and/or (2) express the first transgenic protein, e.g., a toxin, when trans-splicing occurs at both the first splice site and the second splice site, e.g., both 3′ splice site and 5′ splice site, to generate single sex offspring in animal such as a chicken. In some instances, the single sex offspring is a female offspring. For example, the Z allosome (called allosome) of the engineered female chicken in the parental generation carries one or more expression cassette(s) for RNA trans-splicing process, wherein the one or more expression cassette(s) comprise a nucleic acid sequence encoding a replicon RNA (repRNA), comprising a nucleic acid sequence encoding two or more transgenic proteins, two or more splice sites, and one or more introns, wherein the two or more transgenic proteins comprise a first transgenic protein and a second transgenic protein; wherein a 5′ end of the nucleic acid sequence encoding the first transgenic protein comprises a first splice site and a 3′ end of the nucleic acid sequence encoding the first transgenic protein comprises a second splice site; wherein at least one of the one or more introns does not comprise a stop codon; and wherein at least one of the one or more introns comprises a sequence that is complementary to one or more second sequence variants of the gene. In some embodiments, translation of the first transgenic protein, but not the second transgenic protein, is initiated when RNA trans-splicing occurs at both the first splice site and the second splice site. In some embodiments, translation of each of the first transgenic protein and the second transgenic protein is initiated when RNA trans-splicing occurs only at the first splice site. In some embodiments, the first transgenic protein is located on a 5′ end of the second transgenic protein. In some embodiments, the first splice site is an acceptor splice site. In some embodiments, the second splice site is a donor splice site. In some embodiments, the first transgenic protein is different than the second transgenic protein. In some embodiments, the first transgenic protein is a reporter gene. In some embodiments, the first transgenic protein is a toxin. In some embodiments, the second transgenic protein is a cognate toxin inhibitor. In some embodiments, the one or more expression cassettes further comprises a second nucleic acid sequence encoding a Cas polypeptide linked to an RNA Binding Protein (RBP) and a third nucleic acid sequence encoding a guide RNA (gRNA) capable of directing one or more CRISPR RNA-guided complexes encoded by the one or more expression cassettes to one or more second sequence variants of the gene. In some embodiments, the one or more introns comprises a first intron located on the 5′ end of the first transgenic protein, and wherein the first intron comprises a first RBP-binding hairpin. In some embodiments, the one or more introns comprises a second intron located on the 3′ end of the first transgenic protein, and wherein the second intron comprises a second RBP-binding hairpin. In some embodiments, a second RBP-binding hairpin is on a nucleic acid sequence located on a 3′ end of the second transgenic protein. In some embodiments, a second RBP-binding hairpin is on a nucleic acid sequence located on the 5′ end of the second transgenic protein. In some embodiments, the one or more introns comprises a first intron located on the 3′ end of the first transgenic protein, and wherein the first intron comprises a first RBP-binding hairpin. In some instances, the Z allosome (called Z¹ allosome) of the engineered female chicken in the parental generation carries one or more expression cassette(s) for RNA trans-splicing process, wherein (I) one or more expression cassettes comprising: a nucleic acid sequence encoding a replicon RNA (repRNA), comprising a nucleic acid sequence encoding two or more transgenic proteins, two or more splice sites, and one or more introns, wherein the two or more transgenic proteins comprise a first transgenic protein and a second transgenic protein; wherein a 5′ end of the nucleic acid sequence encoding the first transgenic protein comprises a first splice site and a 3′ end of the nucleic acid sequence encoding the first transgenic protein comprises a second splice site; wherein the first transgenic protein is located on a 5′ end of the second transgenic protein; wherein at least one of the one or more introns does not comprise a stop codon; and wherein at least one of the one or more introns comprises a sequence that is complementary to one or more second sequence variants of the gene; (II) one or more expression cassettes comprising: (i) a first nucleic acid sequence encoding a Cas polypeptide linked to an RNA Binding Protein (RBP); (ii) a second nucleic acid sequence encoding a replicon RNA (repRNA), comprising a nucleic acid sequence encoding two or more transgenic proteins, two or more splice sites, and one or more introns, wherein the two or more transgenic proteins comprise a first transgenic protein and a second transgenic protein; wherein a 5′ end of the nucleic acid sequence encoding the first transgenic protein comprises a first splice site and a 3′ end of the nucleic acid sequence encoding the first transgenic protein comprises a second splice site; and wherein at least one of the one or more introns (a) is connected to an RBP-binding hairpin, and/or (b) does not comprise a stop codon; and (iii) a third nucleic acid sequence encoding a guide RNA (gRNA) capable of directing one or more CRISPR RNA-guided complexes encoded by the one or more expression cassettes to one or more second sequence variants of the gene; or (III) one or more expression cassettes comprises (i) a first nucleic acid sequence encoding a Cas polypeptide linked to an RNA Binding Protein (RBP); (ii) a second nucleic acid sequence encoding a replicon RNA (repRNA), comprising a nucleic acid sequence encoding two or more transgenic proteins, two or more splice sites, and one or more introns, wherein the two or more transgenic proteins comprise a first transgenic protein and a second transgenic protein; wherein a 5′ end of the nucleic acid sequence encoding the first transgenic protein comprises a first splice site and a 3′ end of the nucleic acid sequence encoding the first transgenic protein comprises a second splice site; and wherein at least one of the one or more introns (a) is connected to an RBP-binding hairpin, and/or (b) does not comprise a stop codon; wherein at least one of the one or more introns comprises a sequence that is complementary to one or more second sequence variants of the gene; and (iii) a third nucleic acid sequence encoding a guide RNA (gRNA) capable of directing one or more CRISPR RNA-guided complexes encoded by the one or more expression cassettes to one or more third sequence variants of the gene, wherein the one or more second sequence variants of the gene and the one or more third sequence variants of the gene do not have sequence identity to each other. In some embodiments, translation of a first transgenic protein of the two or more transgenic proteins is initiated when RNA trans-splicing occurs at both the first splice site and the second splice site. In some embodiments, the first transgenic protein is a toxin. In some embodiments, the second transgenic protein is a cognate toxin inhibitor. In some embodiments, the first transgenic protein is a reporter gene. The allosome of the female chicken is further engineered to harbor one or more first sequence variants of a gene, wherein the one or more first sequence variants of the gene comprise one or more nucleotide sequences comprising a modified intron that is not capable of base pairing with a trans-splicing accepting gene. In this instance, the genotype of this engineered female chicken is W. In this engineered female chicken, the RNA trans-splicing process cannot occur. This engineered female chicken can be bred with any lines of wildtype male chicken to generate single sex offspring, e.g., a female layer hen. The genotype of female offspring is ZW and viable, while the genotype of male offspring is Z, which is not viable. Furthermore, the female ZW offspring is not genetically modified. This RNA trans-splicing system for generation of single sex offspring can be applied to other animals, all of which are compatible with methods of the present disclosure and contemplated herein. Examples of animals include, but are not limited to chicken, bird, and reptile.

### Single Sex Offspring in Mammals

In one aspect, the present disclosure provides methods and compositions utilizing RNA trans-splicing system described herein to express toxin to generate single sex offspring in animal such as cows or pigs. In some instances, the single sex offspring is a female offspring. For example, the Y allosome (called Y¹ allosome) of the engineered male cow in parental generation carries one or more expression cassette(s) for RNA trans-splicing process, wherein the one or more expression cassette(s) comprise a nucleic acid sequence encoding a replicon RNA (repRNA), comprising a nucleic acid sequence encoding two or more transgenic proteins, two or more splice sites, and one or more introns, wherein the two or more transgenic proteins comprise a first transgenic protein and a second transgenic protein; wherein a 5′ end of the nucleic acid sequence encoding the first transgenic protein comprises a first splice site and a 3′ end of the nucleic acid sequence encoding the first transgenic protein comprises a second splice site; wherein at least one of the one or more introns does not comprise a stop codon; and wherein at least one of the one or more introns comprises a sequence that is complementary to one or more second sequence variants of the gene. In some embodiments, translation of the first transgenic protein, but not the second transgenic protein, is initiated when RNA trans-splicing occurs at both the first splice site and the second splice site. In some embodiments, translation of each of the first transgenic protein and the second transgenic protein is initiated when RNA trans-splicing occurs only at the first splice site. In some embodiments, the first transgenic protein is located on a 5′ end of the second transgenic protein. In some embodiments, the first splice site is an acceptor splice site. In some embodiments, the second splice site is a donor splice site. In some embodiments, the first transgenic protein is different than the second transgenic protein. In some embodiments, the first transgenic protein is a reporter gene. In some embodiments, the first transgenic protein is a toxin. In some embodiments, the second transgenic protein is a cognate toxin inhibitor. In some embodiments, the one or more expression cassettes further comprises a second nucleic acid sequence encoding a Cas polypeptide linked to an RNA Binding Protein (RBP) and a third nucleic acid sequence encoding a guide RNA (gRNA) capable of directing one or more CRISPR RNA-guided complexes encoded by the one or more expression cassettes to one or more second sequence variants of the gene. In some embodiments, the one or more introns comprises a first intron located on the 5′ end of the first transgenic protein, and wherein the first intron comprises a first RBP-binding hairpin. In some embodiments, the one or more introns comprises a second intron located on the 3′ end of the first transgenic protein, and wherein the second intron comprises a second RBP-binding hairpin. In some embodiments, a second RBP-binding hairpin is on a nucleic acid sequence located on a 3′ end of the second transgenic protein. In some embodiments, a second RBP-binding hairpin is on a nucleic acid sequence located on the 5′ end of the second transgenic protein. In some embodiments, the one or more introns comprises a first intron located on the 3′ end of the first transgenic protein, and wherein the first intron comprises a first RBP-binding hairpin. In some embodiments, the Y allosome (called Y¹ allosome) of the engineered male cow in parental generation carries one or more expression cassette(s) for RNA trans-splicing process, wherein (I) one or more expression cassettes comprising: a nucleic acid sequence encoding a replicon RNA (repRNA), comprising a nucleic acid sequence encoding two or more transgenic proteins, two or more splice sites, and one or more introns, wherein the two or more transgenic proteins comprise a first transgenic protein and a second transgenic protein; wherein a 5′ end of the nucleic acid sequence encoding the first transgenic protein comprises a first splice site and a 3′ end of the nucleic acid sequence encoding the first transgenic protein comprises a second splice site; wherein the first transgenic protein is located on a 5′ end of the second transgenic protein; wherein at least one of the one or more introns does not comprise a stop codon; and wherein at least one of the one or more introns comprises a sequence that is complementary to one or more second sequence variants of the gene; (II) one or more expression cassettes comprising: (i) a first nucleic acid sequence encoding a Cas polypeptide linked to an RNA Binding Protein (RBP); (ii) a second nucleic acid sequence encoding a replicon RNA (repRNA), comprising a nucleic acid sequence encoding two or more transgenic proteins, two or more splice sites, and one or more introns, wherein the two or more transgenic proteins comprise a first transgenic protein and a second transgenic protein; wherein a 5′ end of the nucleic acid sequence encoding the first transgenic protein comprises a first splice site and a 3′ end of the nucleic acid sequence encoding the first transgenic protein comprises a second splice site; and wherein at least one of the one or more introns (a) is connected to an RBP-binding hairpin, and/or (b) does not comprise a stop codon; and (iii) a third nucleic acid sequence encoding a guide RNA (gRNA) capable of directing one or more CRISPR RNA-guided complexes encoded by the one or more expression cassettes to one or more second sequence variants of the gene; or (III) one or more expression cassettes comprises (i) a first nucleic acid sequence encoding a Cas polypeptide linked to an RNA Binding Protein (RBP); (ii) a second nucleic acid sequence encoding a replicon RNA (repRNA), comprising a nucleic acid sequence encoding two or more transgenic proteins, two or more splice sites, and one or more introns, wherein the two or more transgenic proteins comprise a first transgenic protein and a second transgenic protein; wherein a 5′ end of the nucleic acid sequence encoding the first transgenic protein comprises a first splice site and a 3′ end of the nucleic acid sequence encoding the first transgenic protein comprises a second splice site; and wherein at least one of the one or more introns (a) is connected to an RBP-binding hairpin, and/or (b) does not comprise a stop codon; wherein at least one of the one or more introns comprises a sequence that is complementary to one or more second sequence variants of the gene; and (iii) a third nucleic acid sequence encoding a guide RNA (gRNA) capable of directing one or more CRISPR RNA-guided complexes encoded by the one or more expression cassettes to one or more third sequence variants of the gene, wherein the one or more second sequence variants of the gene and the one or more third sequence variants of the gene do not have sequence identity to each other. Further, the male cow is engineered to harbor one or more first sequence variants of a gene, indicated as A*, wherein the one or more first sequence variants of the gene comprise one or more nucleotide sequences comprising a modified intron that is not capable of base pairing with a trans-splicing accepting gene. In this instance, both allele of the gene is modified, and the genotype of this engineered male cow is A*A* and XY¹. In this engineered male cow, the RNA trans-splicing process cannot occur. This engineered male cow can be bred with any lines of wildtype female cow to generate single sex offspring, e.g., a female cow. The genotype of female offspring is A*A and XX and viable while the genotype of male offspring is A*A and XY¹, which is not viable. This RNA trans-splicing system for generation of single sex offspring can be applied to other animals, all of which are compatible with methods of the present disclosure and contemplated herein. Examples of animals include, but are not limited to mammals, e.g., a cow, a mouse, a rat, a rabbit, a guinea pig, a bovine, a chimpanzee, a sheep, a goat, and a non-human primate.

In another aspect, the present disclosure provides methods and compositions utilizing RNA trans-splicing system to express toxin to generate single sex offspring in animal such as cows or pigs. In some instances, the single sex offspring is a male offspring. For example, the X allosome (called X¹ allosome) of the engineered male cow in parental generation carries one or more expression cassette(s) for RNA trans-splicing process, wherein the one or more expression cassette(s) comprise a nucleic acid sequence encoding a replicon RNA (repRNA), comprising a nucleic acid sequence encoding two or more transgenic proteins, two or more splice sites, and one or more introns, wherein the two or more transgenic proteins comprise a first transgenic protein and a second transgenic protein; wherein a 5′ end of the nucleic acid sequence encoding the first transgenic protein comprises a first splice site and a 3′ end of the nucleic acid sequence encoding the first transgenic protein comprises a second splice site; wherein at least one of the one or more introns does not comprise a stop codon; and wherein at least one of the one or more introns comprises a sequence that is complementary to one or more second sequence variants of the gene. In some embodiments, translation of the first transgenic protein, but not the second transgenic protein, is initiated when RNA trans-splicing occurs at both the first splice site and the second splice site. In some embodiments, translation of each of the first transgenic protein and the second transgenic protein is initiated when RNA trans-splicing occurs only at the first splice site. In some embodiments, the first transgenic protein is located on a 5′ end of the second transgenic protein. In some embodiments, the first splice site is an acceptor splice site. In some embodiments, the second splice site is a donor splice site. In some embodiments, the first transgenic protein is different than the second transgenic protein. In some embodiments, the first transgenic protein is a reporter gene. In some embodiments, the first transgenic protein is a toxin. In some embodiments, the second transgenic protein is a cognate toxin inhibitor. In some embodiments, the one or more expression cassettes further comprises a second nucleic acid sequence encoding a Cas polypeptide linked to an RNA Binding Protein (RBP) and a third nucleic acid sequence encoding a guide RNA (gRNA) capable of directing one or more CRISPR RNA-guided complexes encoded by the one or more expression cassettes to one or more second sequence variants of the gene. In some embodiments, the one or more introns comprises a first intron located on the 5′ end of the first transgenic protein, and wherein the first intron comprises a first RBP-binding hairpin. In some embodiments, the one or more introns comprises a second intron located on the 3′ end of the first transgenic protein, and wherein the second intron comprises a second RBP-binding hairpin. In some embodiments, a second RBP-binding hairpin is on a nucleic acid sequence located on a 3′ end of the second transgenic protein. In some embodiments, a second RBP-binding hairpin is on a nucleic acid sequence located on the 5′ end of the second transgenic protein. In some embodiments, the one or more introns comprises a first intron located on the 3′ end of the first transgenic protein, and wherein the first intron comprises a first RBP-binding hairpin. In some embodiments, the X allosome (called X¹ allosome) of the engineered male cow in parental generation carries one or more expression cassette(s) for RNA trans-splicing process, wherein (I) one or more expression cassettes comprising: a nucleic acid sequence encoding a replicon RNA (repRNA), comprising a nucleic acid sequence encoding two or more transgenic proteins, two or more splice sites, and one or more introns, wherein the two or more transgenic proteins comprise a first transgenic protein and a second transgenic protein; wherein a 5′ end of the nucleic acid sequence encoding the first transgenic protein comprises a first splice site and a 3′ end of the nucleic acid sequence encoding the first transgenic protein comprises a second splice site; wherein the first transgenic protein is located on a 5′ end of the second transgenic protein; wherein at least one of the one or more introns does not comprise a stop codon; and wherein at least one of the one or more introns comprises a sequence that is complementary to one or more second sequence variants of the gene; (II) one or more expression cassettes comprising: (i) a first nucleic acid sequence encoding a Cas polypeptide linked to an RNA Binding Protein (RBP); (ii) a second nucleic acid sequence encoding a replicon RNA (repRNA), comprising a nucleic acid sequence encoding two or more transgenic proteins, two or more splice sites, and one or more introns, wherein the two or more transgenic proteins comprise a first transgenic protein and a second transgenic protein; wherein a 5′ end of the nucleic acid sequence encoding the first transgenic protein comprises a first splice site and a 3′ end of the nucleic acid sequence encoding the first transgenic protein comprises a second splice site; and wherein at least one of the one or more introns (a) is connected to an RBP-binding hairpin, and/or (b) does not comprise a stop codon; and (iii) a third nucleic acid sequence encoding a guide RNA (gRNA) capable of directing one or more CRISPR RNA-guided complexes encoded by the one or more expression cassettes to one or more second sequence variants of the gene; or (III) one or more expression cassettes comprises (i) a first nucleic acid sequence encoding a Cas polypeptide linked to an RNA Binding Protein (RBP); (ii) a second nucleic acid sequence encoding a replicon RNA (repRNA), comprising a nucleic acid sequence encoding two or more transgenic proteins, two or more splice sites, and one or more introns, wherein the two or more transgenic proteins comprise a first transgenic protein and a second transgenic protein; wherein a 5′ end of the nucleic acid sequence encoding the first transgenic protein comprises a first splice site and a 3′ end of the nucleic acid sequence encoding the first transgenic protein comprises a second splice site; and wherein at least one of the one or more introns (a) is connected to an RBP-binding hairpin, and/or (b) does not comprise a stop codon; wherein at least one of the one or more introns comprises a sequence that is complementary to one or more second sequence variants of the gene; and (iii) a third nucleic acid sequence encoding a guide RNA (gRNA) capable of directing one or more CRISPR RNA-guided complexes encoded by the one or more expression cassettes to one or more third sequence variants of the gene, wherein the one or more second sequence variants of the gene and the one or more third sequence variants of the gene do not have sequence identity to each other. Further, the male cow is engineered to harbor one or more first sequence variants of a gene, indicated as A*, wherein the one or more first sequence variants of the gene comprise one or more nucleotide sequences comprising a modified intron that is not capable of base pairing with a trans-splicing accepting gene. In this instance, both allele of the gene is modified, and the genotype of this engineered male cow is A*A* and X¹Y. In this engineered male cow, the RNA trans-splicing process cannot occur. This engineered male cow can be bred with any lines of wildtype female cow to generate single sex offspring, e.g., a female cow. The genotype of female offspring is A*A and X¹X and not viable while the genotype of male offspring is A*A and XY, which is viable. This RNA trans-splicing system for generation of single sex offspring can be applied to other animals, all of which are compatible with methods of the present disclosure and contemplated herein. Examples of animals include, but are not limited to mammals, e.g., a cow, a mouse, a rat, a rabbit, a guinea pig, a bovine, a chimpanzee, a sheep, a goat, and a non-human primate.

### EXAMPLES

The following examples are given for the purpose of illustrating various embodiments of the invention and are not meant to limit the present invention in any fashion. The present examples, along with the methods described herein are presently representative of preferred embodiments, are exemplary, and are not intended as limitations on the scope of the invention. Changes therein and other uses which are encompassed within the spirit of the invention as defined by the scope of the claims will occur to those skilled in the art.

### Example 1: Genetic Crossing Using a Trans-Splicing Approach to Generate Single Sex Offspring

In this example, the generation of single sex female layer hens is described.

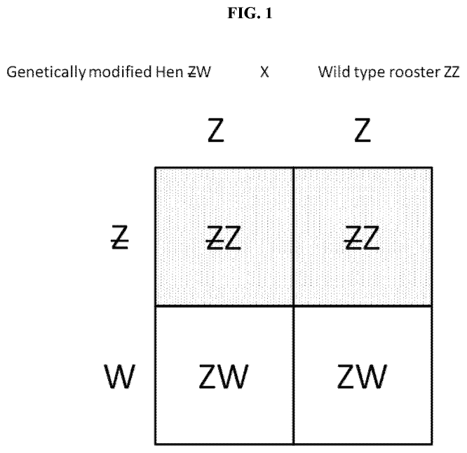

FIG. 1 shows a mating cross schematic where the parent generation of female animals, e.g., a chicken, has a modified Z chromosome (ZW), carrying (i) an expression cassette for a trans-splicing construct comprising two or more transgenes designed to be trans-spliced with the targeted pre-mRNA and (ii) a modification of the gene encoding the targeted mRNA on the modified Z chromosome such that the trans-splicing event cannot occur with the modified Z chromosome. The male animals in this cross are wildtype ZZ. For progeny of this cross, a female animal has the genotype ZW, and a male animal has the genotype Z. Male animals are conceived but they die early on in development because the inheritance of the modified chromosome from the female parent animal and a wildtype Z chromosome from the male parent animal, which, upon a trans-splicing event, result in the expression of a transgene, e.g., a toxin, capable of effectuating cell death. Accordingly, the only animals that are viable from this cross are wildtype female offspring. Note: the genetically modified chromosomes are shown in strikethrough. W is a genotype of genetically modified female animal (parent generation). ZZ is a genotype of wildtype male animal (parent generation). ZW is a genotype of a female offspring. Z is a genotype of a male offspring, which is not viable.

As shown in FIG. 1, is an allosomal gene that is modified such that the trans-splicing process cannot occur. is also engineered to express one or more trans-splicing expression cassettes encoding two or more transgenes, e.g., a first transgene encoding a toxin and a second transgene encoding the cognate toxin inhibitor of the toxin encoded by the first transgene. W indicates the engineered female parent chicken, and ZZ indicates the wildtype male parent chicken. The male chicken used in this cross can be from any chicken line. Any male offspring with a genotype Z undergoes trans-splicing events, which results in expression of transgenes. FIG. 1 shows results of this cross. Since male offspring have the Z genotype, trans-splicing events occur. With a correct trans-splicing process, the expression of the transgene, e.g., a toxin, without its cognate toxin inhibitor, occurs, thus, male offspring are not viable and generation of female offspring that are not genetically modified can be achieved.

### Example 2: Enhanced Trans-Splicing Approach Using Protein Intermediary

In this example, to improve targeting specificity of the trans-splicing construct, the use of a protein intermediary, e.g., an RNA binding framework such as CRISPR RNA-guided complexes as described by Borrajo et al “Programmable multi-kilobase RNA editing using CRISPR-mediated trans-splicing.” bioRxiv. 18 Aug. 2023, doi: 10.1101/2023.08.18.553620; Fiflis, David N et al. “Repurposing CRISPR-Cas13 systems for robust mRNA trans-splicing.” Nature communications vol. 15,1 2325. 14 Mar. 2024, doi: 10.1038/s41467-024-46172-4; and US2021/0071178A1, each of which are hereby incorporated by reference of their entities, can be utilized.

For example, a construct may comprise a CRISPR RNA-guided complex comprising deactivated Cas13 (dCas13), RNA-binding protein (RBP), guide RNA (gRNA), a replicon RNA (repRNA) that comprises an open reading frame encoding a transgenic protein, a splice site, an intron with RBP-binding hairpins, and a polyadenylation signal. An RNA binding framework enhances trans-splicing by using RNA-guided proteins to direct a repRNA to the vicinity of the targeted splice junction.

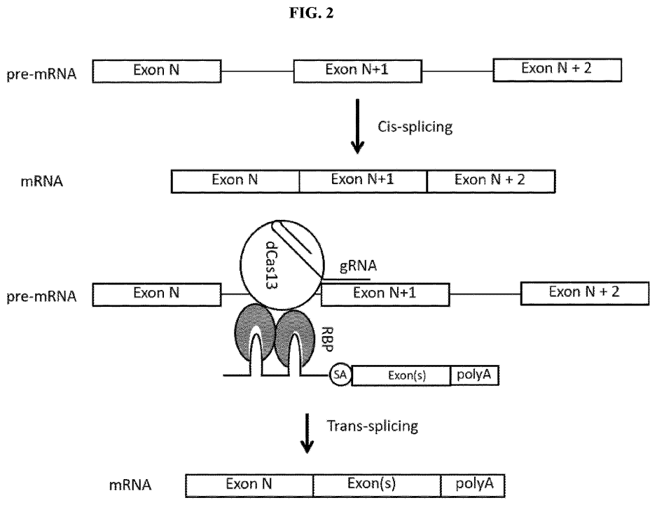

FIG. 2 shows an overview of an enhanced trans-splicing scheme via an RNA binding framework. The top part of the figure shows a cis-splicing event. The bottom part of the figure shows a trans-splicing event. In the trans-splicing process, CRISPR-mediated trans-splicing is achieved by binding the CRISPR-Cas RNP and repRNA complex to a target pre-mRNA. The dCas13-RBP binds to and blocks the cis-splicing acceptor while simultaneously recruiting the splice repRNA, thus enabling efficient and specific trans-splicing to produce a transgenic gene of interest, e.g., a toxin.

An RNA binding framework enhances trans-splicing by using RNA-guided protein complex to specifically direct a multi-kilobase replicon RNA (repRNA) to the vicinity of the targeted splice junction. Enhanced trans-splicing process is achieved using a HEPN-nuclease-deactivated Cas13 variant (dCas13) to recruit a trans-splicing repRNA and simultaneously inhibit cis-splicing by targeting a splice donor or a splice acceptor. The process needs CRISPR RNA-guided complexes comprising a guide RNA (gRNA), a dCas13 linked to an RNA binding protein (RBP), and a repRNA containing a transgenic gene with RBP-binding hairpins. CRISPR-mediated trans-splicing is achieved by binding the CRISPR-Cas RNP and repRNA complex to a target pre-mRNA. The dCas13-RBP binds to and blocks the cis-splicing acceptor while simultaneously recruiting the splice repRNA, thus enabling efficient and specific trans-splicing to produce a transgenic gene of interest, as shown in FIG. 2.

In some cases, the multi-kilobase replicon RNA (repRNA) comprises a construct, e.g., trans-splicing construct as described herein.

To generate single sex offspring via the enhanced trans-splicing approach, one needs to express the dCas 13-RBP protein, guide RNA, and repRNA early in development from the Z chromosome. The repRNA comprises an open reading frame encoding a transgenic protein (e.g., a toxin), a splice site, an intron with RBP-binding hairpins, and a polyadenylation signal. In some instances, dCas13-RBP binds to the splicing acceptor and recruits the splice repRNA, thus enabling enhanced trans-splicing to produce a transgenic protein (e.g., a toxin).

### Example 3: Design of a Trans-Splicing Construct to Differentiate On-Target and Off-Target Trans-Splicing

RNA trans-splicing is an RNA splicing reaction that occurs between two RNA molecules. Briefly, the spliceosome can splice at the 5′ splice site from one RNA molecule and the branch point, together with the 3′ splice site from a different RNA molecule thereby connecting the two exons from the two different RNA molecules together. A trans-splicing event can be: (i) a 3′ trans-splicing, wherein the 5′ splice site of the target pre-mRNA, e.g., the trans-splicing acceptor gene, is spliced together with the 3′ splice site of a different RNA molecule, e.g., the trans-splicing donor gene or an RNA sequence carrying two or more transgenes and joining the two exons together. Additionally, a trans-splicing event can be: (ii) a 5′ trans-splicing, wherein the 5′ splice site of a different RNA molecule is spliced together with the 3′ splice site of the target pre-mRNA. Alternatively, when trans-splicing events (i) and (ii) occur, exon replacement or insertion of a transgene can be achieved.

In order to create only female offspring, e.g., chickens, that are not genetically modified, the gene being trans-spliced to the endogenous chicken gene must be incompatible with life (such as a toxin) to ensure offspring with the male (ZZ) genotype are not viable. In the event the gene being trans-spliced is a toxin and because trans-splicing is a rare event, it is necessary to select a highly potent toxin. Barnase, which is a nucleic acid degrading toxin, is an example of such a toxin. Since even one molecule of Barnase can kill a cell, the trans-splicing reaction must be specific as a single off-target splicing event can be lethal. In some cases, off-target splicing that occurs in the transcribed reading frame of a transgene, e.g., a toxin, can be lethal for the cell(s) in which it occurs. Alternatively, off-target splicing can result in an out of frame construct, therefore, in some cases, no toxin is produced.

In this example, in order to differentiate between the on-target and off-target trans-splicing process, a trans-splicing construct is designed to only result in the production of an active protein, e.g., a toxin, encoded by the first transgene, when both the 3′ trans-splicing and 5′ trans-splicing events occur. Where both trans-splicing events occur, the result is exon replacement or insertion of the first transgene. The addition of a second transgene located on the 3′ end of the first transgene can be utilized to distinguish between the 3′ trans-splicing, 5′ trans-splicing, or both trans-splicing events. The second transgene can encode, for example, a cognate toxin inhibitor (e.g., wherein the first transgene is Barnase, the second transgene is Barstar). Further, the intron(s) or sequences between these first and second transgenes may not contain a stop codon. This construct can be inserted into an expression cassette.

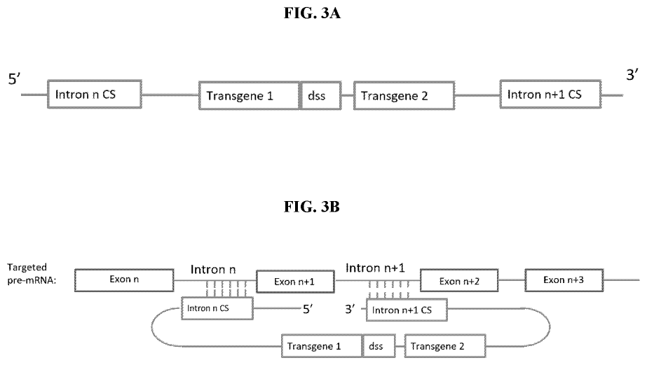

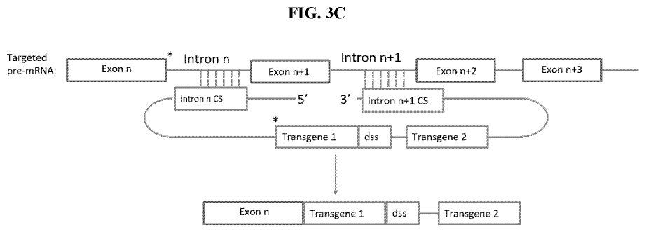

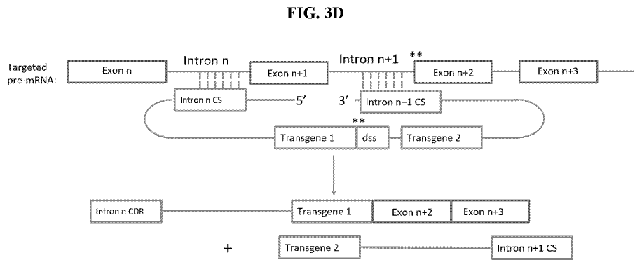

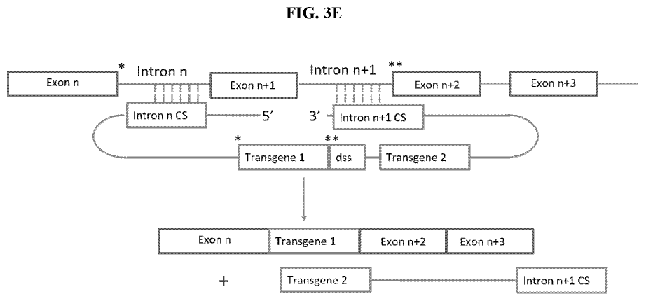

An exemplary diagram of a trans-splicing construct having a first transgene and a second transgene as described herein, which is capable of aiding differentiation of on-target and off-target trans-splicing, is shown in FIG. 3A.

“Intron n CS” is the complementary sequence of the intron located immediately upstream of the exon to be replaced by the trans-splicing event(s).

“ass” is the acceptor splice site (not shown), which is located on the 5′ end of the “Transgene 1.”

“Transgene 1” is a nucleic acid sequence encoding a first protein of interest. “Transgene 1” is designed to (1) not have its own in frame start codon and (2) have the same open reading frame once fused to the targeted mRNA, so that “Transgene 1” can only be translated when trans-splicing event occurs at the acceptor splice site and join to Exon n of the target mRNA Additionally, sequence on the 5′ end of “Transgene 1” is designed to not have Kozak sequence.

“dss” is the donor splice site, which is located on the 3′ end of the “Transgene 1”.

“Transgene 2” is a nucleic acid sequence encoding a second protein of interest, which can be different protein than “Transgene 1”. Importantly, “Transgene 2” shares the same open reading frame as “Transgene 1” such that translation of “Transgene 2” naturally occurs after translation of “Transgene 1”. Off-target trans-splicing events therefore result in both “Transgene 1” and “Transgene 2” being expressed together (see Case 2 below). “Transgene 2” is designed to not include an in-frame start codon (ATG). Also, sequences located between “Transgene 1” and “Transgene 2” do not contain stop codons, so that these two genes are translated on the same polypeptide. Additionally, sequence on the 5′ end of “Transgene 2” is designed to not have Kozak sequence.

“Intron n+1 CS” is the complementary sequence of the intron located immediately downstream of the exon(s) to be replaced.

In some cases, nucleic acid sequence at the 5′ end of the “Transgene 1” can have splicing enhancement sequence, which comprises a branch point (bp) and/or polypyrimidine track (ppt). The splicing enhancement sequence is located on the 5′ end of the acceptor splice site.

To generate single sex offspring, “Transgene 1” is designed to encode a toxin, e.g., a Barnase, and “Transgene 2” is designed to encode a cognate toxin inhibitor, e.g., a Barstar. Expression of the one or more expression cassettes is via a promoter that is active during embryogenesis. As such, the expression of the trans-splicing construct is active only during embryogenesis.

FIG. 3B shows an overview of the transcribed trans-splicing construct (trans-splice donor gene) base pairing to the targeted pre-mRNA (trans-splice acceptor gene). The nucleic acid sequence (complementary sequence) of the trans-splicing construct binds complementary to the target region of the pre-mRNA, e.g., the trans-splice acceptor gene, which has a wildtype sequence. Many splicing events can happen. For example:

### Case 1-No Splicing Occurs.

No proteins encoded by the two or more transgenes are produced from the trans-splicing construct as a result of the start codon, ATG, not being present.

### Case 2-Trans-Splicing Occurs Only at Intron n (3′ Trans-Splicing).

In this case, as shown in FIG. 3C, a fusion mRNA is produced, containing both “Transgene 1” and “Transgene 2”. The encoded protein may be translated. Proteins encoded by “Transgene 1”, e.g., a Barnase, and “transgene 2”, e.g., a Barstar, are produced in a 1 to 1 ratio and on the same polypeptide, therefore, the function of Barnase will be inhibited. Note: * in FIG. 3C indicates splicing and ligating sites.

In this way, the addition of a second transgene (Transgene 2), e.g., a cognate toxin inhibitor, located on the 3′ end of the first transgene (Transgene 1), provides a safety switch preventing cell death due to an unanticipated trans-splicing event at intron n as the toxin is co-translated with its cognate toxin inhibitor. Sequences and/or introns located between “Transgene 1” and “Transgene 2” should contain no stop codons such that the toxin and its respective inhibitor are translated on the same polypeptide. Also, a sequence encoding “Transgene 2” is envisioned to not contain a start codon and can therefore only be expressed if “Transgene 1” expression occurs. Further, because the toxin and the cognate toxin inhibitor are designed to be expressed on the same polypeptide, the probability that the toxin and the cognate toxin inhibitor bind to one another in cis exceeds the probability that the cognate toxin inhibitor binds to a toxin produced by a different splicing reaction (see Case 4 below).

Case 3-Trans-Splicing Occurs Only at Intron n+1 (5′ Trans-Splicing).

In this case, two products of trans-splicing event are shown in FIG. 3D. The first product comprising a “Transgene 1” is not functional, and “Transgene 1” is not translated because of the lack of an in frame start codon and the lack of Kozak sequence. The second product comprises a “Transgene 2”. Because “Transgene 2” lacks an in-frame start codon (ATG), a 5′ cap, and a Kozak sequence; no protein is produced. Note: ** in FIG. 3D indicates splicing and ligating sites.

### Case 4-Both 5′ Trans-Splicing and 3′ Trans-Splicing Occur.

In this case, as shown in FIG. 3E, the exon replacement or insertion of “Transgene 1”, e.g., a toxin such as Barnase, is achieved without having an insertion of “Transgene 2”, e.g., a cognate toxin inhibitor such as Barstar. Therefore, when both 5′ trans-splicing and 3′ trans-splicing occur, the toxic protein encoded by “Transgene 1” is expressed alone without the expression the corresponding inhibitor of Transgene 2. In this case, if the toxic protein encoded by “Transgene 1” is functional as a fusion protein, the resulting cell harboring both trans-splicing events is not viable. This is a desirable outcome for generating single sex offspring as described in FIG. 1.

Case 5-Combinations of Cases 2, 3, and/or 4 Occurring in the Same Cell.

For situations wherein cases 2 and 4 occur in the same cell, expression level of “Transgene 1”, e.g., a toxin such as Barnase, from cases 2 and 4 is higher than an expression level of “Transgene 2”, e.g., a cognate toxin inhibitor such as Barstar, from case 2; therefore, the toxin can still function, and the resulting cell is not viable.

For situations wherein cases 3 and 4 occur in the same cell, the expression of “Transgene 1”, e.g., a toxin, can occur while the protein encoded by “Transgene 2” is not expressed. Therefore, the toxin can still function, and the resulting cell is not viable.

Where both 5′ trans-splicing and 3′ trans-splicing events occur (as described in Case 3), and/or result in the exon replacement or insertion of “Transgene 1”, the toxic protein encoded by “Transgene 1” is expressed, and the resulting cell harboring this event is not viable.

### Case 6-Both 5′ Trans-Splicing and 3′ Trans-Splicing Occur but are Both Off-Target.

This is a rare event, but expression of protein encoded by “Transgene 1” can occur.

Trans-splicing can be an inefficient reaction. Using higher amounts of the trans-splicing construct can increase the chances of off-target splicing events, especially for exon replacement or insertion of a transgene as two trans-splicing reactions are required. However, the construct and system described herein can be utilized in higher amounts to drive trans-splicing reactions without increasing the risk of off-target splicing reactions that would result in cell death. A summary of the trans-splicing events described herein is provided in Table 1.

TABLE 1
Summary of exemplary trans-splicing events.

| | 5′ | 3′ | | |
|---|---|---|---|---|
| Case | Splice | Splice | Outcome | Phenotype |
| 1 | No | No | No protein produced | Alive |
| 2 | Yes | No | Both toxin and | Alive |
| | | | inhibitor are produced | |
| 3 | No | Yes | No protein produced | Alive |
| 4 | Yes | Yes | Only toxin is produced | Dead |
| 5 | n/a | n/a | Toxin is produced in | Dead |
| (combination | | | excess of inhibitor | |
| of cases | | | | |
| 2 and 4) | | | | |

Since the expression of a construct described herein is via a promoter that is only active during embryogenesis, the likelihood of unintended negative impacts in adult animals (e.g., the breeding stock/parent animals) is low. The single sex offspring generated from the constructs, systems, and/or methods described herein is not genetically modified as described in FIG. 1.

In addition to Barnase and Barstar, other toxins and cognate toxin inhibitors as described herein can be used for the construct and/or system described herein.

### Example 4: Use of a Protein Intermediary with a Trans-Splicing Construct to Generate Single Sex Offspring

In order to minimize the off-target trans-splicing events and improve targeting specificity, use of a protein intermediary, e.g., an RNA binding framework such as CRISPR RNA-guided complexes, including those described by Borrajo J et al. “Programmable multi-kilobase RNA editing using CRISPR-mediated trans-splicing.” bioRxiv. 18 Aug. 2023, doi: 10.1101/2023.08.18.553620; Fiflis, David N et al. “Repurposing CRISPR-Cas13 systems for robust mRNA trans-splicing.” Nature communications vol. 15,1 2325. 14 Mar. 2024, doi: 10.1038/s41467-024-46172-4; and US2021/0071178A1, each of which are hereby incorporated by reference of their entities, can be utilized. These protein intermediaries can be used to replace nucleic acid sequence that binds complementary to the target region of the pre-mRNA of the trans-splice acceptor gene containing a wildtype sequence as described in Example 3. These protein intermediaries can improve accuracy (correct splicing events) and/or reduce off-target splicing events.

In this example, the construct and/or system as described in Example 3 can be utilized in conjunction with a protein intermediary, e.g., a CRISPR-Cas RNP and repRNA complex as described in previous examples. One or more protein intermediaries can be located at one or both splice sites.

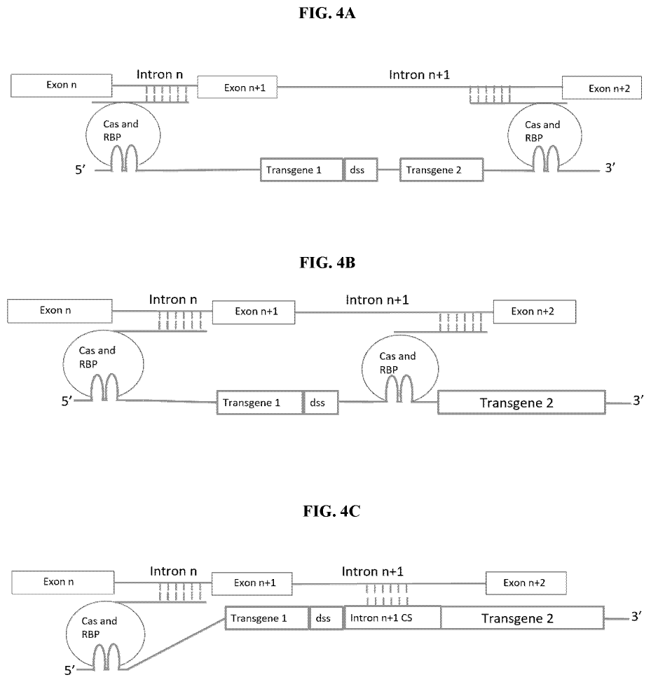

For example, as shown in FIG. 4A, a first intron located on the 5′ end of the acceptor splice site comprises RBP-binding hairpins. The gRNA of this first CRISPR-Cas RNP is directed to the targeted intron n of the targeted mRNA. Additionally, the intron located on the 3′ end of Transgene 2 comprises another RBP-binding hairpin. The gRNA for this second CRISPR-Cas RNP is directed to the targeted intron n+1 of the targeted mRNA.

Alternatively, the protein intermediary can be disposed between “Transgene 1” and “Transgene 2”. As shown in FIG. 4B, a first intron located on the 5′ end of the acceptor splice site comprises RBP-binding hairpins. The gRNA of this first CRISPR-Cas RNP is directed to the targeted intron n of the targeted mRNA. The second intron located on the 3′ end of the dss comprises another RBP-binding hairpin. The gRNA for this second CRISPR-Cas RNP is directed to the targeted intron n+1 of the targeted mRNA. In this case, if trans-splicing event occurs only at the acceptor splice site (ass) located on the 5′ end of the “Transgene 1”, translation of “Transgene 2” might not occur because the RNA secondary structure (hairpin) and/or binding of proteins, e.g., the Cas and RBP, on the transcript can prevent a ribosome from reaching the open reading frame of “Transgene 2”.

If RBP-binding hairpins are utilized to assist trans-splicing at both 5′ and 3′ splice sites of “Transgene 1”, two different RNA binding protein systems are utilized to assist with correct orientation of nucleic acid sequence encoding two or more transgenic proteins, e.g., the “Transgene 1” and “Transgene 2”, to the target pre-mRNA. For example, if same RNA binding protein systems, at both 5′ and 3′ splice sites, are utilized as in diagram shown in FIG. 4A and FIG. 4B, the gRNA of Cas and RBP at the 5′ RBP-binding hairpins can direct the nucleic acid sequence encoding two or more transgenic proteins toward the 3′ splice site of the intron n+1, therefore, the orientation can be incorrect and trans-splicing is unlikely to occur. A similar situation is applied for the second Cas and RBP at the 3′ RBP-binding hairpins. Accordingly, if two different RBP-binding hairpins are utilized, two different protein intermediaries, e.g., a CRISPR-Cas RNP and repRNA complex, are applied here such that the 5′ CRISPR-Cas RNP and repRNA complex for 5′ splice site comprises a unique hairpin and a unique gRNA, which is different than the 3′ CRISPR-Cas RNP and repRNA complex for 3′ splice site. Examples of unique hairpin and binding proteins are provided in Table 2 below.

TABLE 2
Exemplary protein and RNA hairpin

| | RNA Binding | |
|---|---|---|
| Protein | Motif Type | Characteristics |
| MS2 coat | Specific RNA | Recognizes a defined RNA hairpin with a |
| protein | stem-loop | critical uracil or cytosine in the loop. |
| | (hairpin) | |
| Qβ coat | Specific RNA | Similar tertiary structure to MS2 coat |
| protein | stem-loop | protein but having specificity on a |
| | (hairpin) | different RNA sequence. Detailed |
| | | information is described, for example, in |
| | | Lim, F. (1996). J Biol Chem 271(50): |
| | | 31839-45, which is hereby incorporated |
| | | by reference of its entirety. |
| GA coat | Specific RNA | Homologous to MS2 coat protein; distinct |
| protein | stem-loop | RNA loop sequence recognized. Detailed |
| | (hairpin) | information is described, for example, in |
| | | Lim, F. (1996). J Biol Chem 271(50): |
| | | 31839-45, which is hereby incorporated |
| | | by reference of its entirety. |
| PP7 coat | Specific RNA | Different RNA binding mode; used |
| protein | stem-loop | similarly in RNA tagging. Detailed |
| | (hairpin) | information is described, for example, in |
| | | Kaganman, I. (2008). Nature Methods 5, |
| | | 126, which is hereby incorporated by |
| | | reference of its entirety. |

Further, a skilled artisan would be able to customize RNA hairpins and binding proteins, for example, as described in Lim, F. (1994). J Biol Chem 25;269 (12): 9006-10, which is hereby incorporated by reference of its entity.

To improve trans-splicing efficiency and continued translation by a ribosome, as shown in FIG. 4C, a combination of protein intermediaries along with base pairing of complementary sequence can be utilized for trans-splicing. As shown in FIG. 4C, a first intron located on the 5′ end of the acceptor splice site comprises RBP-binding hairpins. The gRNA of this first CRISPR-Cas RNP is directed to the targeted intron n of the targeted mRNA. The sequence located on the 3′ end of the dss is a sequence that is complementary to the sequence of intron n+1. The RBP-binding hairpin is located on the 5′ end of the acceptor splice site to improve on-target trans-splicing, and intron complementary sequence is located on the 3′ end of the donor splice site to allow a ribosome to reach to the open reading frame of “Transgene 2” in case only one trans-splice event occurs at the acceptor splice site.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

1. A non-human vertebrate animal having a modified genotype comprising:
(1) one or more first sequence variants of a gene, wherein the one or more first sequence variants of the gene comprise a modified intron that is not capable of base pairing with a trans-splicing accepting gene; and
(2) one or more expression cassettes comprising: a nucleic acid sequence encoding a replicon RNA (repRNA), comprising a nucleic acid sequence encoding two or more transgenic proteins, two or more splice sites, and one or more introns, wherein the two or more transgenic proteins comprise a first transgenic protein and a second transgenic protein;
wherein a 5′ end of the nucleic acid sequence encoding the first transgenic protein comprises a first splice site and a 3′ end of the nucleic acid sequence encoding the first transgenic protein comprises a second splice site;
wherein at least one of the one or more introns does not comprise a stop codon; and
wherein at least one of the one or more introns comprises a sequence that is complementary to one or more second sequence variants of the gene.

2. The non-human vertebrate animal of claim 1, wherein translation of the first transgenic protein, but not the second transgenic protein, is initiated when RNA trans-splicing occurs at both the first splice site and the second splice site.

3. The non-human vertebrate animal of claim 1, wherein translation of each of the first transgenic protein and the second transgenic protein is initiated when RNA trans-splicing occurs only at the first splice site.

4. The non-human vertebrate animal of claim 1, wherein the first transgenic protein is located on a 5′ end of the second transgenic protein.

5. The non-human vertebrate animal of claim 1, wherein the first splice site is an acceptor splice site, and the second splice site is a donor splice site.

6. (canceled)

7. The non-human vertebrate animal of claim 1, wherein the first transgenic protein is different than the second transgenic protein.

8. The non-human vertebrate animal of claim 1, wherein the first transgenic protein is a reporter gene or a toxin.

9. (canceled)

10. The non-human vertebrate animal of claim 8, wherein the toxin comprises a nuclease or a protease.

11. (canceled)

12. (canceled)

13. The non-human vertebrate animal of claim 1, wherein the second transgenic protein is a cognate toxin inhibitor.

14. (canceled)

15. (canceled)

16. The non-human vertebrate animal of claim 1, wherein:
(i) the first transgenic protein is a Barnase or a variant thereof, and the second transgenic protein is a Barstar or a variant thereof;
(ii) the first transgenic protein is an RNase, and the second transgenic protein is an RNase inhibitor;
(iii) the first transgenic protein is a restriction endonuclease, and the second transgenic protein is a restriction endonuclease inhibitor;
(iv) the first transgenic protein is a diphtheria, and the second transgenic protein is a diphtheria antitoxin;
(v) the first transgenic protein is a ricin, and the second transgenic protein is a ricin inhibitor;
(vi) the first transgenic protein is an abrin, and the second transgenic protein is an abrin inhibitor;
(vii) the first transgenic protein is a pokeweed antiviral protein, and the second transgenic protein is a pokeweed antiviral protein inhibitor;
(viii) the first transgenic protein is a caspase, and the second transgenic protein is a caspase inhibitor;
(ix) the first transgenic protein is a trypsin, and the second transgenic protein is a trypsin inhibitor;
(x) the first transgenic protein is a chymotrypsin, and the second transgenic protein is a chymotrypsin inhibitor; or
(xi) the first transgenic protein is a papain, and the second transgenic protein is a papain inhibitor.

17. The non-human vertebrate animal of claim 1, wherein the one or more expression cassettes further comprises a second nucleic acid sequence encoding a Cas polypeptide linked to an RNA Binding Protein (RBP) and a third nucleic acid sequence encoding a guide RNA (gRNA) capable of directing one or more CRISPR RNA-guided complexes encoded by the one or more expression cassettes to one or more second sequence variants of the gene.

18. The non-human vertebrate animal of claim 17, wherein the one or more introns comprises:
a. a first intron located on the 5′ end of the first transgenic protein, and wherein the first intron comprises a first RBP-binding hairpin; and
b. a second intron located on the 3′ end of the first transgenic protein, and wherein the second intron comprises a second RBP-binding hairpin.

19. (canceled)

20. The non-human vertebrate animal of claim 17, wherein a second RBP-binding hairpin is:
a. on a nucleic acid sequence located on a 3′ end of the second transgenic protein; or
b. on a nucleic acid sequence located on the 5′ end of the second transgenic protein.

21. (canceled)

22. The non-human vertebrate animal of claim 17,
wherein the one or more introns comprises a first intron located on the 3′ end of the first transgenic protein, and wherein the first intron comprises a first RBP-binding hairpin.

23-28. (canceled)

29. The non-human vertebrate animal of claim 1, wherein the non-human vertebrate animal is selected from the group consisting of cow, mouse, rat, rabbit, guinea pig, chicken, fish, bird, reptile, camelid, bovine, chimpanzee, sheep, goat, and non-human primate.

30. The non-human vertebrate animal of claim 1, wherein the one or more second sequence variants of the gene;
a. do not have sequence identity to the one or more first sequence variant of the gene; or
b. have sequence identity to a wildtype sequence of the gene.

31. (canceled)

32. (canceled)

33. (canceled)

34. The non-human vertebrate animal of claim 1, wherein the Cas polypeptide is a Cas endonuclease.

35-47. (canceled)

48. The non-human vertebrate animal of claim 17, wherein the one or more CRISPR RNA-guided complexes comprise the guide RNA, the Cas polypeptide, the repRNA, or a combination thereof.

49-104. (canceled)

105. A method of producing a single sex population of non-human vertebrate animals, the method comprising:
(1) crossing the first non-human vertebrate animal having a modified genotype comprising:
(i) one or more first sequence variants of a gene, wherein the one or more first sequence variants of the gene comprise a modified intron that is not capable of base pairing with a trans-splicing accepting gene; and
(ii) one or more expression cassettes comprising: a nucleic acid sequence encoding a replicon RNA (repRNA), comprising a nucleic acid sequence encoding two or more transgenic proteins, two or more splice sites, and one or more introns,
wherein the two or more transgenic proteins comprise a first transgenic protein and a second transgenic protein;
wherein a 5′ end of the nucleic acid sequence encoding the first transgenic protein comprises a first splice site and a 3′ end of the nucleic acid sequence encoding the first transgenic protein comprises a second splice site;
wherein at least one of the one or more introns does not comprise a stop codon; and
wherein at least one of the one or more introns comprises a sequence that is complementary to one or more second sequence variants of the gene;
(2) with a second non-human vertebrate animal comprising the one or more second variants of the gene;
wherein a resulting progeny having a genotype comprising the one or more second sequence variants of the gene and the one or more expression cassettes is not viable; thereby producing the single sex population of non-human vertebrate animals, wherein each of the non-human vertebrate animals of the single sex population of non-human vertebrate animals is not genetically engineered.

106. (canceled)

107. (canceled)

108. (canceled)

109. A plurality of non-human vertebrate animals comprising:
(1) a first non-human vertebrate animal having a genotype comprising
(i) one or more first sequence variants of a gene, wherein the one or more first sequence variants of the gene comprise a modified intron that is not capable of base pairing with a trans-splicing accepting gene; and
(ii) one or more expression cassettes comprising:
a first nucleic acid sequence encoding a Cas polypeptide linked to an RNA Binding Protein (RBP);
a second nucleic acid sequence encoding a replicon RNA (repRNA),
comprising a nucleic acid sequence encoding two or more transgenic proteins, two or more splice sites, and one or more introns,
wherein the two or more transgenic proteins comprise a first transgenic protein and a second transgenic protein;
wherein a 5′ end of the nucleic acid sequence encoding the first transgenic protein comprise a first splice site and a 3′ end of the nucleic acid sequence encoding the first transgenic protein comprises a second splice site; and
wherein at least one of the one or more introns (a) is connected to an RBP-binding hairpin, and/or (b) does not comprise a stop codon; and
a third nucleic acid sequence encoding a guide RNA (gRNA) capable of directing one or more CRISPR RNA-guided complexes encoded by the one or more expression cassettes to one or more second sequence variants of the gene; and
(2) a second non-human vertebrate animal comprising the one or more second variants of the gene.

110. (canceled)
